(19) United States
(12) Patent Application Publication
(10) Pub. No.: US 2019/0055564 A1
(43) Pub. Date: Feb. 21, 2019

(54) ANTISENSE OLIGONUCLEOTIDES FOR MODULATING HTRA1 EXPRESSION

(71) Applicants: F. Hoffmann-La Roche AG, Basel (CH); Roche Innovation Center Copenhagen A/S, Horsholm (DK)

(72) Inventors: Rubén Alvarez Sánchez, Basel (CH); Roberto Iacone, Basel (CH); Peter Hagedorn, Hørsholm (DK); Susanne Kammler, Hørsholm (DK); Søren Ottosen, Hørsholm (DK); Sindri Traustason, Hørsholm (DK); Heidi Rye Hudlebusch, Hørsholm (DK); Lykke Pedersen, Hørsholm (DK); Marco Berrera, Basel (CH); Andreas Dieckmann, Basel (CH)

(21) Appl. No.: 15/991,326

(22) Filed: May 29, 2018

(30) Foreign Application Priority Data
Jun. 1, 2017 (EP) 17173964.2
Dec. 21, 2017 (EP) 17209407.0
Dec. 21, 2017 (EP) 17209535.8

(51) Int. Cl. C12N 15/113; A61P 27/02

(52) U.S. Cl. CPC C12N 15/1137; A61P 27/02; C12N 2310/351; C12N 2310/315; C12N 2310/3231; C12N 2310/11

(57) ABSTRACT

The present invention relates to antisense oligonucleotides (oligomers) that are complementary to HTRA1, leading to modulation of the expression of HTRA1. Modulation of HTRA1 expression is beneficial for a range of medical disorders, such as macular degeneration, e.g. age-related macular degeneration.

### RELATED APPLICATIONS

This application claims priority to EP 17173964.2, filed Jun. 1, 2017, 17209407.0, filed Dec. 21, 2017 and 17209535.8, filed Dec. 21, 2017. The contents of which are hereby incorporated by reference in their entireties.

### FIELD OF INVENTION

The present invention relates to antisense oligonucleotides (oligomers) that are complementary to HTRA1, leading to modulation of the expression of HTRA1. Modulation of HTRA1 expression is beneficial for a range of medical disorders, such as macular degeneration, e.g. age-related macular degeneration.

### BACKGROUND

The human high temperature requirement A (HTRA) family of serine proteases are ubiquitously expressed PDZ-proteases that are involved in maintaining protein homeostasis in extracellular compartments by combining the dual functions of a protease and a chaperone. HTRA proteases are implicated in organization of the extracellular matrix, cell proliferation and ageing. Modulation of HTRA activity is connected with severe diseases, including Duchenne muscular dystrophy (Bakay et al. 2002, Neuromuscul. Disord. 12: 125-141), arthritis, such as osteoarthritis (Grau et al. 2006, JBC 281: 6124-6129), cancer, familial ischemic cerebral small-vessel disease and age-related macular degeneration, as well as Parkinson's disease and Alzheimer's disease. The human HTRA1 contains an insulin-like growth factor (IGF) binding domain. It has been proposed to regulate IGF availability and cell growth (Zumbrunn and Trueb, 1996, FEES Letters 398:189-192) and to exhibit tumor suppressor properties. HTRA1 expression is down-regulated in metastatic melanoma, and may thus indicate the degree of melanoma progression. Overexpression of HTRA1 in a metastatic melanoma cell line reduced proliferation and invasion in vitro, and reduced tumor growth in a xenograft mouse model (Baldi et al., 2002, Oncogene 21:6684-6688). HTRA1 expression is also down-regulated in ovarian cancer. In ovarian cancer cell lines, HTRA1 overexpression induces cell death, while antisense HTRA1 expression promoted anchorage-independent growth (Chien et al., 2004, Oncogene 23:1636-1644).

In addition to its effect on the IGF pathway, HTRA1 also inhibits signaling by the TGFβ family of growth factors (Oka et al., 2004, Development 131:1041-1053). HTRA1 can cleave amyloid precursor protein (APP), and HTRA1 inhibitors cause the accumulation of Aβ peptide in cultured cells. Thus, HTRA1 is also implicated in Alzheimer's disease (Grau et al., 2005, Proc. Nat. Acad. Sci. USA. 102:6021-6026).

Furthermore, HTRA1 upregulation has been observed and seems to be associated to Duchenne muscular dystrophy (Bakay et al. 2002, Neuromuscul. Disord. 12: 125-141) and osteoarthritis (Grau et al. 2006, JBC 281: 6124-6129) and AMD (Fritsche, et al. Nat Gen 2013 45(4):433-9.)

A single nucleotide polymorphism (SNP) in the HTRA1 promoter region (rs11200638) is associated with a 10 fold increased the risk of developing age-related macular degeneration (AMD). Moreover the HTRA1 SNPs are in linkage disequilibrium with the ARMS2 SNP (rs10490924) associated with increased risk of developing age-related macular degeneration (AMD). The risk allele is associated with 2-3 fold increased HTRA1 mRNA and protein expression, and HTRA1 is present in drusen in patients with AMD (Dewan et al., 2006, Science 314:989-992; Yang et al., 2006, Science 314:992-993). Over-expression of HtrA1 Induces AMD-like phenotype in mice. The hHTRA transgenic mouse (Veierkottn, PlosOne 2011) reveals degradation of the elastic lamina of Bruch's membrane, determines choroidal vascular abnormalities (Jones, PNAS 2011) and increases the Polypoidal choroidal vasculopathy (PCV) lesions (Kumar, IOVS 2014). Additionally it has been reported that Bruch's membrane damage in hHTRA1 Tg mice, which determines upon exposure to cigarette smoke 3 fold increases CNV (Nakayama, IOVS 2014)

Age-related macular degeneration (AMD) is the leading cause of irreversible loss of vision in people over the age of 65. With onset of AMD there is gradual loss of the light sensitive photoreceptor cells in the back of the eye, the underlying pigment epithelial cells that support them metabolically, and the sharp central vision they provide. Age is the major risk factor for the onset of AMD: the likelihood of developing AMD triples after age 55. Smoking, light iris color, sex (women are at greater risk), obesity, and repeated exposure to UV radiation also increase the risk of AMD. AMD progression can be defined in three stages: 1) early, 2) intermediate, and 3) advanced AMD. There are two forms of advanced AMD: dry AMD (also called geographic atrophy, GA) and wet AMD (also known as exudative AMD). Dry AMD is characterized by loss of photoreceptors and retinal pigment epithelium cells, leading to visual loss. Wet AMD, is associated with pathologic choroidal (also referred to as subretinal) neovascularization. Leakage from abnormal blood vessels forming in this process damages the macula and impairs vision, eventually leading to blindness. In some cases, patients can present pathologies associated with both types of advanced AMD. Treatment strategies for wet AMD require frequent injections into the eye and are focused mainly on delaying the disease progression. Currently no treatment is available for dry AMD. There is therefore an unmet medical need in the provision of effective drugs to treat macular degenerative conditions such as wet and dry AMD. WO 2008/013893 claims a composition for treating a subject suffering from age related macular degeneration comprising a nucleic acid molecules comprising an antisense sequence that hybridizes to HTRA1 gene or mRNA: No antisense molecules are disclosed. WO2009/006460 provides siRNAs targeting HTRA1 and their use in treating AMD.

### OBJECTIVE OF THE INVENTION

The present invention provides antisense oligonucleotides which modulate HTRA1 in vivo or in vitro. The invention identified cryptic target sequence motifs present in the human HTRA1 mRNA (including pre-mRNA) which may be targeted by antisense oligonucleotides to give effective HTRA1 inhibition. The invention also provides effective antisense oligonucleotide sequences and compounds which are capable of inhibiting HTRA1, and their use in treatment of diseases or disorders where HTRA1 is indicated.

### SUMMARY OF INVENTION

The present invention relates to oligonucleotides targeting a mammalian HTRA1 nucleic acid, i.e. are capable of inhibiting the expression of HTRA1 and to treat or prevent diseases related to the functioning of the HTRA1. The oligonucleotides targeting HTRA1 are antisense oligonucleotides, i.e. are complementary to their HTRA1 nucleic acid target.

The oligonucleotide of the invention may be in the form of a pharmaceutically acceptable salt, such as a sodium salt or a potassium salt.

Accordingly, the invention provides antisense oligonucleotides which comprise a contiguous nucleotide sequence of 10-30 nucleotides in length with at least 90% complementarity, such as fully complementary to a mammalian HTRA1 nucleic acid, such as SEQ ID NO 1, SEQ ID NO 2, SEQ ID NO 3 or SEQ ID NO 4.

In a further aspect, the invention provides pharmaceutical compositions comprising the oligonucleotides of the invention and pharmaceutically acceptable diluents, carriers, salts and/or adjuvants.

The invention provides LNA antisense oligonucleotides, such as LNA gapmer oligonucleotides, which comprise a contiguous nucleotide sequence of 10-30 nucleotides in length with at least 90% complementarity, such as fully complementary to a HTRA1 nucleic acid, such as a sequence selected from the group consisting of SEQ ID NO 1, SEQ ID NO 2, SEQ ID NO 3 or SEQ ID NO 4.

The invention provides for an antisense oligonucleotide comprising a contiguous nucleotide region of at 10-30, such as 12-22, nucleotides, wherein the contiguous nucleotide region is at least 90% such as 100% complementary to SEQ ID NO 113.

The invention provides for an antisense oligonucleotide of 10-30 nucleotides in length, wherein said antisense oligonucleotide comprises a contiguous nucleotide region of 10-30, such as 12-22, nucleotides which are at least 90% such as 100% complementarity to SEQ ID NO 113:

5′
GACAGTCAGCATTTGTCTCCTCCTTTAACTGAGTCATCATCTTAGTCCAA
CTAATGCAGTCGATACAATGCGTAGATAGAAGAAGCCCCACGGGAGCCAG
GATGGGACTGGTCGTGTTTGTGCTTTTCTCCAAGTCAGCACCCAAAGGTC
AATGCACAGAGACCCCGGGTGGGTGAGCGCTGGCTTCTCAAACGGCCGAA
GTTGCCTCTTTTAGGAATCTCTTTGGAATTGGGAGCACGATGACTCTGAG
TTTGAGCTATTAAAGTACTTCTTAC 3′.

The reverse complement of SEQ ID NO 113 is SEQ ID NO 119:

GTAAGAAGTACTTTAATAGCTCAAACTCAGAGTCATCGTGCTCCCAATTC
CAAAGAGATTCCTAAAAGAGGCAACTTCGGCCGTTTGAGAAGCCAGCGCT
CACCCACCCGGGGTCTCTGTGCATTGACCTTTGGGTGCTGACTTGGAGAA
AAGCACAAACACGACCAGTCCCATCCTGGCTCCCGTGGGGCTTCTTCTAT
CTACGCATTGTATCGACTGCATTAGTTGGACTAAGATGATGACTCAGTTA
AAGGAGGAGACAAATGCTGACTGTC.

The invention provides for an antisense oligonucleotide comprising a contiguous nucleotide region of at 10-30, such as 12-22, nucleotides, wherein the contiguous nucleotide region is at least 90% such as 100% complementary to SEQ ID NO 114.

The invention provides for an antisense oligonucleotide of 10-30 nucleotides in length, wherein said antisense oligonucleotide comprises a contiguous nucleotide region of 10-30, such as 12-22 nucleotides which are at least 90% such as 100% complementarity to SEQ ID NO 114:

5′
GACAGTCAGCATTTGTCTCCTCCTTTAACTGAGTCATCATCTTAGTCCAA
CTAATGCAGTCGATACAATGCGTAGATAGAAGAAGCCCCACGGGAGCCAG
GATGGGACTGGTCGTGTTTGTGCTTTTCTCCAAGTCAGCACCCAAAGGTC
AATGCACAGAGACCCCGGGTGGGTGAGCGCTGGCTTCTCAAACGGCCGAA
GTTGCCTCTTTTAGGAATCTCTTTGGAATTGGGAGCACGATGACTCTGAG
TTTGAGCTATTAAAGTACTTCTTACACATTGC 3′.

The reverse complement of SEQ ID NO 114 is SEQ ID NO 120:

GCAATGTGTAAGAAGTACTTTAATAGCTCAAACTCAGAGTCATCGTGCTC
CCAATTCCAAAGAGATTCCTAAAAGAGGCAACTTCGGCCGTTTGAGAAGC
CAGCGCTCACCCACCCGGGGTCTCTGTGCATTGACCTTTGGGTGCTGACT
TGGAGAAAAGCACAAACACGACCAGTCCCATCCTGGCTCCCGTGGGGCTT
CTTCTATCTACGCATTGTATCGACTGCATTAGTTGGACTAAGATGATGAC
TCAGTTAAAGGAGGAGACAAATGCTGACTGTC.

The invention provides for an antisense oligonucleotide comprising a contiguous nucleotide region of at 10-30, such as 12-22, nucleotides, wherein the contiguous nucleotide region is at least 90% such as 100% complementary to SEQ ID NO 115.

The invention provides for an antisense oligonucleotide of 10-30 nucleotides in length, wherein said antisense oligonucleotide comprises a contiguous nucleotide region of 1030, such as 12-22 nucleotides which are at least 90% such as 100% complementarity to SEQ ID NO 115:

5′ GACAGTCAGCATTTGTCTCCTCCTTTAACTGAGTCATCATCTTAGTC
CAACTAATGCAGTCGATACAATGCGTAGATAGAAGAAGCCCCACGGGAGC
CAGGATGGGACTGGTCGTGTTTGTGCTTTTCTCCAAGTCAGCACCCAAAG
GTCAATGCACAGAGACCCCGGGTGGGTGAGCGCTGGCTTCTCAAACGGCC
GAAGTTGCCTCTTTTAGGAATCTCTTTGGAATTGGGAGCACGATGACTCT
GAGTTTGAGCTATTAAAGT 3′.

The reverse complement of SEQ ID NO 115 is SEQ ID NO 121:

ACTTTAATAGCTCAAACTCAGAGTCATCGTGCTCCCAATTCCAAAGAGAT
TCCTAAAAGAGGCAACTTCGGCCGTTTGAGAAGCCAGCGCTCACCCACCC
GGGGTCTCTGTGCATTGACCTTTGGGTGCTGACTTGGAGAAAAGCACAAA
CACGACCAGTCCCATCCTGGCTCCCGTGGGGCTTCTTCTATCTACGCATT
GTATCGACTGCATTAGTTGGACTAAGATGATGACTCAGTTAAAGGAGGAG
ACAAATGCTGACTGTC.

The invention provides for an antisense oligonucleotide comprising a contiguous nucleotide region of at 10-30, such as 12-22, nucleotides, wherein the contiguous nucleotide region is at least 90% such as 100% complementary to SEQ ID NO 116.

The invention provides for an antisense oligonucleotide of 10-30 nucleotides in length, wherein said antisense oligonucleotide comprises a contiguous nucleotide region of 10-30, such as 12-22 nucleotides which are at least 90% such as 100% complementarity to SEQ ID NO 116:

5′ CAACTAATGCAGTCGATACAATGCGTAGATAGAAGAAGCCCCACGGG
AGCCAGGATGGGACTGGTCGTGTTTGTGCTTTTCTCCAAGTCAGCACCCA
AAGGTCAATGCACAGAGACCCCGGGTGGGTGAGCGCTGGCTTCTCAAACG
GCCGAAGTTGCCTCTTTTAGGAATCTCTTTGGAATTGGGAGCACGATGAC
TCTGAGTTTGAGCTATTAAAGTACTTCTTACACATTGC 3′.

The reverse complement of SEQ ID NO 116 is SEQ ID NO 122:

GCAATGTGTAAGAAGTACTTTAATAGCTCAAACTCAGAGTCATCGTGCTC
CCAATTCCAAAGAGATTCCTAAAAGAGGCAACTTCGGCCGTTTGAGAAGC
CAGCGCTCACCCACCCGGGGTCTCTGTGCATTGACCTTTGGGTGCTGACT
TGGAGAAAAGCACAAACACGACCAGTCCCATCCTGGCTCCCGTGGGGCTT
CTTCTATCTACGCATTGTATCGACTGCATTAGTTG.

The invention provides for an antisense oligonucleotide comprising a contiguous nucleotide region of at 10-30, such as 12-22, nucleotides, wherein the contiguous nucleotide region is at least 90% such as 100% complementary to SEQ ID NO 117.

The invention provides for an antisense oligonucleotide of 10-30 nucleotides in length, wherein said antisense oligonucleotide comprises a contiguous nucleotide region of 10-30, such as 12-22 nucleotides which are at least 90% such as 100% complementarity to SEQ ID NO 117:

5′ CAACTAATGCAGTCGATACAATGCGTAGATAGAAGAAGCCCCACGGG
AGCCAGGATGGGACTGGTCGTGTTTGTGCTTTTCTCCAAGTCAGCACCCA
AAGGTCAATGCACAGAGACCCCGGGTGGGTGAGCGCTGGCTTCTCAAACG
GCCGAAGTTGCCTCTTTTAGGAATCTCTTTGGAATTGGGAGCACGATGAC
TCTGAGTTTGAGCTATTAAAGTTACTTCTTAC 3′.

The reverse complement of SEQ ID NO 117 is SEQ ID NO 123:

GTAAGAAGTAACTTTAATAGCTCAAACTCAGAGTCATCGTGCTCCCAATT
CCAAAGAGATTCCTAAAAGAGGCAACTTCGGCCGTTTGAGAAGCCAGCGC
TCACCCACCCGGGGTCTCTGTGCATTGACCTTTGGGTGCTGACTTGGAGA
AAAGCACAAACACGACCAGTCCCATCCTGGCTCCCGTGGGGCTTCTTCTA
TCTACGCATTGTATCGACTGCATTAGTTG.

In some embodiments the antisense oligonucleotide of the invention is not of sequence 5′ gcaatgtgtaagaagt 3′ (SEQ ID NO 112). In some embodiments the antisense oligonucleotide of the invention does not comprise or consist of sequence 5′ gcaatgtgtaagaagt 3′. In some embodiments the antisense oligonucleotide of the invention does not comprise or consist of 10 or more contiguous nucleotides present in sequence 5′ gcaatgtgtaagaagt 3′. In some embodiments the oligonucleotide of the invention is other than 5′ GCAatgtgtaagaAGT 3′, wherein Capital letters represent LNA nucleosides (beta-D-oxy LNA nucleosides were used), all LNA cytosines are 5-methyl cytosine, lower case letters represent DNA nucleosides, DNA cytosines preceded with a superscript m represents a 5-methyl C-DNA nucleoside. All internucleoside linkages are phosphorothioate internucleoside linkages.

The invention provides an antisense oligonucleotide which comprises a contiguous nucleotide region of at least 10 contiguous nucleotides present in any one of SEQ ID NOs 5-111. The invention provides an antisense oligonucleotide which comprises a contiguous nucleotide region of at least 12 contiguous nucleotides present in any one of SEQ ID NOs 5-111. The invention provides an antisense oligonucleotide which comprises a contiguous nucleotide region of at least 14 contiguous nucleotides present in any one of SEQ ID NOs 5-111. The invention provides an antisense oligonucleotide which comprises a contiguous nucleotide region of at least 15 or 16 contiguous nucleotides present in any one of SEQ ID NOs 5-111. The invention provides an antisense oligonucleotide, wherein the contiguous nucleotide sequence of the oligonucleotide comprises or consists of a nucleobase sequence selected from the group consisting of any one of SEQ ID NOs 5-111.

The invention provides an antisense oligonucleotide which comprises a contiguous nucleotide region of at least 10, or at least 12, at least 13, or at least 14 or at least 15 or at least 16 contiguous nucleotides present SEQ ID NO 118: 5′ CTTCTTCTATCTACGCATTG 3′. The reverse complement of SEQ ID NO 118 is SEQ ID NO 231: CAATGCGTAGATAGAAGAAG.

The invention provides an antisense oligonucleotide which comprises a contiguous nucleotide region of at least 10, or at least 12, at least 13, or at least 14 or at least 15 or at least 16 contiguous nucleotides complementary to SEQ ID NO 231.

The invention provides an antisense oligonucleotide which comprises a contiguous nucleotide region of at least 10, or at least 12, or at least 13, or at least 14 or at least 15 or 16 contiguous nucleotides present SEQ ID NO 67.

The invention provides an antisense oligonucleotide which comprises a contiguous nucleotide region of at least 10, or at least 12, or at least 13, or at least 14 or at least 15 or 16 contiguous nucleotides present SEQ ID NO 86.

The invention provides an antisense oligonucleotide which comprises a contiguous nucleotide region of at least 10, or at least 12, or at least 13, or at least 14 or at least 15 or at least 16 or at least 17 or 18 contiguous nucleotides present SEQ ID NO 73.

The invention provides an antisense oligonucleotide which comprises a contiguous nucleotide region of at least 10, or at least 12, or at least 13, or at least 14 or at least 15 or 16 contiguous nucleotides complementary to SEQ ID NO 186.

The invention provides an antisense oligonucleotide which comprises a contiguous nucleotide region of at least 10, or at least 12, or at least 13, or at least 14 or at least 15 or 16 contiguous nucleotides complementary to SEQ ID NO 205.

The invention provides an antisense oligonucleotide which comprises a contiguous nucleotide region of at least 10, or at least 12, or at least 13, or at least 14 or at least 15 or at least 16 or at least 17 or 18 contiguous nucleotides complementary to SEQ ID NO 192.

The invention provides for an oligonucleotide comprising or consisting of an oligonucleotide selected from the group consisting of:

(SEQ ID NO 67.1)

TₛTₛ^{m}Cₛtₛaₛtₛcₛtₛaₛ^{m}cₛgₛcₛaₛTₛTₛG,

(SEQ ID NO 73.1)

^{m}CₛTₛTₛ^{m}Cₛtₛtₛcₛtₛaₛtₛcₛtₛaₛ^{m}cₛgₛcₛAₛT,
and

(SEQ ID NO 86.1)

TₛAₛ^{m}CₛTₛtₛtₛaₛaₛtₛaₛgₛcₛTₛ^{m}CₛAₛA;

wherein capital letters represent beta-D-oxy LNA nucleosides, lower case letters are DNA nucleosides, subscript s represents a phosphorothioate internucleoside linkage, and ^{m}C represent 5 methyl cytosine beta-D-oxy LNA nucleosides, and ^{m}c represents 5 methyl cytosine DNA nucleosides.

The invention provides for an oligonucleotide of formula:

(SEQ ID NO 67.1)

TₛTₛ^{m}Cₛtₛaₛtₛcₛtₛaₛ^{m}cₛgₛcₛaₛTₛTₛG,

wherein capital letters represent beta-D-oxy LNA nucleosides, lower case letters are DNA nucleosides, subscript s represents a phosphorothioate internucleoside linkage, and ^{m}C represent 5 methyl cytosine beta-D-oxy LNA nucleosides, and ^{m}c represents 5 methyl cytosine DNA nucleosides.

The invention provides for an oligonucleotide of formula:

(SEQ ID NO 73.1)

^{m}CₛTₛTₛ^{m}Cₛtₛtₛcₛtₛaₛtₛcₛtₛaₛ^{m}cₛgₛcₛAₛT

wherein capital letters represent beta-D-oxy LNA nucleosides, lower case letters are DNA nucleosides, subscript s represents a phosphorothioate internucleoside linkage, and ^{m}C represent 5 methyl cytosine beta-D-oxy LNA nucleosides, and ^{m}c represents 5 methyl cytosine DNA nucleosides.

The invention provides for an oligonucleotide of formula:

(SEQ ID NO 86.1)

TₛAₛ^{m}CₛTₛtₛtₛaₛaₛtₛaₛgₛcₛTₛ^{m}CₛAₛA

wherein capital letters represent beta-D-oxy LNA nucleosides, lower case letters are DNA nucleosides, subscript s represents a phosphorothioate internucleoside linkage, and ^{m}C represent 5 methyl cytosine beta-D-oxy LNA nucleosides, and ^{m}c represents 5 methyl cytosine DNA nucleosides.

The invention provides for the oligonucleotides provided in the examples.

The invention provides for a conjugate comprising the oligonucleotide according to the invention, and at least one conjugate moiety covalently attached to said oligonucleotide.

The invention provides for a pharmaceutically acceptable salt of the oligonucleotide or conjugate of the invention.

In a further aspect, the invention provides methods for in vivo or in vitro method for modulation of HTRA1 expression in a cell which is expressing HTRA1, by administering an oligonucleotide, conjugate or composition of the invention in an effective amount to said cell.

In a further aspect the invention provides methods for treating or preventing a disease, disorder or dysfunction associated with in vivo activity of HTRA1 comprising administering a therapeutically or prophylactically effective amount of the oligonucleotide of the invention, or conjugate thereof, to a subject suffering from or susceptible to the disease, disorder or dysfunction.

In a further aspect the oligonucleotide or composition of the invention is used for the treatment or prevention of macular degeneration, and other disorders where HTRA1 is implicated.

The invention provides for the oligonucleotide or conjugate of the invention, for use in the treatment of a disease or disorder selected from the list comprising of Duchenne muscular dystrophy, arthritis, such as osteoarthritis, familial ischemic cerebral small-vessel disease, Alzhiemer's disease and Parkinson's disease.

The invention provides for the oligonucleotide or conjugate of the invention, for use in the treatment of macular degeneration, such as wet or dry age related macular degeneration (e.g. wAMD, dAMD, geographic atrophy, early AMD, intermediate AMD) or diabetic retinopathy.

The invention provides for the use of the oligonucleotide, conjugate or composition of the invention, for the manufacture of a medicament for the treatment of macular degeneration, such as wet or dry age related macular degeneration (e.g. wAMD, dAMD, geographic atrophy, intermediate dAMD) or diabetic retinopathy.

The invention provides for the use of the oligonucleotide, conjugate or composition of the invention, for the manufacture of a medicament for the treatment of a disease or disorder selected from the group consisting of Duchenne muscular dystrophy, arthritis, such as osteoarthritis, familial ischemic cerebral small-vessel disease, Alzhiemer's disease and Parkinson's disease.

The invention provides for a method of treatment of a subject suffering from a disease or disorder selected from the group consisting of Duchenne muscular dystrophy, arthritis, such as osteoarthritis, familial ischemic cerebral small-vessel disease, Alzhiemer's disease and Parkinson's disease, said method comprising the step of administering an effective amount of the oligonucleotide, conjugate or composition of the invention to the subject.

The invention provides for a method of treatment of a subject suffering from an ocular disease, such as macular degeneration, such as wet or dry age related macular degeneration (e.g. wAMD, dAMD, geographic atrophy, intermediate dAMD) or diabetic retinopathy, said method comprising the step of administering an effective amount of the oligonucleotide, conjugate or composition of the invention to the subject.

The invention provides for a method of treatment of a subject suffering from an ocular disease, such as macular degeneration, such as wet or dry age related macular degeneration (e.g. wAMD, dAMD, geographic atrophy, intermediate AMD) or diabetic retinopathy, said method comprising administering at least two dosages of the oligonucleotide of the invention, or pharmaceutically acceptable salt thereof, in an intraocular injection in a dosage of from about 10 μg-200 μg, wherein the dosage interval between administration consecutive is at least 4 weeks (i.e. a dosage interval is 4 weeks), or at least monthly (i.e. a dosage interval is 1 month).

### BRIEF DESCRIPTION OF FIGURES

FIG. 1. A library of n=231 HTRA1 LNA oligonucleotides were screened in U251 cell lines at 5 μM. The residual HTRA1 mRNA expression level was measured by qPCR and is shown as % of control (PBS-treated cells). n=10 oligos located between position 53113-53384 were relatively active.

FIG. 2. A library of n=210 HTRA1 LNA oligonucleotides were screened in U251 cell lines at 5 μM. The residual HTRA1 mRNA expression level was measured by qPCR and is shown as % of control (PBS-treated cells). n=33 oligos located between position 53113-53384 were relatively active.

FIG. 3. A library of n=305 HTRA1 LNA oligonucleotides were screened in U251 and ARPE19 cell lines at 5 and 25 μM, respectively. The residual HTRA1 mRNA expression level was measured by qPCR and is shown as % of control (PBS-treated cells). n=95 oligos located between position 53113-53384 were relatively active in comparison to the rest.

FIG. 4. Dose response of HTRA1 mRNA level upon treatment of human primary RPE cells with LNA oligonucleotides, 10 days of treatment. Scrambled is a control oligo with a scrambled sequence not related to the Htra1 target sequence.

FIGS. 5A-5G. NHP PK/PD study, IVT administration, 25 μg/eye. A) HTRA1 mRNA level measured in the retina by qPCR. B) oligo content in the retina measured by oligo ELISA. C) HTRA1 mRNA level illustrated by ISH. D-E) Quantification of HTRA1 protein level in retina and vitreous, respectively, by IP-MS. Dots show data for individual animals. Error bars show standard errors for technical replicates (n=3). F-G) Reduction in HTRA1 protein level in retina and vitreous, respectively illustrated by western blot.

FIG. 6. A Compound of the invention (Compound ID NO 67,1). The compound may be in the form of a pharmaceutical salt, such as a sodium salt or a potassium salt.

FIG. 7. A Compound of the invention (Compound ID NO 86,1). The compound may be in the form of a pharmaceutical salt, such as a sodium salt or a potassium salt.

FIG. 8. A Compound of the invention (Compound ID NO 73,1). The compound may be in the form of a pharmaceutical salt, such as a sodium salt or a potassium salt.

FIG. 9. An example of a pharmaceutical salt of compound 67,1: M+ is a suitable cation, typically a positive metal ion, such as a sodium or potassium ion. The stoichiometric ratio of the cation to the oligonucleotide anion will depend on the charge of the cation used. Suitably, cations with one, two or three positive charge (M⁺, M⁺⁺, or M⁺⁺⁺, may be used). For illustrative purpose, twice as many single+charged cations (monovalent), such as Na⁺ or K⁺ are needed as compared to a divalent cation such as Ca²⁺.

FIG. 10. An example of a pharmaceutical salt of compound 86,1: See the figure legend for FIG. 9 for the description of the cation M.

FIG. 11. An example of a pharmaceutical salt of compound 73,1: See the figure legend for FIG. 9 for the description of the cation M.

FIG. 12A. Compounds #15,3 and #17 were administered intravitreally in cynomolgus monkeys, and aqueous humor samples were collected at days 3, 8, 15, and 22 post-injection. Proteins from undiluted samples were analyzed by capillary electrophoresis using a Peggy Sue device (Protein Simple). HTRA1 was detected using a custom-made polycolonal rabbit antiserum. Data from animals #J60154 (Vehicle), J60158 (C. Id#15,3), J60162 (C. Id#17) are presented.

FIG. 12B. Signal intensities were quantified by comparison to purified recombinant (S328A mutant) HTRA1 protein (Origene, #TP700208). The calibration curve is shown here.

FIG. 12C. Top panel: Calculated HTRA1 aqueous humor concentration from individual animal was plotted against time post injection. Bottom panel: average HTRA1 concentration for the vehicle group at each time point was determined and corresponding relative concentration in treated animals calculated. Open circle: individual value, closed circle: group average. HTRA1 reduction for day 22 is indicated.

FIG. 13. HTRA1 mRNA plotted against HTRA1 protein levels in aqueous humor (blue diamonds) or in retina (red squares) in cynomolgus monkeys treated with various LNA molecules targeting the HTRA1 transcript. Values are expressed as percentage normalized to PBS controls.

FIG. 14. Correlation of HTRA1 protein in aqueous humor with (A) HTRA1 protein in retina and (B) HTRA1 mRNA in retina in cynomolgus monkeys treated with various LNA molecules targeting the HTRA1 transcript. Values are expressed as percentage normalized to PBS controls.

### DEFINITIONS

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

### Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs. Preferably, the antisense oligonucleotides of the present invention are single stranded.

### Contiguous Nucleotide Region

The term “contiguous nucleotide region” refers to the region of the oligonucleotide which is complementary to the target nucleic acid. The term may be used interchangeably herein with the term “contiguous nucleotide sequence” or “contiguous nucleobase sequence” and the term “oligonucleotide motif sequence”. In some embodiments all the nucleotides of the oligonucleotide are present in the contiguous nucleotide region. In some embodiments the oligonucleotide comprises the contiguous nucleotide region and may, optionally comprise further nucleotide(s), for example a nucleotide linker region which may be used to attach a functional group to the contiguous nucleotide sequence. The nucleotide linker region may or may not be complementary to the target nucleic acid. In some embodiments the internucleoside linkages present between the nucleotides of the contiguous nucleotide region are all phosphorothioate internucleoside linkages. In some embodiments, the contiguous nucleotide region comprises one or more sugar modified nucleosides.

### Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”.

### Modified Internucleoside Linkage

The term “modified internucleoside linkage” is defined as generally understood by the skilled person as linkages other than phosphodiester (PO) linkages, that covalently couples two nucleosides together. Nucleotides with modified internucleoside linkage are also termed “modified nucleotides”. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides.

In an embodiment, the oligonucleotide comprises one or more internucleoside linkages modified from the natural phosphodiester to a linkage that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

In some embodiments the modified internucleoside linkages may be phosphorothioate internucleoside linkages. In some embodiments, the modified internucleoside linkages are compatible with the RNaseH recruitment of the oligonucleotide of the invention, for example phosphorothioate.

In some embodiments the internucleoside linkage comprises sulphur (S), such as a phosphorothioate internucleoside linkage.

A phosphorothioate internucleoside linkage is particularly useful due to nuclease resistance, beneficial pharmakokinetics and ease of manufacture. In some embodiments all of the internucleoside linkages of the oligonucleotide, or contiguous nucleotide sequence thereof, are phosphorothioate.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used. In some embodiments, the cytosine nucleobases in a 5′cg3′ motif is 5-methyl cytosine.

### Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

### Complementarity

The term complementarity describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide region or sequence in a nucleic acid molecule (e.g. oligonucleotide) which, at a given position, are complementary to (i.e. form Watson Crick base pairs with) a contiguous nucleotide sequence, at a given position of a separate nucleic acid molecule (e.g. the target nucleic acid). The percentage is calculated by counting the number of aligned bases that form pairs between the two sequences, dividing by the total number of nucleotides in the oligonucleotide and multiplying by 100. In such a comparison a nucleobase/nucleotide which does not align (form a base pair) is termed a mismatch.

It will be understood that when referring to complementarity between two sequences, the determination of complementarity is measured across the length of the shorter of the two sequences, such as the length of the contiguous nucleotide region or sequence.

The term “fully complementary”, refers to 100% complementarity. In the absence of a % term value or indication of a mismatch, complementary means fully complementary.

### Identity

The term “Identity” as used herein, refers to the number of nucleotides in percent of a contiguous nucleotide sequence in a nucleic acid molecule (e.g. oligonucleotide) which, at a given position, are identical to (i.e. in their ability to form Watson Crick base pairs with the complementary nucleoside) a contiguous nucleotide sequence, at a given position of a separate nucleic acid molecule (e.g. the target nucleic acid). The percentage is calculated by counting the number of aligned bases that are identical between the two sequences, including gaps, dividing by the total number of nucleotides in the oligonucleotide and multiplying by 100. Percent Identity=(Matches×100)/Length of aligned region (with gaps).

When determining the identity of the contiguous nucleotide region of an oligonucleotide, the identity is calculated across the length of the contiguous nucleotide region. In embodiments where the entire contiguous nucleotide sequence of the oligonucleotide is the contiguous nucleotide region, identity is therefore calculated across the length of the nucleotide sequence of the oligonucleotide. In this respect the contiguous nucleotide region may be identical to a region of the reference nucleic acid sequence, or in some embodiments may be identical to the entire reference nucleic acid. Unless otherwise indicated a sequence which has 100% identity to a reference sequence is referred to as being identical.

For example, the reference sequence may be selected from the group consisting of any one of SEQ ID NOs 5-111.

However, if the oligonucleotide comprises additional nucleotide(s) flanking the contiguous nucleotide region, for example region D′ or D″, these additional flanking nucleotides may be disregarded when determining identity. In some embodiments, identity may be calculated across the entire oligonucleotide sequence.

In some embodiments, the antisense oligonucleotide oligonucleotide of the invention comprises a contiguous nucleotide region of at least 10 contiguous nucleotides which are identical to a sequence selected from the group consisting of SEQ ID NO 5-111.

In some embodiments, the antisense oligonucleotide oligonucleotide of the invention comprises a contiguous nucleotide region of at least 12 contiguous nucleotides which are identical to a sequence selected from the group consisting of SEQ ID NO 5-111.

In some embodiments, the antisense oligonucleotide oligonucleotide of the invention comprises a contiguous nucleotide region of at least 13 contiguous nucleotides which are identical to a sequence selected from the group consisting of SEQ ID NO 5-111.

In some embodiments, the antisense oligonucleotide oligonucleotide of the invention comprises a contiguous nucleotide region of at least 14 contiguous nucleotides which are identical to a sequence selected from the group consisting of SEQ ID NO 5-111.

In some embodiments, the antisense oligonucleotide oligonucleotide of the invention comprises a contiguous nucleotide region of at least 15 contiguous nucleotides which are identical to a sequence selected from the group consisting of SEQ ID NO 5-111.

In some embodiments, the antisense oligonucleotide oligonucleotide of the invention comprises a contiguous nucleotide region of at least 16 contiguous nucleotides which are identical to a sequence selected from the group consisting of SEQ ID NO 5-111.

In some embodiments, the contiguous nucleotide region consists or comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides of a sequence selected form the group consisting of SEQ ID NO 113-118, or SEQ ID NO 5-111 . . . In some embodiments, the entire contiguous sequence of the oligonucleotide consists or comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides of SEQ ID NO

In some embodiments, the contiguous sequence of the oligonucleotide consists or comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides of SEQ ID NO 119.

In some embodiments, the contiguous sequence of the oligonucleotide consists or comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides of SEQ ID NO 120.

In some embodiments, the contiguous sequence of the oligonucleotide consists or comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides of SEQ ID NO 121.

In some embodiments, the contiguous sequence of the oligonucleotide consists or comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides of SEQ ID NO 122.

In some embodiments, the contiguous sequence of the oligonucleotide consists or comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides of SEQ ID NO 123.

The invention provides an antisense oligonucleotide which comprises a contiguous nucleotide region of at least 10, or at least 12, or at least 13, or at least 14 or at least 15 or at least 16 or at least 17 or at least 18 contiguous nucleotides present SEQ ID NO 118: 5′ CTTCTTCTATCTACGCATTG 3′.

In some embodiments, the contiguous nucleotide region comprises 10, 11, 12, 13, 14, 15 or 16 contiguous nucleotides which are identical to SEQ ID NO 67.

In some embodiments, the contiguous nucleotide region comprises 10, 11, 12, 13, 14, 15, 16, 17 or 18 contiguous nucleotides which are identical to SEQ ID NO 73.

In some embodiments, the contiguous nucleotide region comprises 10, 11, 12, 13, 14, 15 or 16 contiguous nucleotides which are identical to SEQ ID NO 86.

The invention provides for an antisense oligonucleotide 11-30 nucleotides in length, such as 12-20 nucleotides in length, wherein the oligonucleotide comprises a contiguous nucleotide sequence identical to a sequence selected from the group consisting of SEQ ID NO 5-111. The invention provides for an antisense oligonucleotide comprising or consisting of a contiguous nucleotide sequence, wherein the contiguous nucleotide sequence is identical to a reference sequence selected from the group consisting of SEQ ID NO 5-111 across at least 10 contiguous nucleotide of the reference sequence.

The invention provides for an antisense oligonucleotide comprising or consisting of a contiguous nucleotide sequence, wherein the contiguous nucleotide sequence is identical to a reference sequence selected from the group consisting of SEQ ID NO 5-111 across at least 12 contiguous nucleotide of the reference sequence.

The invention provides for an antisense oligonucleotide comprising or consisting of a contiguous nucleotide sequence, wherein the contiguous nucleotide sequence is identical to a reference sequence selected from the group consisting of SEQ ID NO 5-111 across at least 14 contiguous nucleotide of the reference sequence.

The invention provides for an antisense oligonucleotide comprising or consisting of a contiguous nucleotide sequence, wherein the contiguous nucleotide sequence is identical to a reference sequence selected from the group consisting of SEQ ID NO 5-111 across the length of the reference sequence.

### Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Sequence

The oligonucleotide comprises a contiguous nucleotide region which is complementary to or hybridizes to a sub-sequence of the target nucleic acid molecule. The term “target sequence” as used herein refers to a sequence of nucleotides present in the target nucleic acid which comprises the nucleobase sequence which is complementary to the contiguous nucleotide region or sequence of the oligonucleotide of the invention. In some embodiments, the target sequence consists of a region on the target nucleic acid which is complementary to the contiguous nucleotide region or sequence of the oligonucleotide of the invention. In some embodiments the target sequence is longer than the complementary sequence of a single oligonucleotide, and may, for example represent a preferred region of the target nucleic acid which may be targeted by several oligonucleotides of the invention.

The oligonucleotide of the invention comprises a contiguous nucleotide region which is complementary to the target nucleic acid, such as a target sequence.

The oligonucleotide comprises a contiguous nucleotide region of at least 10 nucleotides which is complementary to or hybridizes to a target sequence present in the target nucleic acid molecule. The contiguous nucleotide region (and therefore the target sequence) comprises of at least 10 contiguous nucleotides, such as 11, 12, 13, 14, 15, 16, 17, 18, 19, 20, 21, 22, contiguous nucleotides, such as from 12-22, such as from 14-18 contiguous nucleotides.

In some embodiments the target sequence is present within a sequence selected from the group consisting of SEQ ID NO 113, 114, 115, 116, 117 and 118.

Target Cell

The term a target cell as used herein refers to a cell which is expressing the target nucleic acid. In some embodiments the target cell may be in vivo or in vitro. In some embodiments the target cell is a mammalian cell such as a primate cell such as a monkey cell or a human cell. In some embodiments the target cell may be a retinal cell, such as a retinal pigment epithelium (PRE) cell. In some embodiments the cell is selected from the group consisting of RPE cells, Bipolar Cell, Amacrine cells, Endothelial cells, Ganglion cells and Microglia cells. For in vitro assessment, the target cell may be a primary cell or an established cell line, such as U251, ARPE19 . . . .

### Target Nucleic Acid

According to the present invention, the target nucleic acid is a nucleic acid which encodes mammalian HTRA1 and may for example be a gene, a RNA, a mRNA, and pre-mRNA, a mature mRNA or a cDNA sequence. The target may therefore be referred to as an HTRA1 target nucleic acid.

Suitably, the target nucleic acid encodes an HTRA1 protein, in particular mammalian HTRA1, such as human HTRA1 (See for example tables 1 & 2 which provides the mRNA and pre-mRNA sequences for human and rat HTRA1).

In some embodiments, the target nucleic acid is selected from the group consisting of SEQ ID NO: 1, 2, 3, and 4, or naturally occurring variants thereof (e.g. sequences encoding a mammalian HTRA1 protein.

A target cell is a cell which is expressing the HTRA1 target nucleic acid. In preferred embodiments the target nucleic acid is the HTRA1 mRNA, such as the HTRA1 pre-mRNA or HTRA1 mature mRNA. The poly A tail of HTRA1 mRNA is typically disregarded for antisense oligonucleotide targeting.

If employing the oligonucleotide of the invention in research or diagnostics the target nucleic acid may be a cDNA or a synthetic nucleic acid derived from DNA or RNA.

The target sequence may be a sub-sequence of the target nucleic acid. In some embodiments the oligonucleotide or contiguous nucleotide region is fully complementary to, or only comprises one or two mismatches to an HTRA1 sub-sequence, such as a sequence selected from the group consisting of SEQ ID NO 113, 114, 115, 116, 117 or 231.

The target sequence may be a sub-sequence of the target nucleic acid. In some embodiments the oligonucleotide or contiguous nucleotide region is fully complementary to, or only comprises one or two mismatches to an HTRA1 sub-sequence, such as a sequence selected from the group consisting of SEQ ID NO 124-230. In some embodiments the oligonucleotide or contiguous nucleotide region is fully complementary to, or only comprises one or two mismatches to an HTRA1 sub-sequence SEQ ID NO 231.

Complementarity to the target or sub-sequence thereof is measured over the length of the oligonucleotide, or contiguous nucleotide region thereof.

For in vivo or in vitro application, the oligonucleotide of the invention is typically capable of inhibiting the expression of the HTRA1 target nucleic acid in a cell which is expressing the HTRA1 target nucleic acid. The contiguous sequence of nucleobases of the oligonucleotide of the invention is typically complementary to the HTRA1 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D). The target nucleic acid may, in some embodiments, be a RNA or DNA, such as a messenger RNA, such as a mature mRNA or a pre-mRNA. In some embodiments the target nucleic acid is a RNA or DNA which encodes mammalian HTRA1 protein, such as human HTRA1, e.g. the human HTRA1 mRNA sequence, such as that disclosed as SEQ ID NO 1 (NM_002775.4, GI:190014575). Further information on exemplary target nucleic acids is provided in tables 1 & 2.

TABLE 1
Genome and assembly information for human and Cyno HTRA1.

| | | | | | | NCBI reference |
|---|---|---|---|---|---|---|
| | | | Genomic coordinates | | | sequence* accession |
| Species | Chr. | Strand | Start | End | Assembly | number for mRNA |
| Human | 10 | fwd | 122461525 | 122514908 | GRCh38.p2 release 107 | NM_002775.4 |
| Cyno | 9 | fwd | 121764994 | 121817518 | Macaca_fascicularis_5.0 | NC_022280.1** |

Fwd = forward strand. The genome coordinates provide the pre-mRNA sequence (genomic sequence).
The NCBI reference provides the mRNA sequence (cDNA sequence).
*The National Center for Biotechnology Information reference sequence database is a comprehensive, integrated, non-redundant, well-annotated set of reference sequences including genomic, transcript, and protein. It is hosted at www.ncbi.nlm.nih.gov/refseq.
**In the NCBI reference sequence there is a stretch of 100 nucleotides from position 126 to position 227 whose identity is not known. In SEQ ID NO 3 & 4, this stretch has been replaced by the nucleotides appearing in both human and Macaca mulatta HTRA1 premRNA sequences in this region.

TABLE 2
Sequence details for human and Cyno HTRA1.

| | | Length | SEQ ID |
|---|---|---|---|
| Species | RNA type | (nt) | NO |
| Human | mRNA | 2138 | 1 |
| Human | premRNA | 53384 | 2 |
| Cyno | mRNA | 2123 | 3 |
| Cyno | premRNA | 52575 | 4 |

Naturally Occurring Variant The term “naturally occurring variant” refers to variants of HTRA1 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms, and allelic variants. Based on the presence of the sufficient complementary sequence to the oligonucleotide, the oligonucleotide of the invention may therefore target the target nucleic acid and naturally occurring variants thereof. In some embodiments, the naturally occurring variants have at least 95% such as at least 98% or at least 99% homology to a mammalian HTRA1 target nucleic acid, such as a target nucleic acid selected form the group consisting of SEQ ID NO 1, 2, 3, or 4.

### Modulation of Expression

The term “modulation of expression” as used herein is to be understood as an overall term for an oligonucleotide's ability to alter the amount of HTRA1 when compared to the amount of HTRA1 before administration of the oligonucleotide. Alternatively modulation of expression may be determined by reference to a control experiment where the oligonucleotide of the invention is not administered. One type of modulation is an oligonucleotide's ability to inhibit, down-regulate, reduce, suppress, remove, stop, block, prevent, lessen, lower, avoid or terminate expression of HTRA1, e.g. by degradation of mRNA or blockage of transcription. The antisense oligonucleotide of the invention are capable of inhibiting, down-regulating, reduce, suppress, remove, stop, block, prevent, lessen, lower, avoid or terminate expression of HTRA1.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

### 2′ Modified Nucleosides.

A 2′ sugar modified nucleoside is a nucleoside which has a substituent other than H or —OH at the 2′ position (2′ substituted nucleoside) or comprises a 2′ linked biradicle, and includes 2′ substituted nucleosides and LNA (2′-4′ biradicle bridged) nucleosides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

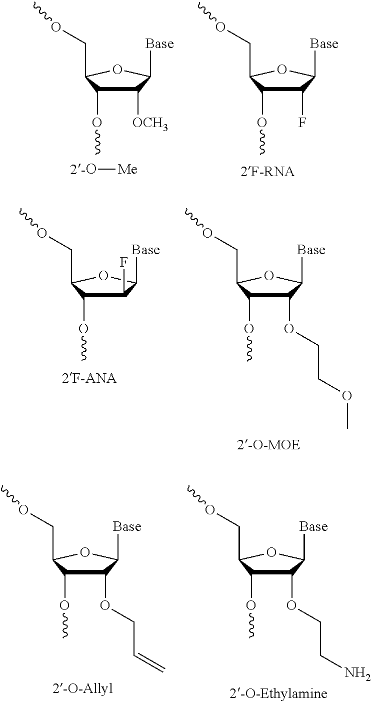

### Locked Nucleic Acid Nucleosides (LNA).

LNA nucleosides are modified nucleosides which comprise a linker group (referred to as a biradicle or a bridge) between C2′ and C4′ of the ribose sugar ring of a nucleotide. These nucleosides are also termed bridged nucleic acid or bicyclic nucleic acid (BNA) in the literature.

In some embodiments, the modified nucleoside or the LNA nucleosides of the oligomer of the invention has a general structure of the formula I or II:

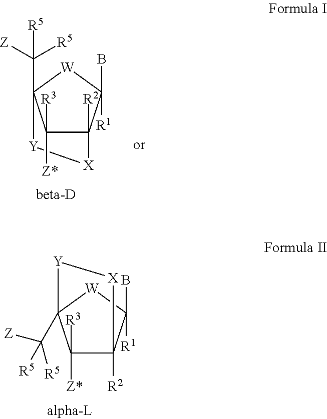

wherein W is selected from —O—, —S—, —N(R^{a})—, —C(R^{a}R^{b})—, such as, in some embodiments —O—;
B designates a nucleobase moiety;
Z designates an internucleoside linkage to an adjacent nucleoside, or a 5′-terminal group;
Z* designates an internucleoside linkage to an adjacent nucleoside, or a 3′-terminal group;
X designates a group selected from the list consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z

In some embodiments, X is selected from the group consisting of: —O—, —S—, NH—, NR^{a}R^{b}, —CH₂—, CR^{a}R^{b}, —C(═CH₂)—, and —C(═CR^{a}R^{b})—
In some embodiments, X is —O—

Y designates a group selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z

In some embodiments, Y is selected from the group consisting of: —CH₂—, —C(R^{a}R^{b})—, —CH₂CH₂—, —C(R^{a}R^{b})—C(R^{a}R^{b})—, —CH₂CH₂CH₂—, —C(R^{a}R^{b})C(R^{a}R^{b})C(R^{a}R^{b})—, —C(R^{a})═C(R^{b})—, and —C(R^{a})═N—

In some embodiments, Y is selected from the group consisting of: —CH₂—, —CHR^{a}—, —CHCH₃—, CR^{a}R^{b}—

or —X—Y— together designate a bivalent linker group (also referred to as a radical) together designate a bivalent linker group consisting of 1, 2, or 3 groups/atoms selected from the group consisting of —C(R^{a}R^{b})—, —C(R^{a})═C(R^{b})—, —C(R^{a})═N—, —O—, —Si(R^{a})₂—, —S—, —SO₂—, —N(R^{a})—, and >C═Z,

In some embodiments, —X—Y— designates a biradicle selected from the groups consisting of: —X—CH₂—, —X—CR^{a}R^{b}—, —X—CHR^{a-}, —O—Y—, —O—CH₂—, —S—CH₂—, —NH—CH₂—, —O—CHCH₃—, —CH₂—O—CH₂, —O—CH(CH₃CH₃)—, —O—CH₂—CH₂—, OCH₂—CH₂—CH₂—, —O—CH₂OCH₂—, —O—NCH₂—, —C(═CH₂)—CH₂—, —NR^{a}—CH₂—, N—O—CH₂, —S—CR^{a}R^{b}— and —S—CHR^{a}—.
In some embodiments —X—Y— designates —O—CH₂— or —O—CH(CH₃)—.

wherein Z is selected from —O—, —S—, and —N(R^{a})—, and R^{a} and, when present R^{b}, each is independently selected from hydrogen, optionally substituted C₁₋₆-alkyl, optionally substituted C₂₋₆-alkenyl, optionally substituted C₂₋₆-alkynyl, hydroxy, optionally substituted C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted and where two geminal substituents R^{a} and R^{b} together may designate optionally substituted methylene (═CH₂), wherein for all chiral centers, asymmetric groups may be found in either R or S orientation.

wherein R¹, R², R³, R⁵ and R^{5*} are independently selected from the group consisting of: hydrogen, optionally substituted C₁₋₆-alkyl, optionally substituted C₂₋₆-alkenyl, optionally substituted C₂₋₆-alkynyl, hydroxy, C₁₋₆-alkoxy, C₂₋₆-alkoxyalkyl, C₂₋₆-alkenyloxy, carboxy, C₁₋₆-alkoxycarbonyl, C₁₋₆-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, where aryl and heteroaryl may be optionally substituted, and where two geminal substituents together may designate oxo, thioxo, imino, or optionally substituted methylene.

In some embodiments R¹, R², R³, R⁵ and R^{5*} are independently selected from C₁₋₆ alkyl, such as methyl, and hydrogen.
In some embodiments R¹, R², R³, R⁵ and R^{5*} are all hydrogen.

In some embodiments R¹, R², R³, are all hydrogen, and either R⁵ and R^{5*} is also hydrogen and the other of R⁵ and R^{5*}is other than hydrogen, such as C₁₋₆ alkyl such as methyl.

In some embodiments, R^{a} is either hydrogen or methyl. In some embodiments, when present, R^{b} is either hydrogen or methyl.

In some embodiments, one or both of R^{a} and R^{b} is hydrogen

In some embodiments, one of R^{a} and R^{b} is hydrogen and the other is other than hydrogen
In some embodiments, one of R^{a} and R^{b} is methyl and the other is hydrogen
In some embodiments, both of R^{a} and R^{b} are methyl.

In some embodiments, the biradicle —X—Y— is —O—CH₂—, W is O, and all of R¹, R², R³, R⁵ and R^{5*} are all hydrogen. Such LNA nucleosides are disclosed in WO99/014226, WO00/66604, WO98/039352 and WO2004/046160 which are all hereby incorporated by reference, and include what are commonly known as beta-D-oxy LNA and alpha-L-oxy LNA nucleosides.

In some embodiments, the biradicle —X—Y— is —S—CH₂—, W is O, and all of R¹, R², R³, R⁵ and R^{5*} are all hydrogen. Such thio LNA nucleosides are disclosed in WO99/014226 and WO2004/046160 which are hereby incorporated by reference.

In some embodiments, the biradicle —X—Y— is —NH—CH₂—, W is O, and all of R¹, R², R³, R⁵ and R^{5*} are all hydrogen. Such amino LNA nucleosides are disclosed in WO99/014226 and WO2004/046160 which are hereby incorporated by reference.

In some embodiments, the biradicle —X—Y— is —O—CH₂—CH₂— or —O—CH₂—CH₂— CH₂—, W is O, and all of R¹, R², R³, R⁵ and R^{5*} are all hydrogen. Such LNA nucleosides are disclosed in WO00/047599 and Morita et al, Bioorganic & Med. Chem. Lett. 12 73-76, which are hereby incorporated by reference, and include what are commonly known as 2′-O-4′C-ethylene bridged nucleic acids (ENA).

In some embodiments, the biradicle —X—Y— is —O—CH₂—, W is O, and all of R¹, R², R³, and one of R⁵ and R^{5*} are hydrogen, and the other of R⁵ and R^{5*} is other than hydrogen such as C₁₋₆ alkyl, such as methyl. Such 5′ substituted LNA nucleosides are disclosed in WO2007/134181 which is hereby incorporated by reference.

In some embodiments, the biradicle —X—Y— is —O—CR^{a}R^{b}—, wherein one or both of R^{a} and R^{b} are other than hydrogen, such as methyl, W is O, and all of R¹, R², R³, and one of R⁵ and R^{5*} are hydrogen, and the other of R⁵ and R^{5*} is other than hydrogen such as C₁₋₆ alkyl, such as methyl. Such bis modified LNA nucleosides are disclosed in WO2010/077578 which is hereby incorporated by reference.

In some embodiments, the biradicle —X—Y— designate the bivalent linker group —O—CH(CH₂OCH₃)— (2′ O-methoxyethyl bicyclic nucleic acid—Seth at al., 2010, J. Org. Chem. Vol 75(5) pp. 1569-81). In some embodiments, the biradicle —X—Y— designate the bivalent linker group —O—CH(CH₂CH₃)— (2′O-ethyl bicyclic nucleic acid—Seth at al., 2010, J. Org. Chem. Vol 75(5) pp. 1569-81). In some embodiments, the biradicle —X—Y— is —O—CHR^{a}—, W is O, and all of R¹, R², R³, R⁵ and R^{5*} are all hydrogen. Such 6′ substituted LNA nucleosides are disclosed in WO10036698 and WO07090071 which are both hereby incorporated by reference.

In some embodiments, the biradicle —X—Y— is —O—CH(CH₂OCH₃)—, W is O, and all of R¹, R², R³, R⁵ and R^{5*} are all hydrogen. Such LNA nucleosides are also known as cyclic MOEs in the art (cMOE) and are disclosed in WO07090071.

In some embodiments, the biradicle —X—Y— designate the bivalent linker group —O—CH(CH₃)—.— in either the R- or S-configuration. In some embodiments, the biradicle —X—Y— together designate the bivalent linker group —O—CH₂—O—CH₂— (Seth at al., 2010, J. Org. Chem). In some embodiments, the biradicle —X—Y— is —O—CH(CH₃)—, W is O, and all of R¹, R², R³, R⁵ and R^{5*} are all hydrogen. Such 6′ methyl LNA nucleosides are also known as cET nucleosides in the art, and may be either (S)cET or (R)cET stereoisomers, as disclosed in WO07090071 (beta-D) and WO2010/036698 (alpha-L) which are both hereby incorporated by reference).

In some embodiments, the biradicle —X—Y— is —O—CR^{a}R^{b}—, wherein in neither R^{a} or R^{b} is hydrogen, W is O, and all of R¹, R², R³, R⁵ and R^{5*} are all hydrogen. In some embodiments, R^{a} and R^{b} are both methyl. Such 6′ di-substituted LNA nucleosides are disclosed in WO 2009006478 which is hereby incorporated by reference.

In some embodiments, the biradicle —X—Y— is —S—CHR^{a}—, W is O, and all of R¹, R², R³, R⁵ and R^{5*} are all hydrogen. Such 6′ substituted thio LNA nucleosides are disclosed in WO11156202 which is hereby incorporated by reference. In some 6′ substituted thio LNA embodiments R^{a} is methyl.

In some embodiments, the biradicle —X—Y— is —C(═CH₂)—C(R^{a}R^{b})—, such as —C(═CH₂)—CH₂—, or —C(═CH₂)—CH(CH₃)—W is O, and all of R¹, R², R³, R⁵ and R^{5*} are all hydrogen. Such vinyl carbo LNA nucleosides are disclosed in WO08154401 and WO09067647 which are both hereby incorporated by reference.

In some embodiments the biradicle —X—Y— is —N(—OR^{a})—, W is O, and all of R¹, R², R³, R⁵ and R^{5*} are all hydrogen. In some embodiments R^{a} is C₁₋₆ alkyl such as methyl. Such LNA nucleosides are also known as N substituted LNAs and are disclosed in WO2008/150729 which is hereby incorporated by reference. In some embodiments, the biradicle —X—Y— together designate the bivalent linker group —O—NR^{a}—CH₃— (Seth at al., 2010, J. Org. Chem). In some embodiments the biradicle —X—Y— is —N(R^{a})—, W is O, and all of R¹, R², R³, R⁵ and R^{5*} are all hydrogen. In some embodiments R^{a} is C₁₋₆ alkyl such as methyl.

In some embodiments, one or both of R⁵ and R^{5*} is hydrogen and, when substituted the other of R⁵ and R^{5*} is C₁₋₆ alkyl such as methyl. In such an embodiment, R¹, R², R³, may all be hydrogen, and the biradicle —X—Y— may be selected from —O—CH2— or —O—C(HCR^{a})—, such as —O—C(HCH3)—.

In some embodiments, the biradicle is —CR^{a}R^{b}—O—CR^{a}R^{b}—, such as CH₂—O—CH₂—, W is O and all of R¹, R², R³, R⁵ and R^{5*} are all hydrogen. In some embodiments R^{a} is C1-6 alkyl such as methyl. Such LNA nucleosides are also known as conformationally restricted nucleotides (CRNs) and are disclosed in WO2013036868 which is hereby incorporated by reference.

In some embodiments, the biradicle is —O—CR^{a}R^{b}—O—CR^{a}R^{b}—, such as O—CH₂—O—CH₂—, W is O and all of R¹, R², R³, R⁵ and R^{5*} are all hydrogen. In some embodiments R^{a} is C₁₋₆alkyl such as methyl. Such LNA nucleosides are also known as COC nucleotides and are disclosed in Mitsuoka et al., Nucleic Acids Research 2009 37(4), 1225-1238, which is hereby incorporated by reference.

It will be recognized than, unless specified, the LNA nucleosides may be in the beta-D or alpha-L stereoisoform.

Examples of LNA nucleosides are presented in Scheme 1.

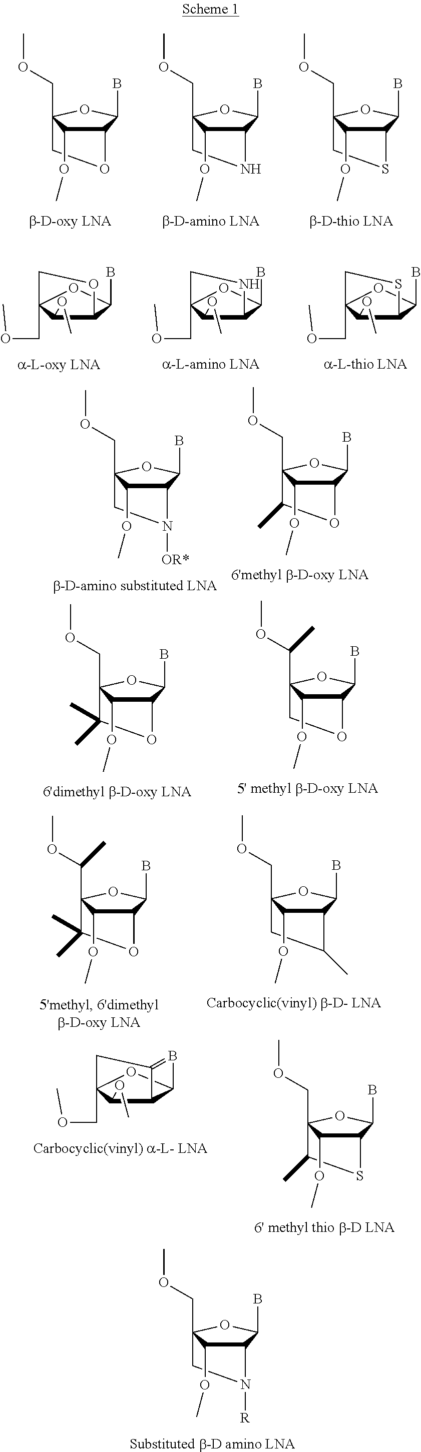

As illustrated in the examples, in some embodiments of the invention the LNA nucleosides in the oligonucleotides are beta-D-oxy-LNA nucleosides.

### Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers, headmers and tailmers.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers, with phosphorothioate linkages between all monomers in the oligonucleotide, and using the methodology provided by Example 91-95 of WO01/23613 (hereby incorporated by reference).

### Gapmer

The term gapmer as used herein refers to an antisense oligonucleotide which comprises a region of RNase H recruiting oligonucleotides (gap) which is flanked 5′ and 3′ by regions which comprise one or more affinity enhancing modified nucleosides (flanks or wings). Various gapmer designs are described herein. Headmers and tailmers are oligonucleotides capable of recruiting RNase H where one of the flanks is missing, i.e. only one of the ends of the oligonucleotide comprises affinity enhancing modified nucleosides. For headmers the 3′ flank is missing (i.e. the 5′ flank comprises affinity enhancing modified nucleosides) and for tailmers the 5′ flank is missing (i.e. the 3′ flank comprises affinity enhancing modified nucleosides).

### LNA Gapmer

The term LNA gapmer is a gapmer oligonucleotide wherein at least one of the affinity enhancing modified nucleosides is an LNA nucleoside. In some embodiments the LNA nucleoside(s) in an LNA gapmer are beta-D-oxy LNA nucleosides and/or 6′methyl beta-D-oxy LNA nucleosides (such as (S)cET nucleosides.

### Mixed Wing Gapmer

The term mixed wing gapmer refers to a LNA gapmer wherein the flank regions comprise at least one LNA nucleoside and at least one non-LNA modified nucleoside, such as at least one DNA nucleoside or at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA and 2′-F-ANA nucleoside(s). In some embodiments the mixed wing gapmer has one flank which comprises LNA nucleosides (e.g. 5′ or 3′) and the other flank (3′ or 5′ respectfully) comprises 2′ substituted modified nucleoside(s). In some embodiments the LNA nucleoside(s) in an mixed wing gapmer are beta-D-oxy LNA nucleosides and/or 6′methyl beta-D-oxy LNA nucleosides (such as (S)cET nucleosides.

### Conjugate

The term conjugate as used herein refers to an oligonucleotide which is covalently linked to a non-nucleotide moiety (conjugate moiety or region C or third region).

The term conjugate as used herein refers to an oligonucleotide which is covalently linked to a non-nucleotide moiety (conjugate moiety or region C or third region).

In some embodiments, the non-nucleotide moiety selected from the group consisting of a protein, such as an enzyme, an antibody or an antibody fragment or a peptide; a lipophilic moiety such as a lipid, a phospholipid, a sterol; a polymer, such as polyethyleneglycol or polypropylene glycol; a receptor ligand; a small molecule; a reporter molecule; and a non-nucleosidic carbohydrate.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C).

In some embodiments of the invention the conjugate or oligonucleotide conjugate of the invention may optionally, comprise a linker region which is positioned between the oligonucleotide and the conjugate moiety. In some embodiments, the linker between the conjugate and oligonucleotide is biocleavable.

Biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference), and may be referred to as region D herein.

Conjugates may also be linked to the oligonucleotide via non biocleavable linkers, or in some embodiments the conjugate may comprise a non-cleavable linker which is covalently attached to the biocleavable linker. Linkers that are not necessarily biocleavable but primarily serve to covalently connect a conjugate moiety to an oligonucleotide or biocleavable linker. Such linkers may comprise a chain structure or an oligomer of repeating units such as ethylene glycol, amino acid units or amino alkyl groups. In some embodiments the linker (region Y) is an amino alkyl, such as a C₂-C₃₆ amino alkyl group, including, for example C₆ to C₁₂ amino alkyl groups. In some embodiments the linker (region Y) is a C₆ amino alkyl group. Conjugate linker groups may be routinely attached to an oligonucleotide via use of an amino modified oligonucleotide, and an activated ester group on the conjugate group.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

### The Oligonucleotides of the Invention

The invention relates to oligonucleotides capable of inhibiting the expression of HTRA1. The modulation is may achieved by hybridizing to a target nucleic acid encoding HTRA1 or which is involved in the regulation of HTRA1. The target nucleic acid may be a mammalian HTRA 1 sequence, such as a sequence selected from the group consisting of SEQ ID 1, 2, 3 or 4.

The oligonucleotide of the invention is an antisense oligonucleotide which targets HTRA1, such as a mammalian HTRA1.

In some embodiments the antisense oligonucleotide of the invention is capable of modulating the expression of the target by inhibiting or down-regulating it. Preferably, such modulation produces an inhibition of expression of at least 20% compared to the normal expression level of the target, such as at least 30%, 40%, 50%, 60%, 70%, 80%, or 90% inhibition compared to the normal expression level of the target. In some embodiments compounds of the invention may be capable of inhibiting expression levels of HTRA1 mRNA by at least 60% or 70% in vitro using ARPE-19 cells. In some embodiments compounds of the invention may be capable of inhibiting expression levels of HTRA1 mRNA by at least 60% or 70% in vitro using ARPE-19 cells. In some embodiments compounds of the invention may be capable of inhibiting expression levels of HTRA1 protein by at least 50% in vitro using ARPE-19 cells. Suitably, the examples provide assays which may be used to measure HTRA1 RNA or protein inhibition. The target modulation is triggered by the hybridization between a contiguous nucleotide sequence of the oligonucleotide and the target nucleic acid. In some embodiments the oligonucleotide of the invention comprises mismatches between the oligonucleotide and the target nucleic acid. Despite mismatches hybridization to the target nucleic acid may still be sufficient to show a desired modulation of HTRA1 expression. Reduced binding affinity resulting from mismatches may advantageously be compensated by increased number of nucleotides in the oligonucleotide and/or an increased number of modified nucleosides capable of increasing the binding affinity to the target, such as 2′ modified nucleosides, including LNA, present within the oligonucleotide sequence.

An aspect of the present invention relates to an antisense oligonucleotide which comprises a contiguous nucleotide region of 10 to 30 nucleotides in length with at least 90% complementarity to HTRA1 target sequence, such as fully complementary to an HTRA1 target sequence, e.g. a nucleic acid selected from the group consisting SEQ ID NO 1, 2, 3 & 4.

In some embodiments, the oligonucleotide comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid.

In some embodiments, the oligonucleotide of the invention, or a contiguous nucleotide sequence thereof is fully complementary (100% complementary) to a region of the target nucleic acid, or in some embodiments may comprise one or two mismatches between the oligonucleotide and the target nucleic acid.

In some embodiments the oligonucleotide, or a contiguous nucleotide sequence of at least 12 nucleotides thereof, is at least 90% complementary, such as fully (or 100%) complementary to a region of a sequence selected from the group consisting of SEQ ID NO 119, 120, 121, 122 or 123.

In some embodiments the oligonucleotide, or a contiguous nucleotide sequence of at least 12 nucleotides thereof, is at least 90% complementary, such as fully (or 100%) complementary to a region of a sequence selected from the group consisting of SEQ ID NOs 124-230.

In some embodiments the oligonucleotide, or a contiguous nucleotide sequence of at least 12 nucleotides thereof, is at least 90% complementary, such as fully (or 100%) complementary to a region of SEQ ID NO 186.

In some embodiments the oligonucleotide, or a contiguous nucleotide sequence of at least 12 nucleotides thereof, is at least 90% complementary, such as fully (or 100%) complementary to a region of SEQ ID NO 192.

In some embodiments the oligonucleotide, or a contiguous nucleotide sequence of at least 12 nucleotides thereof, is at least 90% complementary, such as fully (or 100%) complementary to a region of SEQ ID NO 205.

In some embodiments the oligonucleotide, or a contiguous nucleotide sequence of at least 13 nucleotides thereof, is at least 90% complementary, such as fully (or 100%) complementary to a region of SEQ ID NO 186.

In some embodiments the oligonucleotide, or a contiguous nucleotide sequence of at least 13 nucleotides thereof, is at least 90% complementary, such as fully (or 100%) complementary to a region of SEQ ID NO 192.

In some embodiments the oligonucleotide, or a contiguous nucleotide sequence of at least 13 nucleotides thereof, is at least 90% complementary, such as fully (or 100%) complementary to a region of SEQ ID NO 205.

In some embodiments the oligonucleotide, or a contiguous nucleotide sequence of at least 14 nucleotides thereof, is fully (or 100%) complementary to a sequence selected from the group consisting of SEQ ID NO 113, 114, 115, 116, 117 and 231.

In some embodiments the oligonucleotide, or a contiguous nucleotide sequence of at least 14 nucleotides thereof, is at least 90% complementary, such as fully (or 100%) complementary to a region of SEQ ID NO 186.

In some embodiments the oligonucleotide, or a contiguous nucleotide sequence of at least 14 nucleotides thereof, is at least 90% complementary, such as fully (or 100%) complementary to a region of SEQ ID NO 192.

In some embodiments the oligonucleotide, or a contiguous nucleotide sequence of at least 14 nucleotides thereof, is at least 90% complementary, such as fully (or 100%) complementary to a region of SEQ ID NO 205.

In some embodiments the oligonucleotide, or a contiguous nucleotide sequence of at least 15 nucleotides thereof, is at least 90% complementary, such as fully (or 100%) complementary to a region of SEQ ID NO 186.

In some embodiments the oligonucleotide, or a contiguous nucleotide sequence of at least 15 nucleotides thereof, is at least 90% complementary, such as fully (or 100%) complementary to a region of SEQ ID NO 192.

In some embodiments the oligonucleotide, or a contiguous nucleotide sequence of at least 15 nucleotides thereof, is at least 90% complementary, such as fully (or 100%) complementary to a region of SEQ ID NO 205.

In some embodiments the oligonucleotide, or a contiguous nucleotide sequence of at least 16 nucleotides thereof, is fully (or 100%) complementary to a sequence selected from the group consisting of SEQ ID NO SEQ ID NO 113, 114, 115, 116, 117 and 231.

In some embodiments the oligonucleotide, or a contiguous nucleotide sequence of at least 16 nucleotides thereof, is at least 90% complementary, such as fully (or 100%) complementary to a region of SEQ ID NO 186.

In some embodiments the oligonucleotide, or a contiguous nucleotide sequence of at least 16, such as 16, 17 or 18 nucleotides thereof, is at least 90% complementary, such as fully (or 100%) complementary to a region of SEQ ID NO 192.

In some embodiments the oligonucleotide, or a contiguous nucleotide sequence of at least 16 nucleotides thereof, is at least 90% complementary, such as fully (or 100%) complementary to a region of SEQ ID NO 205.

In some embodiments the oligonucleotide, or contiguous nucleotide region thereof is fully (or 100%) complementary to a sequence selected from the group consisting of a sequence selected from the group consisting of SEQ ID NO SEQ ID NO 113, 114, 115, 116, 117 and 231.

In some embodiments the oligonucleotide, or contiguous nucleotide region thereof is fully (or 100%) complementary to a sequence selected from the group consisting of a sequence selected from the group consisting of SEQ ID NO 124-230.

In some embodiments the oligonucleotide, or contiguous nucleotide region thereof is fully (or 100%) complementary to SEQ ID NO 186.

In some embodiments the oligonucleotide, or contiguous nucleotide region thereof is fully (or 100%) complementary to SEQ ID NO 192.

In some embodiments the oligonucleotide, or contiguous nucleotide region thereof is fully (or 100%) complementary to SEQ ID NO 205.

It is understood that the oligonucleotide motif sequences can be modified to for example increase nuclease resistance and/or binding affinity to the target nucleic acid. Modifications are described in the definitions and in the “Oligonucleotide design” section.

In some embodiments, the oligonucleotide of the invention, or contiguous nucleotide region thereof is fully complementary (100% complementary) to a region of the target nucleic acid, or in some embodiments may comprise one or two mismatches between the oligonucleotide and the target nucleic acid. In some embodiments the oligonucleotide, or contiguous nucleotide sequence of at least 12 nucleotides thereof, is at least 90% complementary, such as fully (or 100%) complementary to the target nucleic acid sequence.

In some embodiments the oligonucleotide, or a contiguous nucleotide sequence of at least 12 nucleotides thereof, has 100% identity to a sequence selected from the group consisting of SEQ ID NOs 5-111.

In some embodiments the oligonucleotide, or a contiguous nucleotide sequence of at least 14 nucleotides thereof, has 100% identity to a sequence selected from the group consisting of SEQ ID NOs 5-111

In some embodiments the oligonucleotide, or contiguous nucleotide sequence of at least 16 nucleotides thereof, has 100% identity to a sequence selected from the group consisting of SEQ ID NOs 5-111

In some embodiments the oligonucleotide, or contiguous nucleotide region thereof, comprises or consists of a sequence selected from SEQ ID NOs 5-111.

In some embodiments the oligonucleotide of the invention is selected from the following group (Note the target subsequence is the reverse complement of the oligonucleotide motif):

| | | | Target subsequence | |
|---|---|---|---|---|
| SEQ ID NO | Motif | Compound Design | SEQ ID | Target subsequence |
| 5 | agttaaaggaggagacaaat | AGTTaaaggaggagacAAAT | 124 | atttgtctcctcctttaact |
| 6 | tcagttaaaggaggagacaa | TCAgttaaaggaggagaCAA | 125 | ttgtctcctcctttaactga |
| 7 | ctcagttaaaggaggagaca | CTCagttaaaggaggagaCA | 126 | tgtctcctcctttaactgag |
| 8 | ctcagttaaaggaggagac | CTCagttaaaggaggaGAC | 127 | gtctcctcctttaactgag |
| 9 | actcagttaaaggaggagac | ACTCagttaaaggaggagAC | 128 | gtctcctcctttaactgagt |
| 10 | actcagttaaaggaggaga | ACTCagttaaaggaggaGA | 129 | tctcctcctttaactgagt |
| 11 | actcagttaaaggaggag | ACtcagttaaaggaGGAG | 130 | ctcctcctttaactgagt |
| 12 | gatgactcagttaaaggagg | GAtgactcagttaaaggAGG | 131 | cctcctttaactgagtcatc |
| 13 | atgatgactcagttaaagga | ATGAtgactcagttaaagGA | 132 | tcctttaactgagtcatcat |
| 14 | tgatgactcagttaaagg | TGAtgactcagttaAAGG | 133 | cctttaactgagtcatca |
| 15 | gatgatgactcagttaaagg | GAtgatgactcagttaAAGG | 134 | cctttaactgagtcatcatc |
| 16 | gatgatgactcagttaaag | GATGatgactcagttaAAG | 135 | ctttaactgagtcatcatc |
| 17 | tatcgactgcattagttgg | TATcgactgcattagttGG | 136 | ccaactaatgcagtcgata |
| 18 | gtatcgactgcattagttgg | GtatcgactgcattagttGG | 137 | ccaactaatgcagtcgatac |
| 19 | tcgactgcattagttg | TCGactgcattagTTG | 138 | caactaatgcagtcga |
| 19 | tcgactgcattagttg | TCGactgcattagtTG | 138 | caactaatgcagtcga |
| 19 | tcgactgcattagttg | TCGActgcattaGTTG | 138 | caactaatgcagtcga |
| 20 | tatcgactgcattagttg | TAtcgactgcattaGTTG | 139 | caactaatgcagtcgata |
| 21 | gtatcgactgcattagttg | GTAtcgactgcattagtTG | 140 | caactaatgcagtcgatac |
| 22 | tgtatcgactgcattagttg | TGtatcgactgcattagtTG | 141 | caactaatgcagtcgataca |
| 23 | atcgactgcattagtt | ATCgactgcattaGTT | 142 | aactaatgcagtcgat |
| 23 | atcgactgcattagtt | ATCGactgcattAGTT | 142 | aactaatgcagtcgat |
| 23 | atcgactgcattagtt | ATCGactgcattaGTT | 142 | aactaatgcagtcgat |
| 24 | tatcgactgcattagtt | TATCgactgcattaGTT | 143 | aactaatgcagtcgata |
| 25 | gtatcgactgcattagtt | GTATcgactgcattagTT | 144 | aactaatgcagtcgatac |
| 26 | tgtatcgactgcattagtt | TGTatcgactgcattagTT | 145 | aactaatgcagtcgataca |
| 27 | ttgtatcgactgcattagtt | TTGtatcgactgcattagTT | 146 | aactaatgcagtcgatacaa |
| 28 | tatcgactgcattagt | TATcgactgcattaGT | 147 | actaatgcagtcgata |
| 28 | tatcgactgcattagt | TATCgactgcatTAGT | 147 | actaatgcagtcgata |
| 29 | gtatcgactgcattagt | GTATcgactgcattaGT | 148 | actaatgcagtcgatac |
| 30 | tgtatcgactgcattagt | TGTatcgactgcattaGT | 149 | actaatgcagtcgataca |
| 31 | gtatcgactgcattag | GTAtcgactgcatTAG | 150 | ctaatgcagtcgatac |
| 31 | gtatcgactgcattag | GTAtcgactgcattAG | 150 | ctaatgcagtcgatac |
| 31 | gtatcgactgcattag | GTATcgactgcaTTAG | 150 | ctaatgcagtcgatac |
| 32 | tgtatcgactgcattag | TGtatcgactgcaTTAG | 151 | ctaatgcagtcgataca |
| 33 | ttgtatcgactgcattag | TTGtatcgactgcatTAG | 152 | ctaatgcagtcgatacaa |
| 34 | attgtatcgactgcattag | ATtgtatcgactgcaTTAG | 153 | ctaatgcagtcgatacaat |
| 35 | tgtatcgactgcatta | TGTatcgactgcaTTA | 154 | taatgcagtcgataca |
| 35 | tgtatcgactgcatta | TGTAtcgactgcATTA | 154 | taatgcagtcgataca |
| 36 | attgtatcgactgcatta | ATTGtatcgactgcaTTA | 155 | taatgcagtcgatacaat |
| 37 | ttgtatcgactgcatt | TTGtatcgactgcaTT | 156 | aatgcagtcgatacaa |
| 37 | ttgtatcgactgcatt | TTGtatcgactgCATT | 156 | aatgcagtcgatacaa |
| 38 | attgtatcgactgcat | ATTgtatcgactgCAT | 157 | atgcagtcgatacaat |
| 38 | attgtatcgactgcat | ATTgtatcgactgcAT | 157 | atgcagtcgatacaat |
| 38 | attgtatcgactgcat | ATTGtatcgactGCAT | 157 | atgcagtcgatacaat |
| 39 | acgcattgtatcgact | ACGcattgtatcgACT | 158 | agtcgatacaatgcgt |
| 39 | acgcattgtatcgact | ACGCattgtatcGACT | 158 | agtcgatacaatgcgt |
| 40 | tacgcattgtatcgac | TACgcattgtatcGAC | 159 | gtcgatacaatgcgta |
| 40 | tacgcattgtatcgac | TACGcattgtatCGAC | 159 | gtcgatacaatgcgta |
| 41 | ctacgcattgtatcgac | CTacgcattgtatCGAC | 160 | gtcgatacaatgcgtag |
| 42 | tctacgcattgtatcgac | TCTAcgcattgtatcgAC | 161 | gtcgatacaatgcgtaga |
| 43 | atctacgcattgtatcgac | ATCtacgcattgtatcgAC | 162 | gtcgatacaatgcgtagat |
| 44 | tatctacgcattgtatcgac | TAtctacgcattgtatcGAC | 163 | gtcgatacaatgcgtagata |
| 45 | ctacgcattgtatcga | CTAcgcattgtatCGA | 164 | tcgatacaatgcgtag |
| 45 | ctacgcattgtatcga | CTACgcattgtaTCGA | 164 | tcgatacaatgcgtag |
| 46 | tatctacgcattgtatcga | TAtctacgcattgtatCGA | 165 | tcgatacaatgcgtagata |
| 47 | tctacgcattgtatcg | TCTacgcattgtaTCG | 166 | cgatacaatgcgtaga |
| 47 | tctacgcattgtatcg | TCTacgcattgtatCG | 166 | cgatacaatgcgtaga |
| 47 | tctacgcattgtatcg | TCTAcgcattgtATCG | 166 | cgatacaatgcgtaga |
| 48 | atctacgcattgtatcg | ATCTacgcattgtaTCG | 167 | cgatacaatgcgtagat |
| 49 | tatctacgcattgtatcg | TATCtacgcattgtatCG | 168 | cgatacaatgcgtagata |
| 50 | tctatctacgcattgtatcg | TCtatctacgcattgtatCG | 169 | cgatacaatgcgtagataga |
| 51 | atctacgcattgtatc | ATCtacgcattgtATC | 170 | gatacaatgcgtagat |
| 51 | atctacgcattgtatc | ATCTacgcattgTATC | 170 | gatacaatgcgtagat |
| 52 | tatctacgcattgtatc | TATctacgcattgTATC | 171 | gatacaatgcgtagata |
| 53 | ctatctacgcattgtatc | CTatctacgcattgTATC | 172 | gatacaatgcgtagatag |
| 54 | tctatctacgcattgtatc | TCTatctacgcattgtaTC | 173 | gatacaatgcgtagataga |
| 55 | ttctatctacgcattgtatc | TTCtatctacgcattgtaTC | 174 | gatacaatgcgtagatagaa |
| 56 | tatctacgcattgtat | TATctacgcattgTAT | 175 | atacaatgcgtagata |
| 56 | tatctacgcattgtat | TATCtacgcattGTAT | 175 | atacaatgcgtagata |
| 57 | ctatctacgcattgtat | CTAtctacgcattGTAT | 176 | atacaatgcgtagatag |
| 58 | tctatctacgcattgtat | TCtatctacgcattGTAT | 177 | atacaatgcgtagataga |
| 59 | ttctatctacgcattgtat | TTCtatctacgcattgTAT | 178 | atacaatgcgtagatagaa |
| 60 | ctatctacgcattgta | CTAtctacgcattGTA | 179 | tacaatgcgtagatag |
| 60 | ctatctacgcattgta | CTATctacgcatTGTA | 179 | tacaatgcgtagatag |
| 61 | tctatctacgcattgta | TCTatctacgcattGTA | 180 | tacaatgcgtagataga |
| 62 | ttctatctacgcattgta | TTCtatctacgcattGTA | 181 | tacaatgcgtagatagaa |
| 63 | ttctatctacgcattgt | TTCtatctacgcatTGT | 182 | acaatgcgtagatagaa |
| 64 | tcttctatctacgcattgt | TCttctatctacgcattGT | 183 | acaatgcgtagatagaaga |
| 65 | ttcttctatctacgcattgt | TtcttctatctacgcattGT | 184 | acaatgcgtagatagaagaa |
| 66 | ttcttctatctacgcattg | TTCttctatctacgcatTG | 185 | caatgcgtagatagaagaa |
| 67 | ttctatctacgcattg | TTCtatctacgcaTTG | 186 | caatgcgtagatagaa |
| 68 | cttctatctacgcatt | CTTCtatctacgCATT | 187 | aatgcgtagatagaag |
| 69 | tcttctatctacgcatt | TCTtctatctacgCATT | 188 | aatgcgtagatagaaga |
| 70 | ttcttctatctacgcatt | TTCTtctatctacgcATT | 189 | aatgcgtagatagaagaa |
| 71 | tcttctatctacgcat | TCTTctatctacgCAT | 190 | atgcgtagatagaaga |
| 72 | ttcttctatctacgcat | TTCTtctatctacgCAT | 191 | atgcgtagatagaagaa |
| 73 | cttcttctatctacgcat | CTTCttctatctacgcAT | 192 | atgcgtagatagaagaag |
| 74 | ttcttctatctacgca | TTCttctatctacGCA | 193 | tgcgtagatagaagaa |
| 75 | cttcttctatctacgca | CTTCttctatctacgCA | 194 | tgcgtagatagaagaag |
| 76 | gcttcttctatctacgca | GcttcttctatctacgCA | 195 | tgcgtagatagaagaagc |
| 77 | cttcttctatctacgc | CTtcttctatctACGC | 196 | gcgtagatagaagaag |
| 78 | gcttcttctatctacg | GCTtcttctatctACG | 197 | cgtagatagaagaagc |
| 79 | cgtggggcttcttcta | CGTggggcttcttCTA | 198 | tagaagaagccccacg |
| 80 | tgacttggagaaaagcacaa | TGacttggagaaaagcacAA | 199 | ttgtgcttttctccaagtca |
| 81 | ctgacttggagaaaagcac | CtgacttggagaaaagcAC | 200 | gtgcttttctccaagtcag |
| 82 | agagtcatcgtgctcc | AGAgtcatcgtgcTCC | 201 | ggagcacgatgactct |
| 83 | aagtactttaatagctcaaa | AAGTactttaatagctCAAA | 202 | tttgagctattaaagtactt |
| 84 | aagtactttaatagctcaa | AAGTactttaatagcTCAA | 203 | ttgagctattaaagtactt |
| 85 | gaagtactttaatagctcaa | GAAGtactttaatagctCAA | 204 | ttgagctattaaagtacttc |
| 86 | tactttaatagctcaa | TACTttaatagcTCAA | 205 | ttgagctattaaagta |
| 87 | aagtactttaatagctca | AAGTactttaatagcTCA | 206 | tgagctattaaagtactt |
| 88 | gaagtactttaatagctca | GAAGtactttaatagcTCA | 207 | tgagctattaaagtacttc |
| 89 | agaagtactttaatagctc | AGAAgtactttaatagCTC | 208 | gagctattaaagtacttct |
| 90 | aagaagtactttaatagctc | AAGAagtactttaatagCTC | 209 | gagctattaaagtacttctt |
| 91 | gaagtactttaatagct | GAAGtactttaatAGCT | 210 | agctattaaagtacttc |
| 92 | taagaagtactttaatagct | TAAgaagtactttaatAGCT | 211 | agctattaaagtacttctta |
| 93 | agaagtactttaatagc | AGAAgtactttaaTAGC | 212 | gctattaaagtacttct |
| 94 | taagaagtactttaatagc | TAAGaagtactttaaTAGC | 213 | gctattaaagtacttctta |
| 95 | gtaagaagtactttaatagc | GTaagaagtactttaaTAGC | 214 | gctattaaagtacttcttac |
| 96 | taagaagtactttaatag | TAAGaagtactttaATAG | 215 | ctattaaagtacttctta |
| 97 | gtaagaagtactttaatag | GTAAgaagtactttaATAG | 216 | ctattaaagtacttcttac |
| 98 | tgtaagaagtactttaatag | TGTAagaagtactttaATAG | 217 | ctattaaagtacttcttaca |
| 99 | aatgtgtaagaagtacttt | AATGtgtaagaagtaCTTT | 218 | aaagtacttcttacacatt |
| 100 | caatgtgtaagaagtacttt | CAATgtgtaagaagtaCTTT | 219 | aaagtacttcttacacattg |
| 101 | atgtgtaagaagtactt | ATGTgtaagaagtACTT | 220 | aagtacttcttacacat |
| 102 | aatgtgtaagaagtactt | AATGtgtaagaagtACTT | 221 | aagtacttcttacacatt |
| 103 | caatgtgtaagaagtactt | CAATgtgtaagaagtACTT | 222 | aagtacttcttacacattg |
| 104 | gcaatgtgtaagaagtactt | GCaatgtgtaagaagtACTT | 223 | aagtacttcttacacattgc |
| 105 | atgtgtaagaagtact | ATGtgtaagaagtACT | 224 | agtacttcttacacat |
| 105 | atgtgtaagaagtact | ATGTgtaagaagTACT | 224 | agtacttcttacacat |
| 106 | gcaatgtgtaagaagtact | GCAAtgtgtaagaagtACT | 225 | agtacttcttacacattgc |
| 107 | aatgtgtaagaagtac | AATGtgtaagaaGTAC | 226 | gtacttcttacacatt |
| 107 | aatgtgtaagaagtac | AATgtgtaagaaGTAC | 226 | gtacttcttacacatt |
| 108 | caatgtgtaagaagtac | CAATgtgtaagaaGTAC | 227 | gtacttcttacacattg |
| 109 | gcaatgtgtaagaagtac | GCAatgtgtaagaaGTAC | 228 | gtacttcttacacattgc |
| 110 | caatgtgtaagaagta | CAAtgtgtaagaaGTA | 229 | tacttcttacacattg |
| 110 | caatgtgtaagaagta | CAAtgtgtaagaAGTA | 229 | tacttcttacacattg |
| 110 | caatgtgtaagaagta | CAATgtgtaagaAGTA | 229 | tacttcttacacattg |
| 111 | gcaatgtgtaagaagta | GCAatgtgtaagaAGTA | 230 | tacttcttacacattgc |

or conjugate thereof; wherein for the column entitled compound design, capital letters are LNA nucleosides, lower case letters are DNA nucleosides, cytosine nucleosides are optionally 5 methyl cytosine, and internucleoside linkages are at least 80%, such as at least 90% or 100% modified internucleoside linkages, such as phosphorothioate internucleoside linkages. In some embodiments all internucleoside linkages of the compounds in the compound design column in the above table are phosphorothioate internucleoside linkages. The motif and target subsequence sequences are nucleobase sequences.

The invention provides the following oligonucleotides:

| CMP ID NO | Compound |
|---|---|
| 5.1 | AGTTaaaggaggagacAAAT |
| 6.1 | TCAgttaaaggaggagaCAA |
| 7.1 | CTCagttaaaggaggagaCA |
| 8.1 | CTCagttaaaggaggaGAC |
| 9.1 | ACTCagttaaaggaggagAC |
| 10.1 | ACTCagttaaaggaggaGA |
| 11.1 | ACtcagttaaaggaGGAG |
| 12.1 | GAtgactcagttaaaggAGG |
| 13.1 | ATGAtgactcagttaaagGA |
| 14.1 | TGAtgactcagttaAAGG |
| 15.1 | GAtgatgactcagttaAAGG |
| 16.1 | GATGatgactcagttaAAG |
| 17.1 | TAT^{m}cgactgcattagttGG |
| 18.1 | Gtat^{m}cgactgcattagttGG |
| 19.1 | TCGactgcattagTTG |
| 19.2 | TCGactgcattagtTG |
| 19.3 | TCGActgcattaGTTG |
| 20.1 | TAt^{m}cgactgcattaGTTG |
| 21.1 | GTAt^{m}cgactgcattagtTG |
| 22.1 | TGtat^{m}cgactgcattagtTG |
| 23.1 | ATCgactgcattaGTT |
| 23.2 | ATCGactgcattAGTT |
| 23.3 | ATCGactgcattaGTT |
| 24.1 | TATCgactgcattaGTT |
| 25.1 | GTAT^{m}cgactgcattagTT |
| 26.1 | TGTat^{m}cgactgcattagTT |
| 27.1 | TTGtat^{m}cgactgcattagTT |
| 28.1 | TAT^{m}cgactgcattaGT |
| 28.2 | TATCgactgcatTAGT |
| 29.1 | GTAT^{m}cgactgcattaGT |
| 30.1 | TGTat^{m}cgactgcattaGT |
| 31.1 | GTAt^{m}cgactgcatTAG |
| 31.2 | GTAt^{m}cgactgcattAG |
| 31.3 | GTAT^{m}cgactgcaTTAG |
| 32.1 | TGtat^{m}cgactgcaTTAG |
| 33.1 | TTGtat^{m}cgactgcatTAG |
| 34.1 | ATtgtat^{m}cgactgcaTTAG |
| 35.1 | TGTat^{m}cgactgcaTTA |
| 35.2 | TGTAt^{m}cgactgcATTA |
| 36.1 | ATTGtat^{m}cgactgcaTTA |
| 37.1 | TTGtat^{m}cgactgcaTT |
| 37.2 | TTGtat^{m}cgactgCATT |
| 38.1 | ATTgtat^{m}cgactgCAT |
| 38.2 | ATTgtat^{m}cgactgcAT |
| 38.3 | ATTGtat^{m}cgactGCAT |
| 39.1 | ACGcattgtat^{m}cgACT |
| 39.2 | ACGCattgtat^{m}cGACT |
| 40.1 | TACgcattgtat^{m}cGAC |
| 40.2 | TACGcattgtatCGAC |
| 41.1 | CTa^{m}cgcattgtatCGAC |
| 42.1 | TCTA^{m}cgcattgtat^{m}cgAC |
| 43.1 | ATCta^{m}cgcattgtat^{m}cgAC |
| 44.1 | TAtcta^{m}cgcattgtatcGAC |
| 45.1 | CTA^{m}cgcattgtatCGA |
| 45.2 | CTACgcattgtaTCGA |
| 46.1 | TAtcta^{m}cgcattgtatCGA |
| 47.1 | TCTa^{m}cgcattgtaTCG |
| 47.2 | TCTa^{m}cgcattgtatCG |
| 47.3 | TCTA^{m}cgcattgtATCG |
| 48.1 | ATCTa^{m}cgcattgtaTCG |
| 49.1 | TATCta^{m}cgcattgtatCG |
| 50.1 | TCtatcta^{m}cgcattgtatCG |
| 51.1 | ATCta^{m}cgcattgtATC |
| 51.2 | ATCTa^{m}cgcattgTATC |
| 52.1 | TATcta^{m}cgcattgTATC |
| 53.1 | CTatcta^{m}cgcattgTATC |
| 54.1 | TCTatcta^{m}cgcattgtaTC |
| 55.1 | TTCtatcta^{m}cgcattgtaTC |
| 56.1 | TATcta^{m}cgcattgTAT |
| 56.2 | TATCta^{m}cgcattGTAT |
| 57.1 | CTAtcta^{m}cgcattGTAT |
| 58.1 | TCtatcta^{m}cgcattGTAT |
| 59.1 | TTCtatcta^{m}cgcattgTAT |
| 60.1 | CTAtcta^{m}cgcattGTA |
| 60.2 | CTATcta^{m}cgcatTGTA |
| 61.1 | TCTatcta^{m}cgcattGTA |
| 62.1 | TTCtatcta^{m}cgcattGTA |
| 63.1 | TTCtatcta^{m}cgcatTGT |
| 64.1 | TCttctatcta^{m}cgcattGT |
| 65.1 | Ttcttctatcta^{m}cgcattGT |
| 66.1 | TTCttctatcta^{m}cgcatTG |
| 67.1 | TTCtatcta^{m}cgcaTTG |
| 68.1 | CTTCtatcta^{m}cgCATT |
| 69.1 | TCTtctatcta^{m}cgCATT |
| 70.1 | TTCTtctatcta^{m}cgcATT |
| 71.1 | TCTTctatcta^{m}cgCAT |
| 72.1 | TTCTtctatcta^{m}cgCAT |
| 73.1 | CTTCttctatcta^{m}cgcAT |
| 74.1 | TTCttctatctacGCA |
| 75.1 | CTTCttctatcta^{m}cgCA |
| 76.1 | Gcttcttctatcta^{m}cgCA |
| 77.1 | CTtcttctatctACGC |
| 78.1 | GCTtcttctatctACG |
| 79.1 | CGTggggcttcttCTA |
| 80.1 | TGacttggagaaaagcacAA |
| 81.1 | CtgacttggagaaaagcAC |
| 82.1 | AGAgtcat^{m}cgtgcTCC |
| 83.1 | AAGTactttaatagctCAAA |
| 84.1 | AAGTactttaatagcTCAA |
| 85.1 | GAAGtactttaatagctCAA |
| 86.1 | TACTttaatagcTCAA |
| 87.1 | AAGTactttaatagcTCA |
| 88.1 | GAAGtactttaatagcTCA |
| 89.1 | AGAAgtactttaatagCTC |
| 90.1 | AAGAagtactttaatagCTC |
| 91.1 | GAAGtactttaatAGCT |
| 92.1 | TAAgaagtactttaatAGCT |
| 93.1 | AGAAgtactttaaTAGC |
| 94.1 | TAAGaagtactttaaTAGC |
| 95.1 | GTaagaagtactttaaTAGC |
| 96.1 | TAAGaagtactttaATAG |
| 97.1 | GTAAgaagtactttaATAG |
| 98.1 | TGTAagaagtactttaATAG |
| 99.1 | AATGtgtaagaagtaCTTT |
| 100.1 | CAATgtgtaagaagtaCTTT |
| 101.1 | ATGTgtaagaagtACTT |
| 102.1 | AATGtgtaagaagtACTT |
| 103.1 | CAATgtgtaagaagtACTT |
| 104.1 | GCaatgtgtaagaagtACTT |
| 105.1 | ATGtgtaagaagtACT |
| 105.2 | ATGTgtaagaagTACT |
| 106.1 | GCAAtgtgtaagaagtACT |
| 107.1 | AATGtgtaagaaGTAC |
| 107.2 | AATgtgtaagaaGTAC |
| 108.1 | CAATgtgtaagaaGTAC |
| 109.1 | GCAatgtgtaagaaGTAC |
| 110.1 | CAAtgtgtaagaaGTA |
| 110.2 | CAAtgtgtaagaAGTA |
| 110.3 | CAATgtgtaagaAGTA |
| 111.1 | GCAatgtgtaagaAGTA |

or a conjugate thereof; wherein in the compounds of the above table, capital letters represent beta-D-oxy LNA nucleosides, all LNA cytosines are 5-methyl cytosine (as indicated by the superscript m), lower case letters represent DNA nucleosides, superscript m before a lower case c represents a 5 methyl cytosine DNA nucleoside. All internucleoside linkages are phosphorothioate internucleoside linkages.

### Oligonucleotide Design

Oligonucleotide design refers to the pattern of nucleoside sugar modifications in the oligonucleotide sequence. The oligonucleotides of the invention comprise sugar-modified nucleosides and may also comprise DNA or RNA nucleosides. In some embodiments, the oligonucleotide comprises sugar-modified nucleosides and DNA nucleosides. Incorporation of modified nucleosides into the oligonucleotide of the invention may enhance the affinity of the oligonucleotide for the target nucleic acid. In that case, the modified nucleosides can be referred to as affinity enhancing modified nucleotides.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. In an embodiment, the oligonucleotide of the invention may comprise modifications, which are independently selected from these three types of modifications (modified sugar, modified nucleobase and modified internucleoside linkage) or a combination thereof. Preferably the oligonucleotide comprises one or more sugar modified nucleosides, such as 2′ sugar modified nucleosides. Preferably the oligonucleotide of the invention comprise the one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. Even more preferably the one or more modified nucleoside is LNA.

In some embodiments, at least 1 of the modified nucleosides is a locked nucleic acid (LNA), such as at least 2, such as at least 3, at least 4, at least 5, at least 6, at least 7, or at least 8 of the modified nucleosides are LNA. In a still further embodiment all the modified nucleosides are LNA.

In a further embodiment the oligonucleotide comprises at least one modified internucleoside linkage. In a preferred embodiment the the internucleoside linkages within the contiguous nucleotide sequence are phosphorothioate or boranophosphate internucleoside linkages. In some embodiments all the internucleotide linkages in the contiguous sequence of the oligonucleotide are phosphorothioate linkages.

In some embodiments, the oligonucleotide of the invention comprise at least one modified nucleoside which is a 2′-MOE-RNA, such as 2, 3, 4, 5, 6, 7, 8, 9 or 10 2′-MOE-RNA nucleoside units. In some embodiments, at least one of said modified nucleoside is 2′-fluoro DNA, such as 2, 3, 4, 5, 6, 7, 8, 9 or 10 2′-fluoro-DNA nucleoside units.

In some embodiments, the oligonucleotide of the invention comprises at least one LNA unit, such as 1, 2, 3, 4, 5, 6, 7, or 8 LNA units, such as from 2 to 6 LNA units, such as from 3 to 7 LNA units, 4 to 8 LNA units or 3, 4, 5, 6 or 7 LNA units. In some embodiments, all the modified nucleosides are LNA nucleosides. In some embodiments, all LNA cytosine units are 5-methyl-cytosine. In some embodiments the oligonucleotide or contiguous nucleotide region thereof has at least 1 LNA unit at the 5′ end and at least 2 LNA units at the 3′ end of the nucleotide sequence. In some embodiments all cytosine nucleobases present in the oligonucleotide of the invention are 5-methyl-cytosine.

In some embodiments, the oligonucleotide of the invention comprises at least one LNA unit and at least one 2′ substituted modified nucleoside.

In some embodiments of the invention, the oligonucleotide comprise both 2′ sugar modified nucleosides and DNA units.

In an embodiment of the invention the oligonucleotide of the invention is capable of recruiting RNase H.

In some embodiments, the oligonucleotide of the invention or contiguous nucleotide region thereof is a gapmers oligonucleotide.

### Gapmer Design

In some embodiments the oligonucleotide of the invention, or contiguous nucleotide region thereof, has a gapmer design or structure also referred herein merely as “Gapmer”. In a gapmer structure the oligonucleotide comprises at least three distinct structural regions a 5′-flank, a gap and a 3′-flank, F-G-F′ in ‘5->3’ orientation. In this design, flanking regions F and F′ (also termed wing regions) comprise at least one sugar modified nucleoside which is adjacent to region G, and may in some embodiments comprise a contiguous stretch of 2-7 sugar modified nucleoside, or a contiguous stretch of sugar modified and DNA nucleosides (mixed wings comprising both sugar modified and DNA nucleosides). Consequently, the nucleosides of the 5′ flanking region and the 3′ flanking region which are adjacent to the gap region are sugar modified nucleosides, such as 2′ modified nucleosides. The gap region, G, comprises a contiguous stretch of nucleotides which are capable of recruiting RNase H, when the oligonucleotide is in duplex with the HTRA1 target nucleic acid. In some embodiments, region G comprises a contiguous stretch of 5-16 DNA nucleosides. The gapmer region F-G-F′ is complementary to the HTRA1 target nucleic acid, and may therefore be the contiguous nucleotide region of the oligonucleotide.

Regions F and F′, flanking the 5′ and 3′ ends of region G, may comprise one or more affinity enhancing modified nucleosides. In some embodiments, the 3′ flank comprises at least one LNA nucleoside, preferably at least 2 LNA nucleosides. In some embodiments, the 5′ flank comprises at least one LNA nucleoside. In some embodiments both the 5′ and 3′ flanking regions comprise a LNA nucleoside. In some embodiments all the nucleosides in the flanking regions are LNA nucleosides. In other embodiments, the flanking regions may comprise both LNA nucleosides and other nucleosides (mixed flanks), such as DNA nucleosides and/or non-LNA modified nucleosides, such as 2′ substituted nucleosides. In this case the gap is defined as a contiguous sequence of at least 5 RNase H recruiting nucleosides (such as 5-16 DNA nucleosides) flanked at the 5′ and 3′ end by an affinity enhancing modified nucleoside, such as an LNA, such as beta-D-oxy-LNA.

### Region F

Region F (5′ flank or 5′ wing) attached to the '5 end of region G comprises, contains or consists of at least one sugar modified nucleoside such as at least 2, at least 3, at least 4, at least 5, at least 6, at least 7 modified nucleosides. In some embodiments region F comprises or consists of from 1 to 7 modified nucleosides, such as from 2 to 6 modified nucleosides, such as from 2 to 5 modified nucleosides, such as from 2 to 4 modified nucleosides, such as from 1 to 3 modified nucleosides, such as 1, 2, 3 or 4 modified nucleosides.

In an embodiment, one or more or all of the modified nucleosides in region F are 2′ modified nucleosides.

In a further embodiment one or more of the 2′ modified nucleosides in region F are selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In one embodiment of the invention all the modified nucleosides in region F are LNA nucleosides. In a further embodiment the LNA nucleosides in region F are independently selected from the group consisting of oxy-LNA, thio-LNA, amino-LNA, cET, and/or ENA, in either the beta-D or alpha-L configurations or combinations thereof. In a preferred embodiment region F has at least 1 beta-D-oxy LNA unit, at the 5′ end of the contiguous sequence.

### Region G

Region G (gap region) may comprise, contain or consist of at 5-16 consecutive DNA nucleosides capable of recruiting RNaseH. In a further embodiment region G comprise, contain or consist of from 5 to 12, or from 6 to 10 or from 7 to 9, such as 8 consecutive nucleotide units capable of recruiting RNaseH.

In a still further embodiment at least one nucleoside unit in region G is a DNA nucleoside unit, such as from 4 to 20 or or 6 to 18 DNA units, such as 5 to 16, In some embodiments, all of the nucleosides of region G are DNA units.

In further embodiments the region G may consist of a mixture of DNA and other nucleosides capable of mediating RNase H cleavage. In some embodiments, at least 50% of the nucleosides of region G are DNA, such as at least 60%, at least 70% or at least 80%, or at least 90% DNA.

### Region F′

Region F′ (3′ flank or 3′ wing) attached to the ‘3 end of region G comprises, contains or consists of at least one sugar modified nucleoside such as at least 2, at least 3, at least 4, at least 5, at least 6, at least 7 modified nucleosides. In some embodiments region F’ comprises or consists of from 1 to 7 modified nucleosides, such as from 2 to 6 modified nucleosides, such as from 2 to 5 modified nucleosides, such as from 2 to 4 modified nucleosides, such as from 1 to 3 modified nucleosides, such as 1, 2, 3 or 4 modified nucleosides.

In an embodiment, one or more or all of the modified nucleosides in region F′ are 2′ modified nucleosides.

In a further embodiment one or more of the 2′ modified nucleosides in region F′ are selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In one embodiment of the invention all the modified nucleosides in region F′ are LNA nucleosides. In a further embodiment the LNA nucleosides in region F′ are independently selected from the group consisting of oxy-LNA, thio-LNA, amino-LNA, cET, and/or ENA, in either the beta-D or alpha-L configurations or combinations thereof. In a preferred embodiment region F′ has at least 1 beta-D-oxy LNA unit, at the 5′ end of the contiguous sequence.

### Region D, D′ and D″

The oligonucleotide of the invention comprises a contiguous nucleotide region which is complementary to the target nucleic acid. In some embodiments, the oligonucleotide may further comprise additional nucleotides positioned 5′ and/or 3′ to the contiguous nucleotide region, which are referred to as region D herein. Region D′ and D″ can be attached to the 5′ end of region F or the 3′ end of region F′, respectively. The D regions (region D′ or D″) may in some embodiments form part of the contiguous nucleotide sequence which is complementary to the target nucleic acid, or in other embodiments the D region(s) may be non-complementary to the target nucleic acid.

In some embodiments the oligonucleotide of the invention consists or comprises of the contiguous nucleotide region and optionally 1-5 additional 5′ nucleotides (region D′).

In some embodiments the oligonucleotide of the invention consists or comprises of the contiguous nucleotide region and optionally 1-5 additional 3′ nucleotides (region D″).

Region D′ or D″ may independently comprise 1, 2, 3, 4 or 5 additional nucleotides, which may be complementary or non-complementary to the target nucleic acid. In this respect the oligonucleotide of the invention, may in some embodiments comprise a contiguous nucleotide sequence capable of modulating the target which is flanked at the 5′ and/or 3′ end by additional nucleotides. Such additional nucleotides may serve as a nuclease susceptible biocleavable linker, and may therefore be used to attach a functional group such as a conjugate moiety to the oligonucleotide of the invention. In some embodiments the additional 5′ and/or 3′ end nucleotides are linked with phosphodiester linkages, and may be DNA or RNA. In another embodiment, the additional 5′ and/or 3′ end nucleotides are modified nucleotides which may for example be included to enhance nuclease stability or for ease of synthesis. In some embodiments the oligonucleotide of the invention comprises a region D′ and/or D″ in addition to the contiguous nucleotide region.

In some embodiments, the gapmer oligonucleotide of the present invention can be represented by the following formulae:

F-G-F′; in particular F₁₋₇-G₄₋₁₂-F′₁₋₇

D′-F-G-F′, in particular D′₁₋₃-F₁₋₇-G₄₋₁₂-F′₁₋₇

F-G-F′-D″, in particular F₁₋₇-G₄₋₁₂-F′₁₋₇-D″₁₋₃

D′-F-G-F′-D″, in particular D′₁₋₃-F₁₋₇-G₄₋₁₂-F′₁₋₇-D″₁₋₃

### Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand). In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

### Pharmaceutical Salts

For use as a therapeutic, the oligonucleotide of the invention may be provided as a suitable pharmaceutical salt, such as a sodium or potassium salt. In some embodiments the oligonucleotide of the invention is a sodium salt.

### Pharmaceutical Composition

In a further aspect, the invention provides pharmaceutical compositions comprising any of the aforementioned oligonucleotides and/or oligonucleotide conjugates and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. In some embodiments, the oligonucleotide of the invention is administered at a dose of 10-1000 μg.

WO 2007/031091 provides suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

### Applications

The oligonucleotides of the invention may be utilized as research reagents for, for example, diagnostics, therapeutics and prophylaxis.

In research, such oligonucleotides may be used to specifically modulate the synthesis of HTRA1 protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein.

In diagnostics the oligonucleotides may be used to detect and quantitate HTRA1 expression in cell and tissues by northern blotting, in-situ hybridisation or similar techniques.

For therapeutics, an animal or a human, suspected of having a disease or disorder, which can be treated by modulating the expression of HTRA1.

The invention provides methods for treating or preventing a disease, comprising administering a therapeutically or prophylactically effective amount of an oligonucleotide, an oligonucleotide conjugate or a pharmaceutical composition of the invention to a subject suffering from or susceptible to the disease.

The invention also relates to an oligonucleotide, a composition or a conjugate as defined herein for use as a medicament.

The oligonucleotide, oligonucleotide conjugate or a pharmaceutical composition according to the invention is typically administered in an effective amount.

The invention also provides for the use of the oligonucleotide or oligonucleotide conjugate of the invention as described for the manufacture of a medicament for the treatment of a disorder as referred to herein, or for a method of the treatment of as a disorder as referred to herein.

The disease or disorder, as referred to herein, is associated with expression of HTRA1. In some embodiments disease or disorder may be associated with a mutation in the HTRA1 gene or a gene whose protein product is associated with or interacts with HTRA1. Therefore, in some embodiments, the target nucleic acid is a mutated form of the HTRA1 sequence and in other embodiments, the target nucleic acid is a regulator of the HTRA1 sequence.

The methods of the invention are preferably employed for treatment or prophylaxis against diseases caused by abnormal levels and/or activity of HTRA1.

The invention further relates to use of an oligonucleotide, oligonucleotide conjugate or a pharmaceutical composition as defined herein for the manufacture of a medicament for the treatment of abnormal levels and/or activity of HTRA1.

In one embodiment, the invention relates to oligonucleotides, oligonucleotide conjugates or pharmaceutical compositions for use in the treatment of diseases or disorders selected from eye disorders, such as macular degeneration, including age related macular degeneration (AMD), such as dry AMD or wet AMD, and diabetic retinopathy. In some embodiments the oligonucleotide conjugates or pharmaceutical compositions of the invention may be for use in the treatment of geographic atrophy or intermediate dAMD. HTRA1 has also been indicated in Alzheimer's and Parkinson's disease, and therefore in some embodiments, the oligonucleotide conjugates or pharmaceutical compositions of the invention may be for use in the treatment of Alzheimer's or Parkinson's. HTRA1 has also been indicated in Duchenne muscular dystrophy, arthritis, such as osteoarthritis, familial ischemic cerebral small-vessel disease, and therefore in some embodiments, the oligonucleotide conjugates or pharmaceutical compositions of the invention may be for use in the treatment of Duchenne muscular dystrophy, arthritis, such as osteoarthritis, or familial ischemic cerebral small-vessel disease.

### Administration

The oligonucleotides or pharmaceutical compositions of the present invention may be administered topical (such as, to the skin, inhalation, ophthalmic or otic) or enteral (such as, orally or through the gastrointestinal tract) or parenteral (such as, intravenous, subcutaneous, intra-muscular, intracerebral, intracerebroventricular or intrathecal).

In some embodiments the oligonucleotide, conjugate or pharmaceutical compositions of the present invention are administered by a parenteral route including intravenous, intraarterial, subcutaneous, intraperitoneal or intramuscular injection or infusion, intrathecal or intracranial, e.g. intracerebral or intraventricular, administration. In some embodiments the active oligonucleotide or oligonucleotide conjugate is administered intravenously. In another embodiment the active oligonucleotide or oligonucleotide conjugate is administered subcutaneously.

For use in treating eye disorders, such as macular degeneration, e.g. AMD (wet or dry), intraocular injection may be used.

In some embodiments, the compound of the invention, or pharmaceutically acceptable salt thereof, is administered via an intraocular injection in a dose from about 10 μg to about 200 μg per eye, such as about 50 μg to about 150 μg per eye, such as about 100 μg per eye. In some embodiments, the dosage interval, i.e. the period of time between consecutive dosings is at least monthly, such as at least bi monthly or at least once every three months.

### Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above

### EXAMPLES

### Materials and Methods

### Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

### Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

### Purification by RP-HPLC:

The crude compounds are purified by preparative RP-HPLC on a Phenomenex Jupiter C18 10p 150×10 mm column. 0.1 M ammonium acetate pH 8 and acetonitrile is used as buffers at a flow rate of 5 mL/min. The collected fractions are lyophilized to give the purified compound typically as a white solid.

### Abbreviations

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
Ibu: Isobutyryl
RP-HPLC: Reverse phase high performance liquid chromatography

### Tₘ Assay:

Oligonucleotide and RNA target (phosphate linked, PO) duplexes are diluted to 3 mM in 500 ml RNase-free water and mixed with 500 ml 2×Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

### Oligonucleotides Used:

| SEQ ID NO | Motif | CMP ID NO | Compound |
|---|---|---|---|
| 5 | agttaaaggaggagacaaat | 5.1 | AGTTaaaggaggagacAAAT |
| 6 | tcagttaaaggaggagacaa | 6.1 | TCAgttaaaggaggagaCAA |
| 7 | ctcagttaaaggaggagaca | 7.1 | CTCagttaaaggaggagaCA |
| 8 | ctcagttaaaggaggagac | 8.1 | CTCagttaaaggaggaGAC |
| 9 | actcagttaaaggaggagac | 9.1 | ACTCagttaaaggaggagAC |
| 10 | actcagttaaaggaggaga | 10.1 | ACTCagttaaaggaggaGA |
| 11 | actcagttaaaggaggag | 11.1 | ACtcagttaaaggaGGAG |
| 12 | gatgactcagttaaaggagg | 12.1 | GAtgactcagttaaaggAGG |
| 13 | atgatgactcagttaaagga | 13.1 | ATGAtgactcagttaaagGA |
| 14 | tgatgactcagttaaagg | 14.1 | TGAtgactcagttaAAGG |
| 15 | gatgatgactcagttaaagg | 15.1 | GAtgatgactcagttaAAGG |
| 16 | gatgatgactcagttaaag | 16.1 | GATGatgactcagttaAAG |
| 17 | tatcgactgcattagttgg | 17.1 | TAT^{m}cgactgcattagttGG |
| 18 | gtatcgactgcattagttgg | 18.1 | Gtat^{m}cgactgcattagttGG |
| 19 | tcgactgcattagttg | 19.1 | TCGactgcattagTTG |
| 19 | tcgactgcattagttg | 19.2 | TCGactgcattagtTG |
| 19 | tcgactgcattagttg | 19.3 | TCGActgcattaGTTG |
| 20 | tatcgactgcattagttg | 20.1 | TAt^{m}cgactgcattaGTTG |
| 21 | gtatcgactgcattagttg | 21.1 | GTAt^{m}cgactgcattagtTG |
| 22 | tgtatcgactgcattagttg | 22.1 | TGtat^{m}cgactgcattagtTG |
| 23 | atcgactgcattagtt | 23.1 | ATCgactgcattaGTT |
| 23 | atcgactgcattagtt | 23.2 | ATCGactgcattAGTT |
| 23 | atcgactgcattagtt | 23.3 | ATCGactgcattaGTT |
| 24 | tatcgactgcattagtt | 24.1 | TATCgactgcattaGTT |
| 25 | gtatcgactgcattagtt | 25.1 | GTAT^{m}cgactgcattagTT |
| 26 | tgtatcgactgcattagtt | 26.1 | TGTat^{m}cgactgcattagTT |
| 27 | ttgtatcgactgcattagtt | 27.1 | TTGtat^{m}cgactgcattagTT |
| 28 | tatcgactgcattagt | 28.1 | TAT^{m}cgactgcattaGT |
| 28 | tatcgactgcattagt | 28.2 | TATCgactgcatTAGT |
| 29 | gtatcgactgcattagt | 29.1 | GTAT^{m}cgactgcattaGT |
| 30 | tgtatcgactgcattagt | 30.1 | TGTat^{m}cgactgcattaGT |
| 31 | gtatcgactgcattag | 31.1 | GTAt^{m}cgactgcatTAG |
| 31 | gtatcgactgcattag | 31.2 | GTAt^{m}cgactgcattAG |
| 31 | gtatcgactgcattag | 31.3 | GTAT^{m}cgactgcaTTAG |
| 32 | tgtatcgactgcattag | 32.1 | TGtat^{m}cgactgcaTTAG |
| 33 | ttgtatcgactgcattag | 33.1 | TTGtat^{m}cgactgcatTAG |
| 34 | attgtatcgactgcattag | 34.1 | ATtgtat^{m}cgactgcaTTAG |
| 35 | tgtatcgactgcatta | 35.1 | TGTat^{m}cgactgcaTTA |
| 35 | tgtatcgactgcatta | 35.2 | TGTAt^{m}cgactgcATTA |
| 36 | attgtatcgactgcatta | 36.1 | ATTGtat^{m}cgactgcaTTA |
| 37 | ttgtatcgactgcatt | 37.1 | TTGtat^{m}cgactgcaTT |
| 37 | ttgtatcgactgcatt | 37.2 | TTGtat^{m}cgactgCATT |
| 38 | attgtatcgactgcat | 38.1 | ATTgtat^{m}cgactgCAT |
| 38 | attgtatcgactgcat | 38.2 | ATTgtat^{m}cgactgcAT |
| 38 | attgtatcgactgcat | 38.3 | ATTGtat^{m}cgactGCAT |
| 39 | acgcattgtatcgact | 39.1 | ACGcattgtat^{m}cgACT |
| 39 | acgcattgtatcgact | 39.2 | ACGCattgtat^{m}cGACT |
| 40 | tacgcattgtatcgac | 40.1 | TACgcattgtat^{m}cGAC |
| 40 | tacgcattgtatcgac | 40.2 | TACGcattgtatCGAC |
| 41 | ctacgcattgtatcgac | 41.1 | CTa^{m}cgcattgtatCGAC |
| 42 | tctacgcattgtatcgac | 42.1 | TCTA^{m}cgcattgtat^{m}cgAC |
| 43 | atctacgcattgtatcgac | 43.1 | ATCta^{m}cgcattgtat^{m}cgAC |
| 44 | tatctacgcattgtatcgac | 44.1 | TAtcta^{m}cgcattgtatcGAC |
| 45 | ctacgcattgtatcga | 45.1 | CTA^{m}cgcattgtatCGA |
| 45 | ctacgcattgtatcga | 45.2 | CTACgcattgtaTCGA |
| 46 | tatctacgcattgtatcga | 46.1 | TAtcta^{m}cgcattgtatCGA |
| 47 | tctacgcattgtatcg | 47.1 | TCTa^{m}cgcattgtaTCG |
| 47 | tctacgcattgtatcg | 47.2 | TCTa^{m}cgcattgtatCG |
| 47 | tctacgcattgtatcg | 47.3 | TCTA^{m}cgcattgtATCG |
| 48 | atctacgcattgtatcg | 48.1 | ATCTa^{m}cgcattgtaTCG |
| 49 | tatctacgcattgtatcg | 49.1 | TATCta^{m}cgcattgtatCG |
| 50 | tctatctacgcattgtatcg | 50.1 | TCtatcta^{m}cgcattgtatCG |
| 51 | atctacgcattgtatc | 51.1 | ATCta^{m}cgcattgtATC |
| 51 | atctacgcattgtatc | 51.2 | ATCTa^{m}cgcattgTATC |
| 52 | tatctacgcattgtatc | 52.1 | TATcta^{m}cgcattgTATC |
| 53 | ctatctacgcattgtatc | 53.1 | CTatcta^{m}cgcattgTATC |
| 54 | tctatctacgcattgtatc | 54.1 | TCTatcta^{m}cgcattgtaTC |
| 55 | ttctatctacgcattgtatc | 55.1 | TTCtatcta^{m}cgcattgtaTC |
| 56 | tatctacgcattgtat | 56.1 | TATcta^{m}cgcattgTAT |
| 56 | tatctacgcattgtat | 56.2 | TATCta^{m}cgcattGTAT |
| 57 | ctatctacgcattgtat | 57.1 | CTAtcta^{m}cgcattGTAT |
| 58 | tctatctacgcattgtat | 58.1 | TCtatcta^{m}cgcattGTAT |
| 59 | ttctatctacgcattgtat | 59.1 | TTCtatcta^{m}cgcattgTAT |
| 60 | ctatctacgcattgta | 60.1 | CTAtcta^{m}cgcattGTA |
| 60 | ctatctacgcattgta | 60.2 | CTATcta^{m}cgcatTGTA |
| 61 | tctatctacgcattgta | 61.1 | TCTatcta^{m}cgcattGTA |
| 62 | ttctatctacgcattgta | 62.1 | TTCtatcta^{m}cgcattGTA |
| 63 | ttctatctacgcattgt | 63.1 | TTCtatcta^{m}cgcatTGT |
| 64 | tcttctatctacgcattgt | 64.1 | TCttctatcta^{m}cgcattGT |
| 65 | ttcttctatctacgcattgt | 65.1 | Ttcttctatcta^{m}cgcattGT |
| 66 | ttcttctatctacgcattg | 66.1 | TTCttctatcta^{m}cgcatTG |
| 67 | ttctatctacgcattg | 67.1 | TTCtatcta^{m}cgcaTTG |
| 68 | cttctatctacgcatt | 68.1 | CTTCtatcta^{m}cgCATT |
| 69 | tcttctatctacgcatt | 69.1 | TCTtctatcta^{m}cgCATT |
| 70 | ttcttctatctacgcatt | 70.1 | TTCTtctatcta^{m}cgcATT |
| 71 | tcttctatctacgcat | 71.1 | TCTTctatcta^{m}cgCAT |
| 72 | ttcttctatctacgcat | 72.1 | TTCTtctatcta^{m}cgCAT |
| 73 | cttcttctatctacgcat | 73.1 | CTTCttctatcta^{m}cgcAT |
| 74 | ttcttctatctacgca | 74.1 | TTCttctatctacGCA |
| 75 | cttcttctatctacgca | 75.1 | CTTCttctatcta^{m}cgCA |
| 76 | gcttcttctatctacgca | 76.1 | Gcttcttctatcta^{m}cgCA |
| 77 | cttcttctatctacgc | 77.1 | CTtcttctatctACGC |
| 78 | gcttcttctatctacg | 78.1 | GCTtcttctatctACG |
| 79 | cgtggggcttcttcta | 79.1 | CGTggggcttcttCTA |
| 80 | tgacttggagaaaagcacaa | 80.1 | TGacttggagaaaagcacAA |
| 81 | ctgacttggagaaaagcac | 81.1 | CtgacttggagaaaagcAC |
| 82 | agagtcatcgtgctcc | 82.1 | AGAgtcat^{m}cgtgcTCC |
| 83 | aagtactttaatagctcaaa | 83.1 | AAGTactttaatagctCAAA |
| 84 | aagtactttaatagctcaa | 84.1 | AAGTactttaatagcTCAA |
| 85 | gaagtactttaatagctcaa | 85.1 | GAAGtactttaatagctCAA |
| 86 | tactttaatagctcaa | 86.1 | TACTttaatagcTCAA |
| 87 | aagtactttaatagctca | 87.1 | AAGTactttaatagcTCA |
| 88 | gaagtactttaatagctca | 88.1 | GAAGtactttaatagcTCA |
| 89 | agaagtactttaatagctc | 89.1 | AGAAgtactttaatagCTC |
| 90 | aagaagtactttaatagctc | 90.1 | AAGAagtactttaatagCTC |
| 91 | gaagtactttaatagct | 91.1 | GAAGtactttaatAGCT |
| 92 | taagaagtactttaatagct | 92.1 | TAAgaagtactttaatAGCT |
| 93 | agaagtactttaatagc | 93.1 | AGAAgtactttaaTAGC |
| 94 | taagaagtactttaatagc | 94.1 | TAAGaagtactttaaTAGC |
| 95 | gtaagaagtactttaatagc | 95.1 | GTaagaagtactttaaTAGC |
| 96 | taagaagtactttaatag | 96.1 | TAAGaagtactttaATAG |
| 97 | gtaagaagtactttaatag | 97.1 | GTAAgaagtactttaATAG |
| 98 | tgtaagaagtactttaatag | 98.1 | TGTAagaagtactttaATAG |
| 99 | aatgtgtaagaagtacttt | 99.1 | AATGtgtaagaagtaCTTT |
| 100 | caatgtgtaagaagtacttt | 100.1 | CAATgtgtaagaagtaCTTT |
| 101 | atgtgtaagaagtactt | 101.1 | ATGTgtaagaagtACTT |
| 102 | aatgtgtaagaagtactt | 102.1 | AATGtgtaagaagtACTT |
| 103 | caatgtgtaagaagtactt | 103.1 | CAATgtgtaagaagtACTT |
| 104 | gcaatgtgtaagaagtactt | 104.1 | GCaatgtgtaagaagtACTT |
| 105 | atgtgtaagaagtact | 105.1 | ATGtgtaagaagtACT |
| 105 | atgtgtaagaagtact | 105.2 | ATGTgtaagaagTACT |
| 106 | gcaatgtgtaagaagtact | 106.1 | GCAAtgtgtaagaagtACT |
| 107 | aatgtgtaagaagtac | 107.1 | AATGtgtaagaaGTAC |
| 107 | aatgtgtaagaagtac | 107.2 | AATgtgtaagaaGTAC |
| 108 | caatgtgtaagaagtac | 108.1 | CAATgtgtaagaaGTAC |
| 109 | gcaatgtgtaagaagtac | 109.1 | GCAatgtgtaagaaGTAC |
| 110 | caatgtgtaagaagta | 110.1 | CAAtgtgtaagaaGTA |
| 110 | caatgtgtaagaagta | 110.2 | CAAtgtgtaagaAGTA |
| 110 | caatgtgtaagaagta | 110.3 | CAATgtgtaagaAGTA |
| 111 | gcaatgtgtaagaagta | 111.1 | GCAatgtgtaagaAGTA |
| 112 | gcaatgtgtaagaagt | 112.1 | GCAatgtgtaagaAGT |
| | | A | See below |
| | | B | See below |

For Compounds: Capital letters represent LNA nucleosides (beta-D-oxy LNA nucleosides were used), all LNA cytosines are 5-methyl cytosine, lower case letters represent DNA nucleosides, DNA cytosines preceded with a superscript ^{m} represent a 5-methyl C-DNA nucleoside. All internucleoside linkages are phosphorothioate internucleoside linkages. Compound A is disclosed as compound 143.1 and compound B is disclosed as compound 145.1 in EP16177508.5 and EP17170129.5, and are used as positive control compounds.

### Example 1. Testing In Vitro Efficacy of LNA Oligonucleotides in U251 Cell Line at a Single Concentration

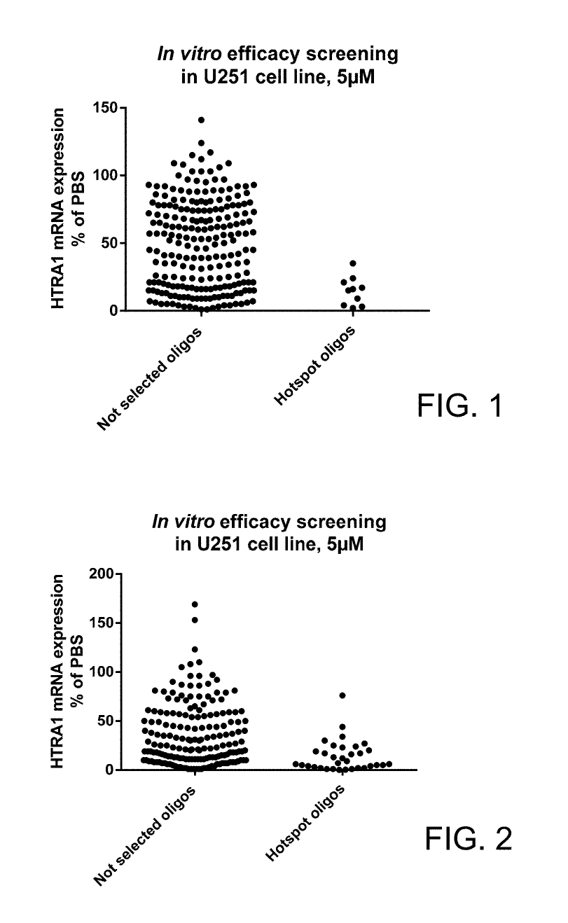

Identification of promising “hot spot” region for HTRA1. A library of n=231 HTRA1 LNA oligonucleotides were screened in U251 cell line at 5 μM, 6 days of treatment. From this library, we identified a series of active oligonucleotides targeting human HTRA1 pre-mRNA between position 53113-53384 as shown in FIG. 1 (SEQ ID NO 116 or 117).

Human glioblastoma U251 cell line was purchased from ECACC and maintained as recommended by the supplier in a humidified incubator at 37° C. with 5% CO₂. For assays, 15000 U251 cells/well were seeded in a 96 multi well plate in starvation media (media recommended by the supplier with the exception of 1% FBS instead of 10%). Cells were incubated for 24 hours before addition of oligonucleotides dissolved in PBS. Concentration of oligonucleotides: 5 μM. 3-4 days after addition of oligonucleotides, media was removed and new media (without oligonucleotide) was added. 6 days after addition of oligonucleotides, the cells were harvested. RNA was extracted using the PureLink Pro 96 RNA Purification kit (Ambion, according to the manufacturer's instructions). cDNA was then synthesized using M-MLT Reverse Transcriptase, random decamers RETROscript, RNase inhibitor (Ambion, according the manufacturer's instruction) with 100 mM dNTP set PCR Grade (Invitrogen) and DNase/RNase free Water (Gibco). For gene expressions analysis, qPCR was performed using TagMan Fast Advanced Master Mix (2×) (Ambion) in a doublex set up. Following TaqMan primer assays were used for qPCR: HTRA1, Hs01016151_m1 (FAM-MGB) and house keeping gene, TBP, Hs4326322E (VIC-MGB) from Life Technologies. n=2 independent biological replicates. The residual HTRA1 mRNA expression level in the table is shown as % of control (PBS-treated cells).

| SEQ ID NO | CMP ID NO | mRNA level |
|---|---|---|
| 19 | 19.1 | 16 |
| 31 | 31.1 | 2 |
| 38 | 38.1 | 9 |
| 47 | 47.1 | 3 |
| 78 | 78.1 | 4 |
| 79 | 79.1 | 21 |
| 82 | 82.1 | 35 |
| 107 | 107.1 | 17 |
| 110 | 110.1 | 24 |
| 112 | 112.1 | 15 |

### Example 2. Testing In Vitro Efficacy of LNA Oligonucleotides in U251 Cell Line at a Single Concentration

The “hot spot” region 53113-53384 described in Example 1 was further validated in a new library of n=210 HTRA1 LNA oligonucleotides that were screened in U251 cell line at 5 μM. n=33 LNA oligonucleotides were targeting human HTRA1 pre-mRNA between position 53113-53384 and these oligos were relatively active in comparison to the rest as shown in FIG. 2. The assay was performed as described in example 1. n=2 independent biological replicates. The residual HTRA1 mRNA expression level is shown in the table as % of control (PBS-treated cells).

| SEQ ID NO | CMP ID NO | mRNA level |
|---|---|---|
| 19 | 19.2 | 3 |
| 19 | 19.3 | 16 |
| 23 | 23.1 | 1 |
| 23 | 23.2 | 44 |
| 28 | 28.1 | 2 |
| 28 | 28.2 | 19 |
| 31 | 31.2 | 0.4 |
| 31 | 31.3 | 9 |
| 35 | 35.1 | 24 |
| 35 | 35.2 | 5 |
| 37 | 37.1 | 0.3 |
| 37 | 37.2 | 7 |
| 38 | 38.2 | 1 |
| 38 | 38.3 | 17 |
| 39 | 39.1 | 5 |
| 39 | 39.2 | 17 |
| 40 | 40.1 | 6 |
| 40 | 40.2 | 34 |
| 45 | 45.1 | 4 |
| 45 | 45.2 | 23 |
| 47 | 47.2 | 1 |
| 47 | 47.3 | 4 |
| 51 | 51.1 | 6 |
| 51 | 51.2 | 13 |
| 56 | 56.1 | 2 |
| 56 | 56.2 | 12 |
| 60 | 60.1 | 2 |
| 60 | 60.2 | 5 |
| 105 | 105.1 | 30 |
| 105 | 105.2 | 76 |
| 107 | 107.2 | 25 |
| 110 | 110.2 | 27 |
| 110 | 110.3 | 20 |

### Example 3. Testing In Vitro Efficacy of LNA Oligonucleotides in U251 and ARPE19 Cell Lines at a Single Concentration

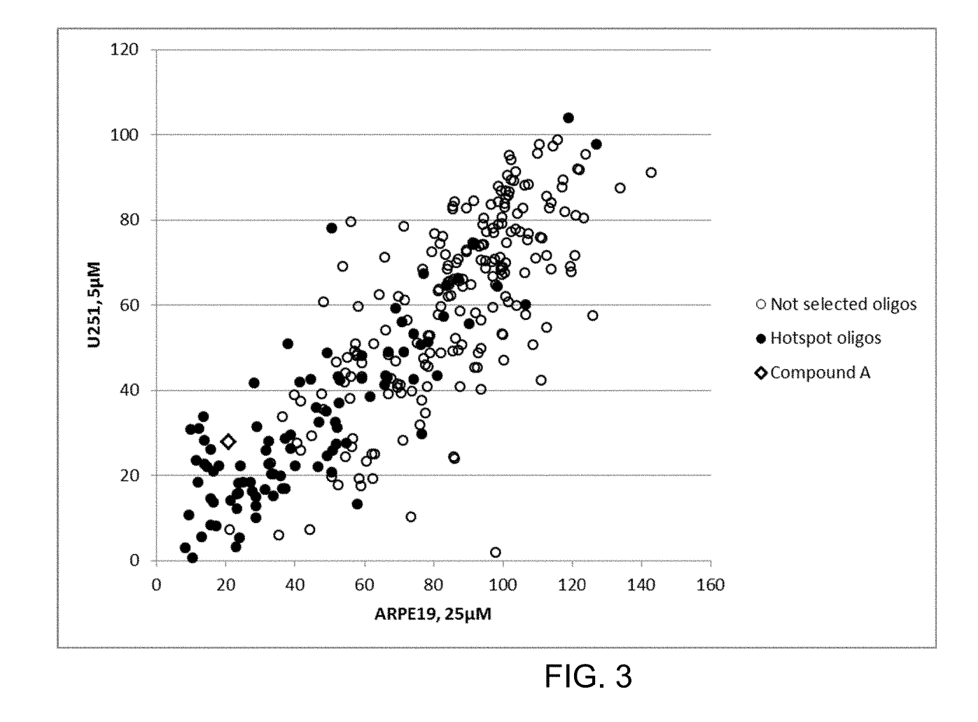

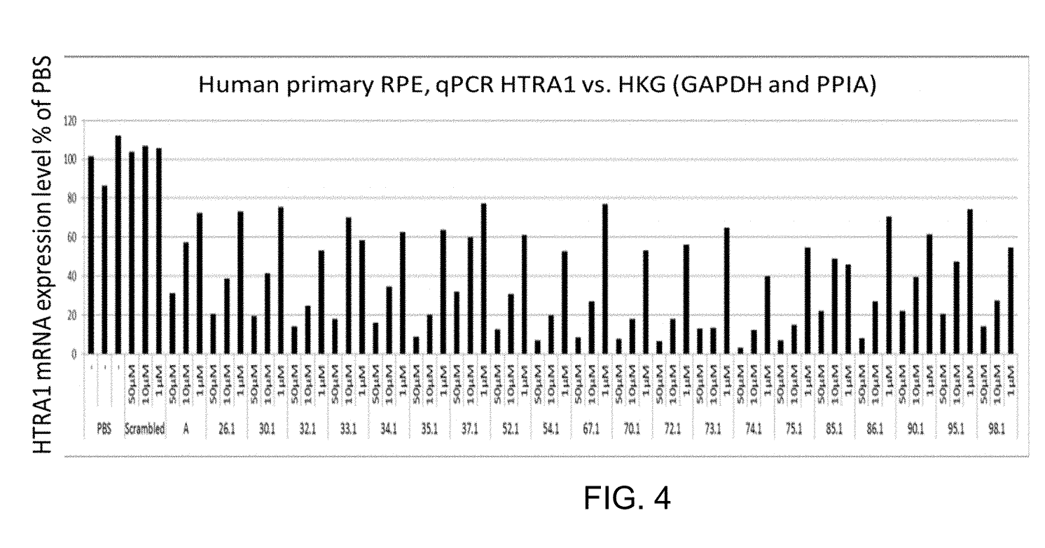

The “hot spot” region 53113-53384 described in Example 1 and 2 was further validated in a new library of n=305 HTRA1 LNA oligonucleotides that were screened in U251 and ARPE19 cell lines at 5 μM and 25 μM, respectively. n=95 LNA oligonucleotides were targeting human HTRA1 pre-mRNA between position 53113-53384 and these oligos were relatively active in comparison to the rest as shown in FIG. 3.

Human retinal pigmented epithelium ARPE19 cell line was purchased by from ATCC and maintained in DMEM-F12 (Sigma, D8437), 10% FBS, 1% pen/strep in a humidified incubator at 37° C. with 5% CO₂. The U251 cell line was described in example 1. For assays, 2000 U251 or ARPE19 cells/well were seeded in a 96 multi well plate in culture media recommended by the supplier. Cells were incubated for 2 hours before addition of oligonucleotides dissolved in PBS. Concentration of oligo was 5 and 25 μM in U251 and ARPE19 cells, respectively. 4 days after addition of oligonucleotides, the cells were harvested. RNA extraction was performed as described in example 1, cDNA synthesis and qPCR were performed using qScript XLT one-step RT-qPCR ToughMix Low ROX, 95134-100 (Quanta Biosciences). Following TaqMan primer assays were used for U251 and ARPE19 cells in a douplex set up: HTRA1, Hs01016151_m1 (FAM-MGB) and house keeping gene, GAPDH, Hs4310884E (VIC-MGB). All primer sets were purchased from Life Technologies. n=1 biological replicate. The relative HTRA1 mRNA expression level in the table is shown as % of control (PBS-treated cells).

| SEQ ID NO | CMP ID NO | ARPE19 mRNA level | U251 mRNA level |
|---|---|---|---|
| 5 | 5.1 | 90 | 56 |
| 6 | 6.1 | 107 | 60 |
| 7 | 7.1 | 92 | 74 |
| 8 | 8.1 | 83 | 57 |
| 9 | 9.1 | 98 | 64 |
| 10 | 10.1 | 77 | 67 |
| 11 | 11.1 | 71 | 56 |
| 12 | 12.1 | 81 | 43 |
| 13 | 13.1 | 84 | 65 |
| 14 | 14.1 | 36 | 20 |
| 15 | 15.1 | 37 | 29 |
| 16 | 16.1 | 55 | 28 |
| 17 | 17.1 | 53 | 43 |
| 18 | 18.1 | 69 | 59 |
| 20 | 20.1 | 41 | 42 |
| 21 | 21.1 | 24 | 22 |
| 22 | 22.1 | 38 | 51 |
| 23 | 23.3 | 53 | 37 |
| 24 | 24.1 | 52 | 27 |
| 25 | 25.1 | 27 | 18 |
| 26 | 26.1 | 16 | 26 |
| 27 | 27.1 | 28 | 42 |
| 29 | 29.1 | 24 | 16 |
| 30 | 30.1 | 18 | 22 |
| 31 | 31.2 | 23 | 3 |
| 32 | 32.1 | 14 | 23 |
| 33 | 33.1 | 11 | 23 |
| 34 | 34.1 | 14 | 34 |
| 35 | 35.1 | 8 | 3 |
| 36 | 36.1 | 12 | 18 |
| 37 | 37.1 | 24 | 5 |
| 41 | 41.1 | 51 | 26 |
| 42 | 42.1 | 39 | 26 |
| 43 | 43.1 | 53 | 42 |
| 44 | 44.1 | 67 | 49 |
| 46 | 46.1 | 59 | 43 |
| 47 | 47.2 | 16 | 8 |
| 48 | 48.1 | 23 | 15 |
| 49 | 49.1 | 39 | 29 |
| 50 | 50.1 | 45 | 42 |
| 51 | 51.1 | 14 | 28 |
| 52 | 52.1 | 15 | 22 |
| 53 | 53.1 | 32 | 23 |
| 54 | 54.1 | 12 | 31 |
| 55 | 55.1 | 46 | 36 |
| 56 | 56.1 | 9 | 11 |
| 57 | 57.1 | 62 | 38 |
| 58 | 58.1 | 77 | 30 |
| 59 | 59.1 | 29 | 31 |
| 60 | 60.1 | 47 | 22 |
| 61 | 61.1 | 25 | 18 |
| 62 | 62.1 | 32 | 26 |
| 63 | 63.1 | 32 | 17 |
| 64 | 64.1 | 67 | 43 |
| 65 | 65.1 | 51 | 78 |
| 66 | 66.1 | 24 | 18 |
| 67 | 67.1 | 11 | 0.7 |
| 68 | 68.1 | 37 | 17 |
| 69 | 69.1 | 36 | 17 |
| 70 | 70.1 | 23 | 12 |
| 71 | 71.1 | 34 | 15 |
| 72 | 72.1 | 16 | 15 |
| 73 | 73.1 | 16 | 14 |
| 74 | 74.1 | 17 | 8 |
| 75 | 75.1 | 29 | 13 |
| 76 | 76.1 | 74 | 43 |
| 77 | 77.1 | 58 | 13 |
| 80 | 80.1 | 127 | 98 |
| 81 | 81.1 | 119 | 104 |
| 83 | 83.1 | 49 | 49 |
| 84 | 84.1 | 52 | 31 |
| 85 | 85.1 | 29 | 10 |
| 86 | 86.1 | 13 | 5 |
| 87 | 87.1 | 32 | 28 |
| 88 | 88.1 | 29 | 15 |
| 89 | 89.1 | 28 | 16 |
| 90 | 90.1 | 21 | 14 |
| 91 | 91.1 | 74 | 53 |
| 92 | 92.1 | 76 | 51 |
| 93 | 93.1 | 40 | 22 |
| 94 | 94.1 | 33 | 20 |
| 95 | 95.1 | 10 | 31 |
| 96 | 96.1 | 49 | 35 |
| 97 | 97.1 | 34 | 20 |
| 98 | 98.1 | 16 | 21 |
| 99 | 99.1 | 66 | 43 |
| 100 | 100.1 | 51 | 21 |
| 101 | 101.1 | 87 | 66 |
| 102 | 102.1 | 52 | 32 |
| 103 | 103.1 | 49 | 24 |
| 104 | 104.1 | 79 | 51 |
| 106 | 106.1 | 71 | 49 |
| 108 | 108.1 | 47 | 32 |
| 109 | 109.1 | 59 | 48 |
| 111 | 111.1 | 66 | 41 |
| A | A | 21 | 28 |

### Example 4. Testing In Vitro Potency and Efficacy of Selected Compounds in U251 and ARPE19 Cell Lines in a Dose Response Curve

The U251 and ARPE19 cell lines were described in example 1 and 3, respectively. The U251 assay was performed as described in Example 1. The ARPE19 assay was performed as follows: 5000 ARPE19 cells/well were seeded in a 96 multi well plate in culture media recommended by the supplier (with the exception of 5% FBS instead of 10%). Cells were incubated for 2 hour before addition of oligonucleotides dissolved in PBS. Concentration of oligonucleotides: from 50 μM, half-log dilution, 8 points. 4 days after addition of oligonucleotides, the cells were harvested. RNA extraction, cDNA synthesis and qPCR were performed as described in Example 1. n=2 independent biological replicates. The EC50 value and the residual HTRA1 mRNA level at 50 μM are shown in the table as % of control (PBS).

| | | ARPE19 | | U251 | |
|---|---|---|---|---|---|
| SEQ ID | CMP ID | EC50 | mRNA level | EC50 | mRNA level |
| NO | NO | (μM) | at max KD | (μM) | at max KD |
| 19 | 19.2 | 2.3 | 54 | 0.6 | 3 |
| 31 | 31.2 | 2.3 | 12 | 0.40 | 0.2 |
| 37 | 37.1 | 4.0 | 11 | 0.46 | 0.2 |
| 38 | 38.2 | 7.4 | 19 | 0.70 | 0.2 |
| 47 | 47.2 | 4.6 | 8 | 0.62 | 0.2 |
| 23 | 23.1 | 6.8 | 25 | 0.80 | 1 |
| 35 | 35.1 | 3.5 | 4 | 0.38 | 0.1 |

### Example 5, Testing In Vitro Potency and Efficacy of Selected Compounds in U251 and ARPE19 Cell Lines in a Dose Response Curve

The assays were performed as described in Example 3. Concentration of oligonucleotides: from 50 μM, half-log dilution, 8 points. n=2 and n=1 independent biological replicates for U251 and ARPE19, respectively. The EC50 value and the residual HTRA1 mRNA level at 50 μM are shown in the table as % of control (PBS).

| | | ARPE19 | | U251 | |
|---|---|---|---|---|---|
| SEQ ID | CMP ID | EC50 | mRNA level | EC50 | mRNA level at |
| NO | NO | (μM) | at max KD | (μM) | max KD |
| 31 | 31.2 | 3.2 | 15 | 0.90 | 0.38 |
| 37 | 37.1 | 11 | 22 | 1.3 | 0.75 |
| 47 | 47.2 | 2.8 | 13 | 0.89 | 0.83 |
| 35 | 35.1 | 2.6 | 8.3 | 0.79 | 0.40 |
| 85 | 85.1 | 8.2 | 24 | 0.48 | 3.6 |
| 90 | 90.1 | 3.3 | 16 | 0.50 | 2.2 |
| 95 | 95.1 | 0.55 | 28 | 1.0 | 4.1 |
| 98 | 98.1 | 1.7 | 24 | 0.86 | 4.5 |
| 30 | 30.1 | 1.2 | 20 | 1.00 | 2.2 |
| 32 | 32.1 | 1.7 | 22 | 1.6 | 1.4 |
| 26 | 26.1 | 1.1 | 14 | 1.4 | 0.45 |
| 33 | 33.1 | 0.75 | 28 | 0.66 | 0.63 |
| 34 | 34.1 | 0.44 | 21 | 0.80 | 0.35 |
| 36 | 36.1 | 5.2 | 28 | 1.1 | 0.80 |
| 52 | 52.1 | 2.1 | 28 | 1.1 | 1.1 |
| 54 | 54.1 | 0.79 | 25 | 0.62 | 1.4 |
| 72 | 72.1 | 2.9 | 33 | 0.71 | 1.7 |
| 70 | 70.1 | 1.9 | 36 | 0.52 | 1.5 |
| 74 | 74.1 | 0.78 | 24 | 0.35 | 1.1 |
| 73 | 73.1 | 0.78 | 11 | 0.59 | 0.33 |
| 75 | 75.1 | 1.7 | 22 | 0.60 | 0.80 |
| 86 | 86.1 | 1.7 | 6.5 | 0.47 | 0.65 |
| 67 | 67.1 | 0.59 | 4.3 | 0.38 | 0.23 |
| A | A | 6.5 | 24 | 1.2 | 3.6 |
| B | B | 8.1 | 30 | 0.79 | 4.2 |

### Example 6. Testing In Vitro Potency and Efficacy of Selected Compounds in U251 Cell Line in a Dose Response Curve

The assay was performed as described in Example 3. Concentration of oligonucleotides: from 50 μM, half-log dilution, 8 points. n=2 independent biological replicates. The EC50 value and the residual HTRA1 mRNA level at 50 μM are shown in the table as % of control (PBS).

U251

| | | EC50 | mRNA level |
|---|---|---|---|
| SEQ ID NO | CMP ID NO | (μM) | at max KD |
| 38 | 38.1 | 3.3 | 3 |
| 78 | 78.1 | 0.58 | 2 |
| 31 | 31.2 | 1.2 | 0.4 |
| 37 | 37.1 | 1.6 | 0.6 |
| 47 | 47.2 | 0.91 | 0.6 |
| 35 | 35.1 | 0.52 | 0.3 |
| 39 | 39.1 | 0.82 | 3 |
| 40 | 40.1 | 1.3 | 4 |
| 45 | 45.1 | 0.89 | 3 |
| 51 | 51.1 | 2.7 | 2 |
| 56 | 56.1 | 2.7 | 1 |
| 60 | 60.1 | 2.1 | 1 |
| 37 | 37.2 | 8.0 | 24 |
| 31 | 31.3 | 2.8 | 10 |
| 35 | 35.2 | 1.3 | 4 |
| 47 | 47.3 | 0.86 | 4 |
| 60 | 60.2 | 1.3 | 3 |
| 26 | 26.1 | 0.52 | 1 |
| 73 | 73.1 | 0.24 | 0.7 |
| 86 | 86.1 | 0.27 | 0.9 |
| 67 | 67.1 | 0.46 | 0.2 |
| A | A | 1.1 | 3.1 |
| B | B | 1.2 | 3.3 |

### Example 7. Testing In Vitro Potency and Efficacy of Selected Compounds in U251 Cell Line in a Dose Response Curve

The ARPE19 cell line was described in example 3. For assays, ARPE19 cells, 24000 cells/well were seeded in 100 μL in a 96 multi well plate in starvation media (culture media as recommended by the supplier with the exception of 1% FBS instead of 10%). Cells were incubated for 2 hour before addition of oligonucleotides dissolved in PBS. Concentration of oligonucleotides: from 50 μM, half-log dilution, 8 points. At day 4 and 7 after addition of oligonucleotide compounds 75 μL fresh starvation media without oligonucleotides was added to the cells (without removing the old media). RNA extraction, cDNA synthesis and qPCR were performed as described in Example 3. n=2 independent biological replicates. The EC50 value and the residual HTRA1 mRNA level at 50 μM are shown in the table as % of control (PBS).

ARPE19

| | | EC50 | mRNA level |
|---|---|---|---|
| SEQ ID NO | CMP ID NO | (μM) | at max KD |
| 30 | 30.1 | 0.31 | 1 |
| 33 | 33.1 | 0.60 | 0.5 |
| 35 | 35.1 | 0.58 | 1 |
| 35 | 35.2 | 2.7 | 4 |
| 36 | 36.1 | 0.97 | 2 |
| 37 | 37.1 | 1.0 | 4 |
| 40 | 40.1 | 3.8 | 21 |
| 45 | 45.1 | 1.6 | 3 |
| 56 | 56.1 | 5.8 | 2 |
| 67 | 67.1 | 0.84 | 1 |
| 73 | 73.1 | 0.36 | 2 |
| 86 | 86.1 | 0.59 | 4 |
| 90 | 90.1 | 0.75 | 5 |
| 95 | 95.1 | 0.74 | 3 |
| A | A | 1.3 | 1.9 |
| B | B | 0.84 | 1.5 |

### Example 8

### Testing In Vitro Efficacy in Human Primary RPE Cells.

Human primary Retinal Pigmented Epithelium (hpRPE) cells were purchased from Sciencell (Cat#6540). For assays, 5000 hpRPE cells/well were seeded in a Laminin (Laminin 521, BioLamina Cat# LN521-03) coated 96 multi well plate in culture media (EpiCM, Sciencell Cat#4101). They were expanded with this media for one week and differentiated using the following media for 2 weeks: MEM Alpha media (Sigma Cat# M-4526) supplemented with N1 supplement (Sigma Cat# N-6530), Glutamine-Penicillin-Streptomycin (Sigma Cat# G-1146), Non Essential Amino Acid (NEAA, Sigma Cat# M-7145), Taurine (Sigma Cat# T-0625), Hydrocortisone (Sigma Cat# H-03966), Triiodo-thyronin (Sigma Cat# T-5516) and Bovine Serum Albumin (BSA, Sigma Cat# A-9647). Cells were cultured in a humidified incubator at 37° C. with 5% CO₂.

On the day of the experiment, cells were incubated for 1 hour with fresh differentiation media before addition of oligonucleotides. These were dissolved in PBS and applied on cells at day 0 and day 4. On day 7, the media was changed, and on day 10 cells were harvested with 50 μl of RLT buffer with β-mercapto-ethanol (Qiagen Cat#79216). The extraction of the RNA was performed according to the user's manual of the Qiagen RNeasy Mini Kit (Cat#74104; Lot 151048073) including DNase I treatment (Cat#79254; Lot 151042674). RNA quality control was performed with the Agilent Bioanalyzer Nano Kit (Agilent; Cat#5067-1511; Lot 1446). Reverse transcription of total RNA into cDNA (cDNA synthesis) was performed using the High Capacity cDNA Reverse Transcription Kit (based on random hexamer oligonucleotides), according to the manufacturer's instructions (Thermo Fisher Scientific, Cat#4368814; Lot 00314158). The measurement of the cDNA samples was carried out in triplicates, in a 384-well plate format on the 7900HT real-time PCR instrument (Thermo Fisher Scientific). The following TaqMan primer assays were used for qPCR: HTRA1, Hs01016151_m1 and Hs00170197_m1, housekeeping genes, GAPDH, Hs99999905_m1 and PPIA, Hs99999904_m1, from Life Technologies. n=3 biological replicates. The residual HTRA1 mRNA expression level is shown in FIG. 4 and the following table as % of control (PBS).

| | | mRNA level | | |
|---|---|---|---|---|
| SEQ ID NO | CMP ID NO | 50 μM | 10 μM | 1 μM |
| 37 | 37.1 | 32 | 60 | 77 |
| 35 | 35.1 | 9 | 20 | 64 |
| 85 | 85.1 | 22 | 49 | 46 |
| 90 | 90.1 | 22 | 39 | 61 |
| 95 | 95.1 | 20 | 47 | 74 |
| 98 | 98.1 | 14 | 27 | 55 |
| 30 | 30.1 | 19 | 41 | 75 |
| 32 | 32.1 | 14 | 25 | 53 |
| 26 | 26.1 | 21 | 39 | 73 |
| 33 | 33.1 | 18 | 70 | 58 |
| 34 | 34.1 | 16 | 35 | 63 |
| 52 | 52.1 | 13 | 31 | 61 |
| 54 | 54.1 | 7 | 20 | 53 |
| 72 | 72.1 | 7 | 18 | 56 |
| 70 | 70.1 | 8 | 18 | 53 |
| 74 | 74.1 | 3 | 12 | 40 |
| 73 | 73.1 | 13 | 13 | 65 |
| 75 | 75.1 | 7 | 15 | 55 |
| 86 | 86.1 | 8 | 27 | 70 |
| 67 | 67.1 | 8 | 27 | 77 |
| A | A | 31 | 57 | 72 |

### Example 9. Cynomolgus Monkey In Vivo Pharmacokinetics and Pharmacodynamics Study, 21 Days of Treatment, Intravitreal (IVT) Injection, Single Dose

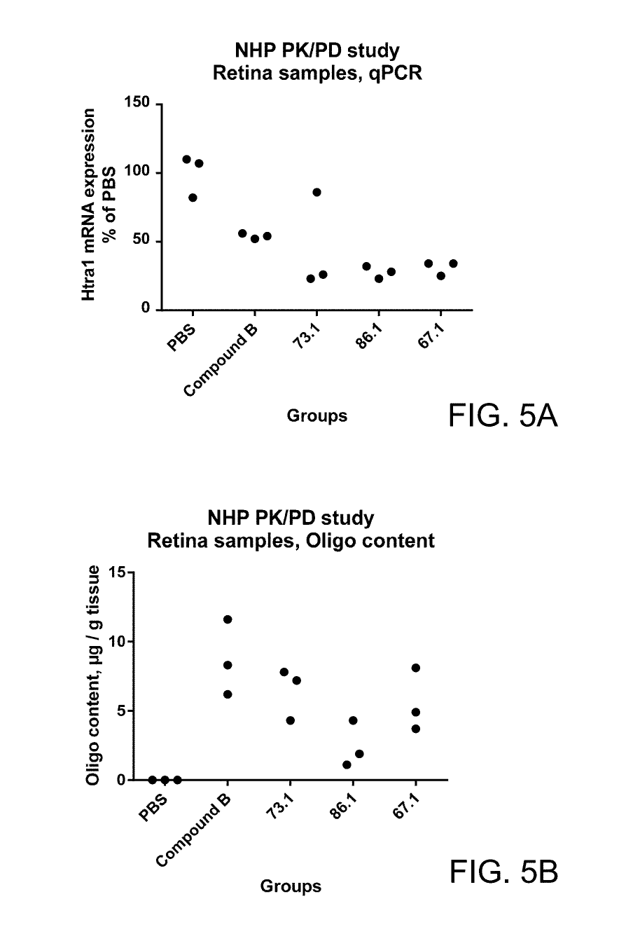

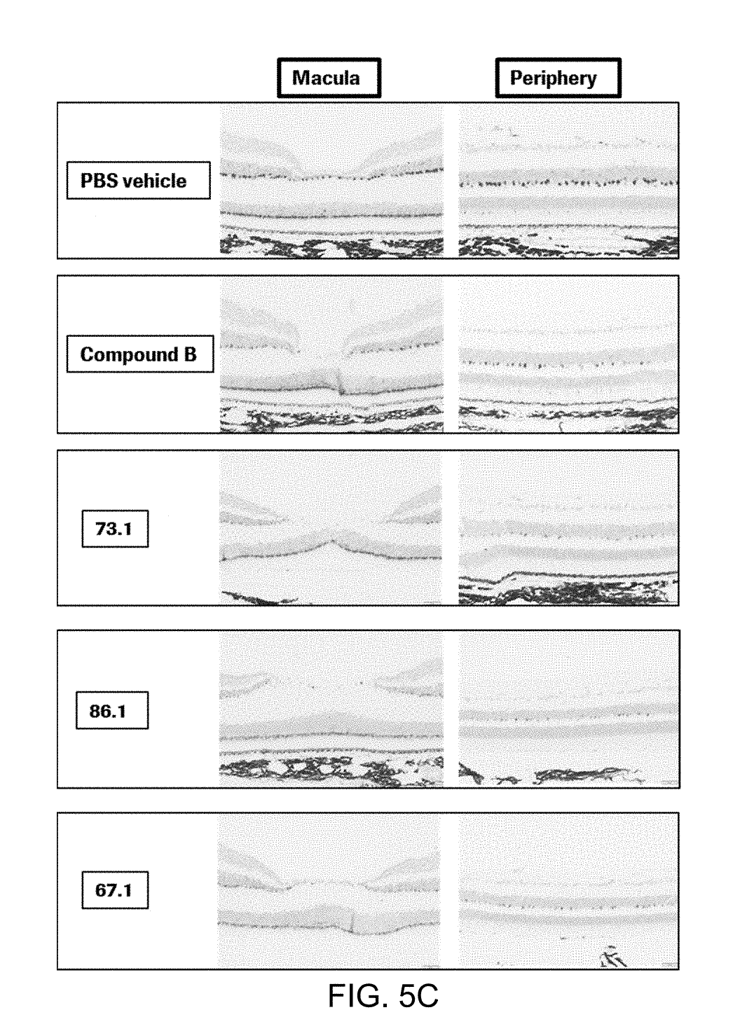

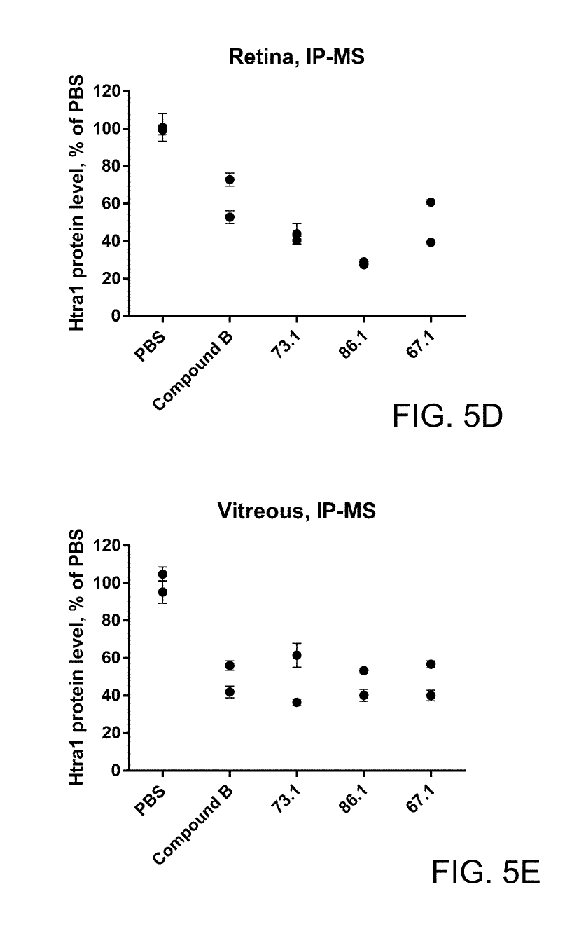

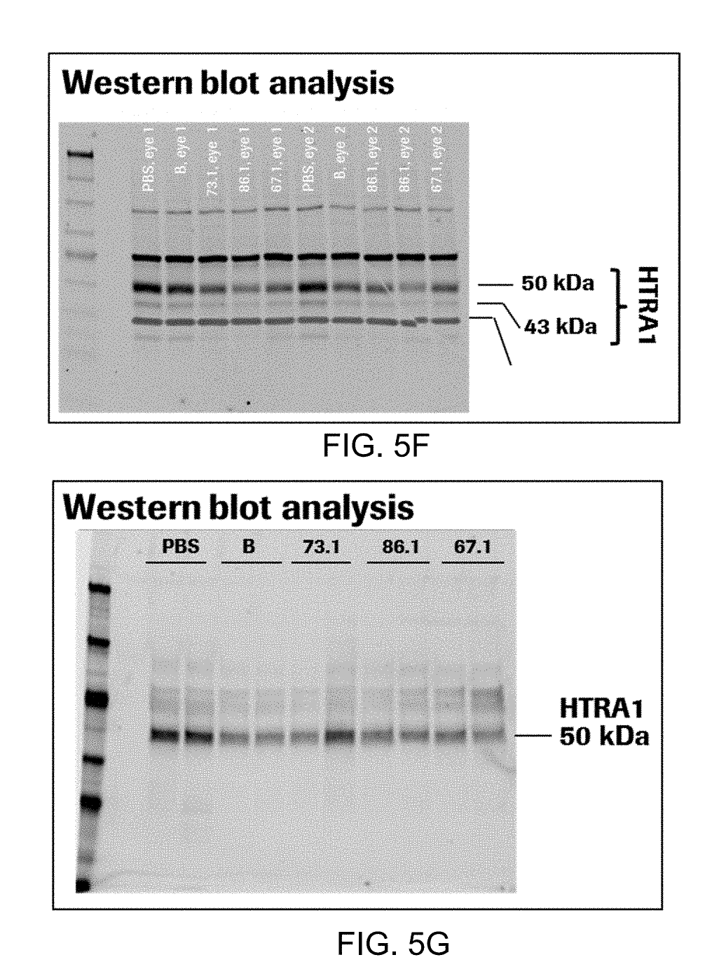

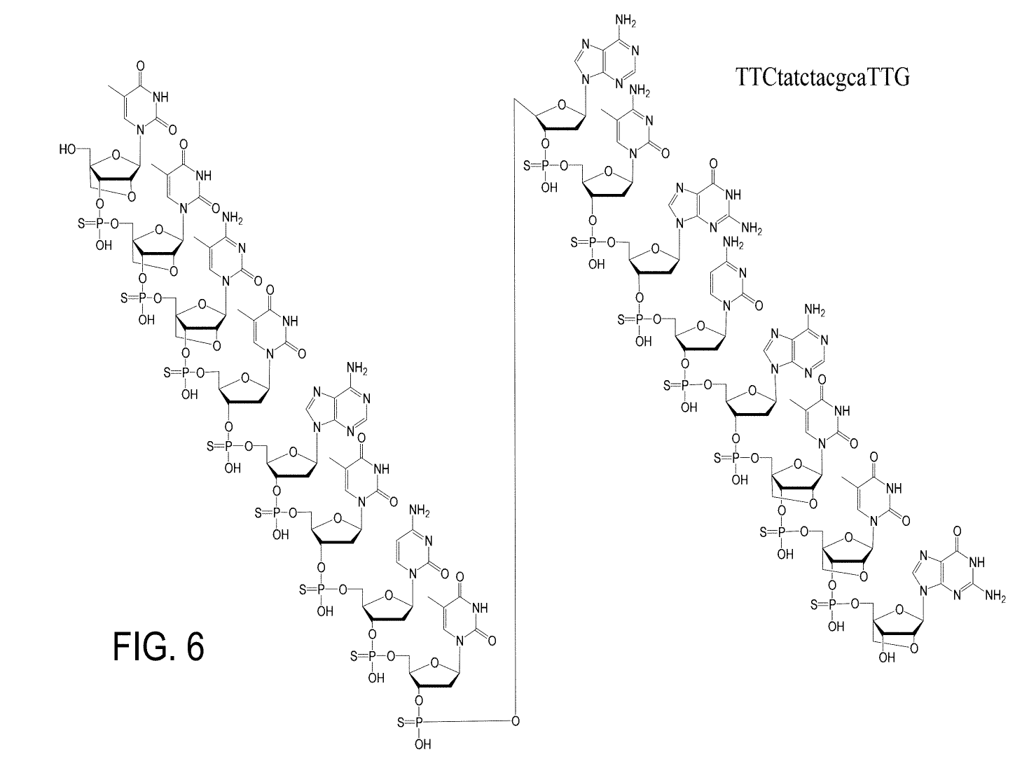

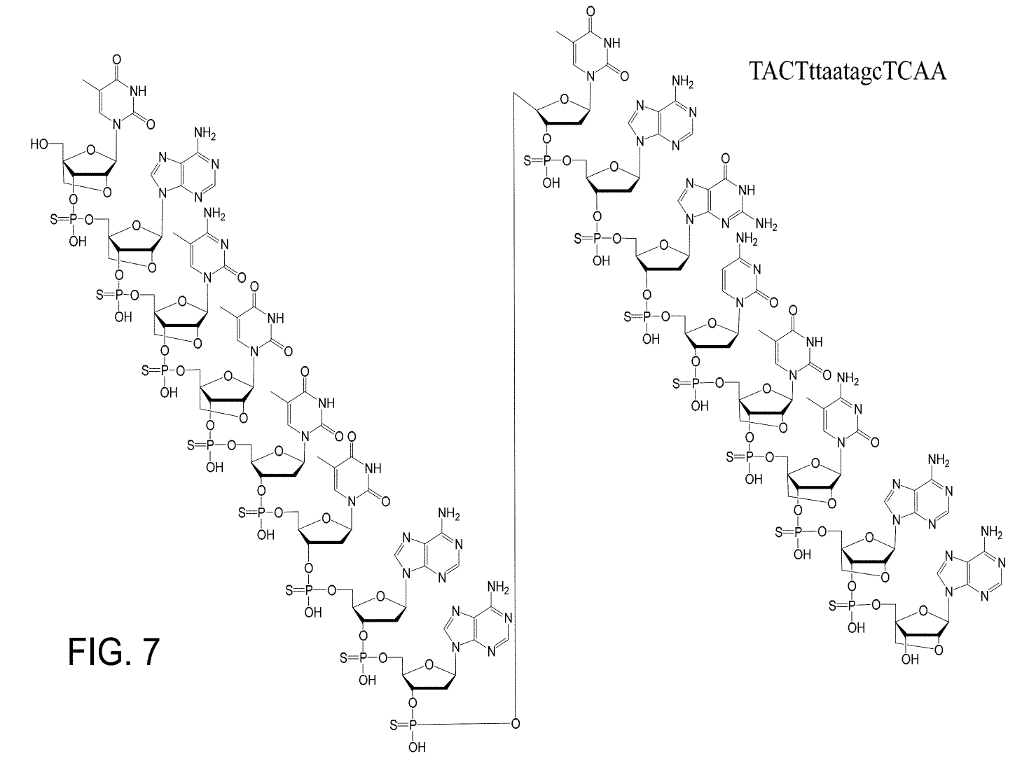

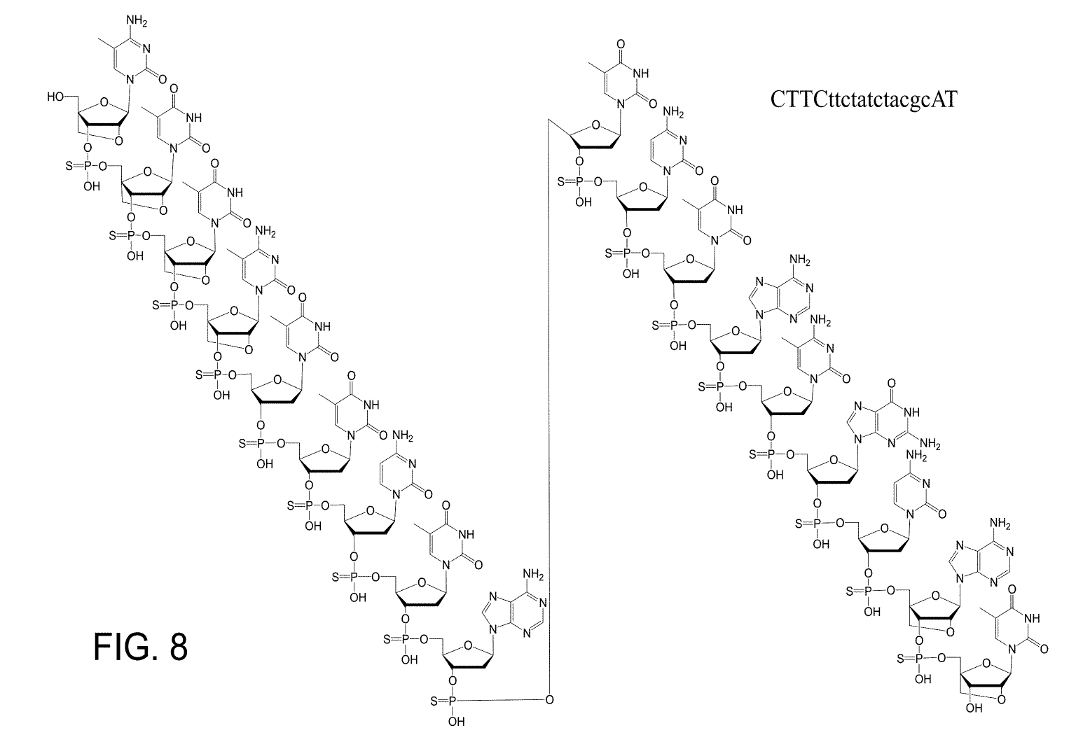

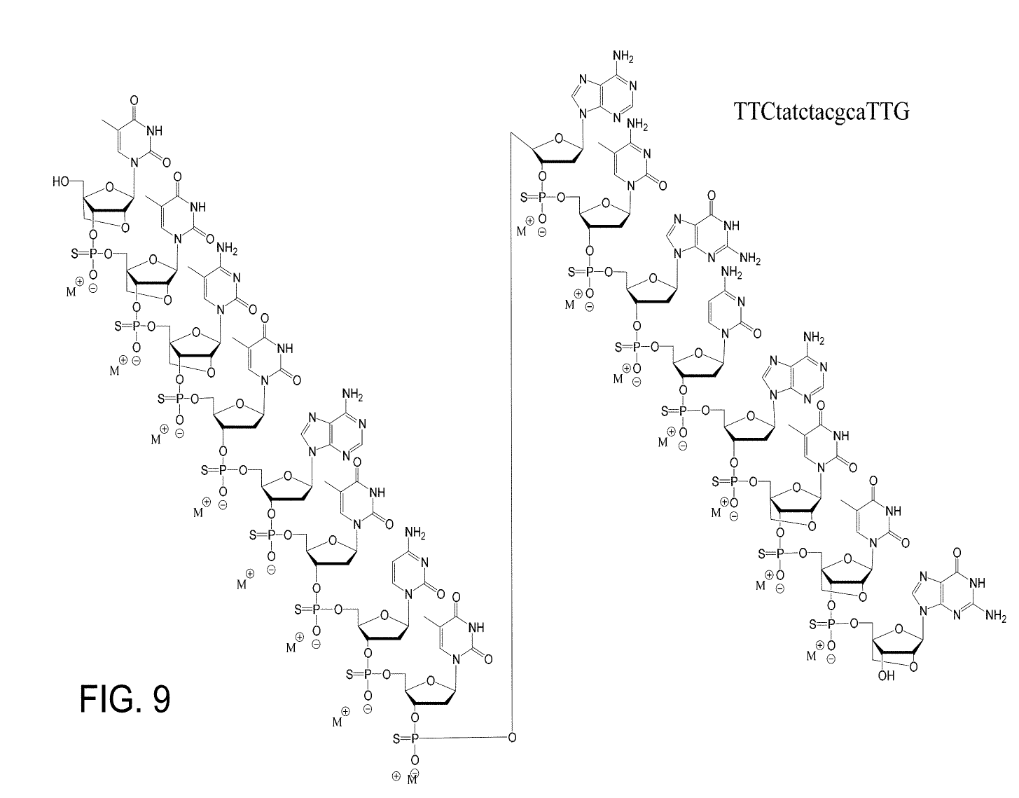

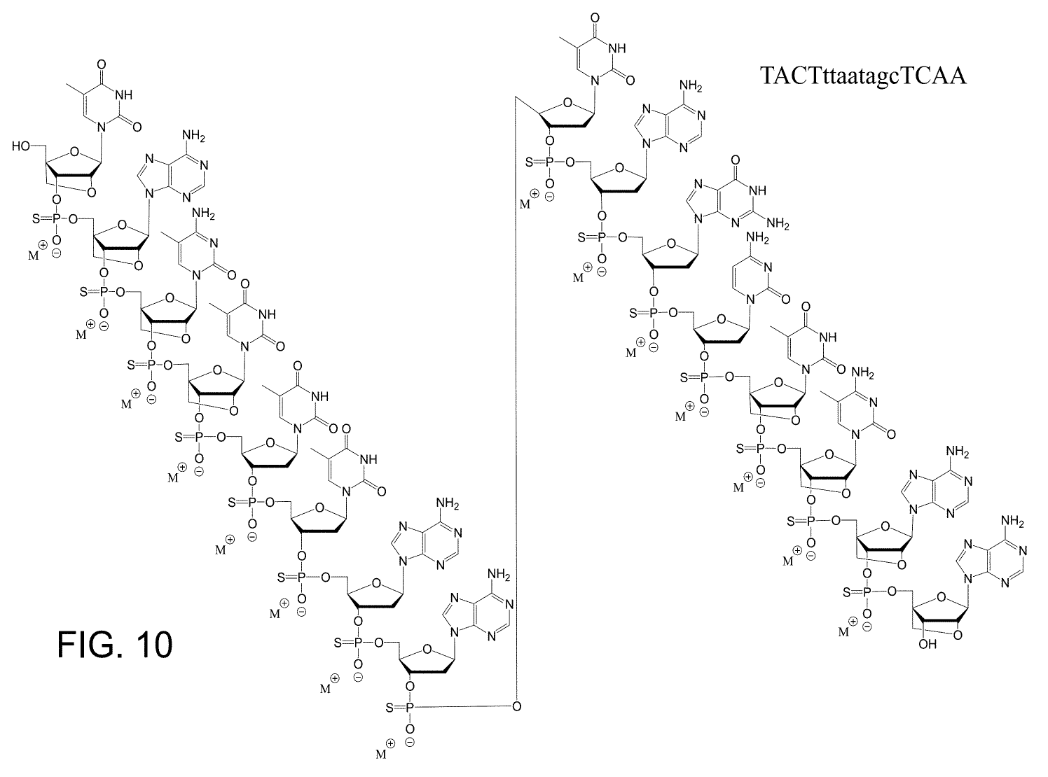

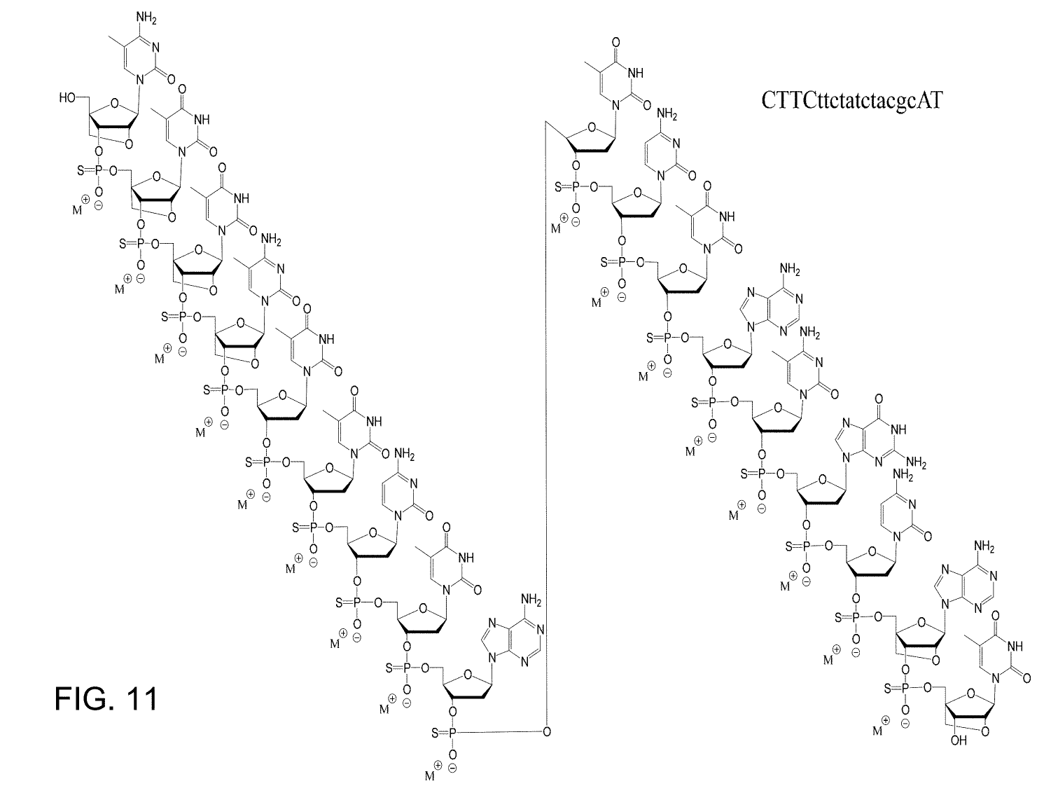

Knock down was observed for 3 HTRA1 LNA oligonucleotides targeting the “hotspot” in human HTRA1 pre-mRNA between position 53113-53384 both at mRNA in the retina and at protein level in the retina and in the vitreous (see FIG. 5)

### Animals

All experiments were performed on Cynomolgus monkeys (Macaca fascicularis).

Four animals were included in each group of the study, 20 in total.

### Compounds and Dosing Procedures

Buprenorphine analgesia was administered prior to, and two days after test compound injection. The animals were anesthetized with an intramuscular injection of ketamine and xylazine. The test item and negative control (PBS) were administered intravitreally in both eyes of anesthetized animals (50 μL per administration) on study day 1 after local application of tetracaine anesthetic.

### Euthanasia

At the end of the in-life phase (Day 22) all monkeys were euthanized by intraperitoneal an overdose injection of pentobarbital.

Oligo Content Measurement and Quantification of Htra1 RNA Expression by qPCR

Immediately after euthanasia, eye tissues were quickly and carefully dissected out on ice and stored at −80° C. until shipment. Retina sample was lysed in 700 μL MagNa Pure 96 LC RNA Isolation Tissue buffer and homogenized by adding 1 stainless steel bead per 2 ml tube 2×1.5 min using a precellys evolution homogenizer followed by 30 min incubation at RT. The samples were centrifuged, 13000 rpm, 5 min. Half was set aside for bioanalysis and for the other half, RNA extraction was continued directly.

For bioanalysis, the samples were diluted 10-50 fold for oligo content measurements with a hybridization ELISA method. A biotinylated LNA-capture probe and a digoxigenin-conjugated LNA-detection probe (both 35 nM in 5×SSCT, each complementary to one end of the LNA oligonucleotide to be detected) was mixed with the diluted homogenates or relevant standards, incubated for 30 minutes at RT and then added to a streptavidine-coated ELISA plates (Nunc cat. no. 436014).

The plates were incubated for 1 hour at RT, washed in 2×SSCT (300 mM sodium chloride, 30 mM sodium citrate and 0,05% v/v Tween-20, pH 7.0) The captured LNA duplexes were detected using an anti-DIG antibodies conjugated with alkaline phosphatase (Roche Applied Science cat. No. 11093274910) and an alkaline phosphatase substrate system (Blue Phos substrate, KPL product code 50-88-00). The amount of oligo complexes was measured as absorbance at 615 nm on a Biotek reader.

For RNA extraction, cellular RNA large volume kit (05467535001, Roche) was used in the MagNA Pure 96 system with the program: Tissue FF standard LV3.1 according to the instructions of the manufacturer, including DNAse treatment. RNA quality control and concentration were measured with an Eon reader (Biotek). The RNA concentration was normalized across samples, and subsequent cDNA synthesis and qPCR was performed in a one-step reaction using qScript XLT one-step RT-qPCR ToughMix Low ROX, 95134-100 (Quanta Biosciences). The following TaqMan primer assays were used in singplex reactions: Htra1, Mf01016150_, Mf01016152_m1 and Rh02799527_m1 and housekeeping genes, ARFGAP2, Mf01058488_g1 and Rh01058485_m1, and ARL1, Mf02795431_m1, from Life Technologies. The qPCR analyses were run on a ViiA7 machine (Life Technologies).

Eyes/group: n=3 eyes. Each eye was treated as an individual sample. The relative Htra1 mRNA expression level is shown as % of control (PBS).

### Histology

Eyeballs were removed and fixed in 10% neutral buffered formalin for 24 hours, trimmed and embedded in paraffin.

For ISH analysis, sections of formalin-fixed, paraffin-embedded cyno retina tissue 4 μm thick were processed using the fully automated Ventana Dicovery ULTRA Staining Module (Procedure: mRNA Discovery Ultra Red 4.0-v0.00.0152) using the RNAscope 2.5 VS Probe-Mmu-HTRA1, REF 486979, Advanced Cell Diagnostics, Inc., Chromogen used is Fastred, Hematoxylin H counterstain.

### HTRA1 Protein Quantification Using a Plate-Based Immunoprecipitation Mass Spectrometry (IP-MS) Approach

### Sample Preparation, Retina

Retinas were homogenized in 4 volumes (w/v) of RIPA buffer (50 mM Tris-HCl, pH 7.4, 150 mM NaCl, 0.25% deoxycholic acid, 1% NP-40, 1 mM EDTA, Millipore) with protease inhibitors (Complete EDTA-free, Roche) using a Precellys 24 (5500, 15 s, 2 cycles). Homogenates were centrifuged (13,000 rpm, 3 min) and the protein contents of the supernatants determined (Pierce BCA protein assay)

### Sample Preparation, Vitreous

Vitreous humors (300 μl) were diluted with 5×RIPA buffer (final concentration: 50 mM Tris-HCl, pH 7.4, 150 mM NaCl, 0.25% deoxycholic acid, 1% NP-40, 1 mM EDTA) with protease inhibitors (Complete EDTA-free, Roche) and homogenized using a Precellys 24 (5500, 15 s, 2 cycles). Homogenates were centrifuged (13,000 rpm, 3 min) and the protein contents of the supernatants determined (Pierce BCA protein assay)

### Plate-Based HTRA1 Immunoprecipitation and Tryptic Digest

A 96 well plate (Nunc MaxiSorp) was coated with anti-HTRA1 mouse monoclonal antibody (R&D MAB2916, 500 ng/well in 50 μl PBS) and incubated overnight at 4° C. The plate was washed twice with PBS (200 μl) and blocked with 3% (w/v) BSA in PBS for 30 min at 20° C. followed by two PBS washes. Samples (75 μg retina, 100 μg vitreous in 50 μl PBS) were randomized and added to the plate followed by overnight incubation at 4° C. on a shaker (150 rpm). The plate was then washed twice with PBS and once with water. 10 mM DTT in 50 mM TEAB (30 μl) were then added to each well followed by incubation for 1 h at 20° C. to reduce cysteine sulfhydryls. 150 mM iodoacetamide in 50 mM TEAB (5 μl) were then added to each well followed by incubation for 30 min at 20° C. in the dark in order to block cysteine sulfhydryls. 10 μl Digestion solution were added to each well (final concentrations: 1.24 ng/μl trypsin, 20 fmol/μl BSA peptides, 26 fmol/μl isotope-labeled HTRA1 peptides, 1 fmol/μl iRT peptides, Biognosys) followed by incubation overnight at 20° C.

### HTRA1 Peptide Quantification by Targeted Mass Spectrometry (Selected Reaction Monitoring, SRM)

Mass spectrometry analysis was performed on an Ultimate RSLCnano LC coupled to a TSQ Quantiva triple quadrupole mass spectrometer (Thermo Scientific). Samples (20 μL) were injected directly from the 96 well plate used for IP and loaded at 5 μL/min for 6 min onto a Acclaim Pepmap 100 trap column (100 μm×2 cm, C18, 5 μm, 100 Å, Thermo Scientific) in loading buffer (0.5% v/v formic acid, 2% v/v ACN). Peptides were then resolved on a PepMap Easy-SPRAY analytical column (75 μm×15 cm, 3 μm, 100 Å, Thermo Scientific) with integrated electrospray emitter heated to 40° C. using the following gradient at a flow rate of 250 nL/min: 6 min, 98% buffer A (2% ACN, 0.1% formic acid), 2% buffer B (ACN+0.1% formic acid); 36 min, 30% buffer B; 41 min, 60% buffer B; 43 min, 80% buffer B; 49 min, 80% buffer B; 50 min, 2% buffer B. The TSQ Quantiva was operated in SRM mode with the following parameters: cycle time, 1.5 s; spray voltage, 1800 V; collision gas pressure, 2 mTorr; Q1 and Q3 resolution, 0.7 FWHM; ion transfer tube temperature 300° C. SRM transitions were acquired for the HTRA1 peptide “LHRPPVIVLQR” and an isotope labelled (L-[U-13C, U-15N]R) synthetic version, which was used an internal standard.

Data analysis was performed using Skyline version 3.6.

### Western Blot

Dissected retina sample in 0.5 Precellyses tubes (CK14_0.5 ml, Bertin Technologies) were lysed and homogenized in RIPA lysis buffer (20-188, Milipore) with protease inhibitors (Complete EDTA-free Proteases-Inhibitor Mini, 11 836 170 001, Roche).

Vitreous sample were added to a 0.5 Precellyses tubes (CK14_0.5 ml, Bertin Technologies) were lysed and homogenized in ¼×RIPA lysis buffer (20-188, Milipore) with protease inhibitors (Complete EDTA-free Proteases-Inhibitor Mini, 11 836 170 001, Roche).

Samples (retina 20 μg protein, vitreous 40 μg protein) were analyzed on 4-15% gradient gel (#567-8084 Bio-Rad) under reducing conditions and transferred on Nitrocellulose (#170-4159 Bio-Rad) using a Trans-Blot Turbo Device from Bio-Rad.

Primary antibodies: Rabbit anti human HTRA1 (SF1) was a kind gift of Sascha Fauser (University of Cologne), mouse anti human Gapdh (#98795 Sigma-Aldrich). Secondary antibody: goat anti rabbit 800CW and goat anti mouse 680RD were from Li-Cor Blot was imaged and analyzed on an Odyssee CLX from Li-Cor.

### Example 10—Cynomolgus Monkey In Vivo Assessment: HTRA1 Protein Determination in Aqueous Humor and Comparison to HTRA1 mRNA and Protein Inhibition in Retina

Experimental Methodology: See above example. Aqueous humor samples were taken and samples were prepared as according to example 9 vitreous humor samples. Cynomolgus Monkey Aqueous humor samples (AH) were analyzed with a size-based assay on a Analytical Methodology: Capillary Electrophoresis System (Peggy Sue™, Proteinsimple)

Samples were thawed on ice and used undiluted. For quantification, recombinant HTRA1-S328A mutant (Origene #TP700208). Preparation was as described by the provider. Primary rabbit anti-human HTRA Antibody SF1 was provided by Prof. Dr. Sascha Fauser and used diluted 1:300. All other reagents were from Proteinsimple.

Samples were processed in technical triplicate, calibration curve in duplicate using a 12-230 kDa Separation module. Area under the peak was computed and analyzed using Xlfit (IDBS software).

### Results

| FIG. | Compound | | | |
|---|---|---|---|---|
| numbering | ID | mRNA_retina | protein_retina | protein_AH |
| PBS | — | 82 | 101 | 95 |
| PBS | — | 107 | 99 | 118 |
| # 15.3 | B | 56 | 73 | 51 |
| # 15.3 | B | 52 | 53 | 68 |
| # 17 | # 73.1 | 23 | 41 | 47 |
| # 17 | # 73.1 | 26 | 44 | 44 |
| # 18 | # 86.1 | 32 | 29 | 44 |
| # 18 | # 86.1 | 23 | 28 | 64 |
| # 19 | # 67.1 | 34 | 39 | 44 |
| # 19 | # 67.1 | 34 | 61 | 42 |

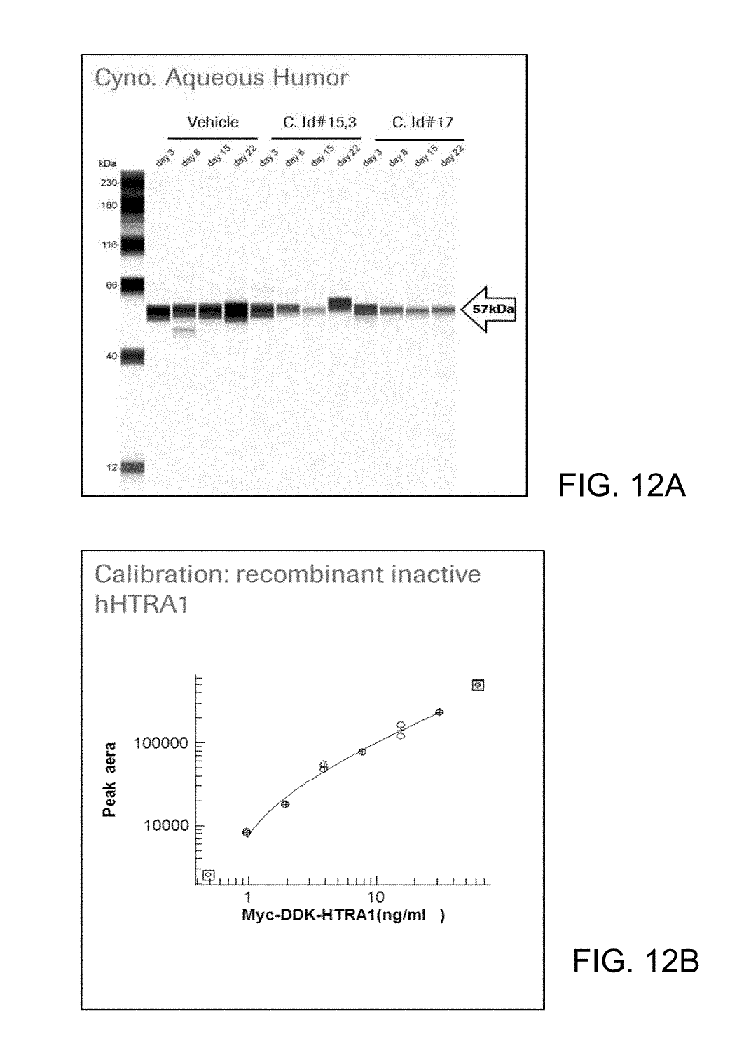

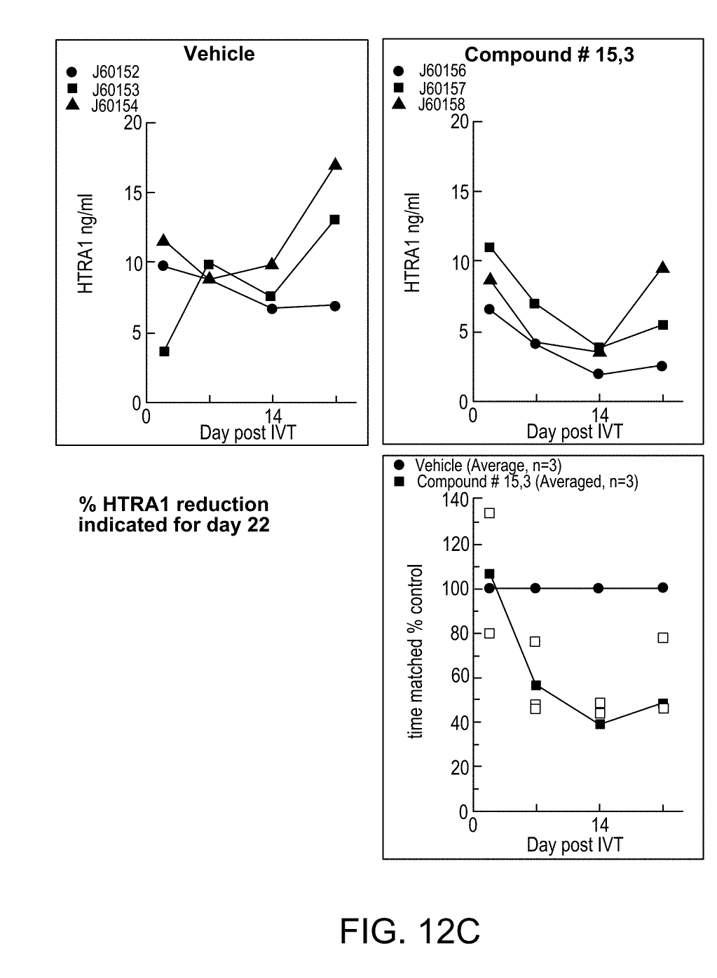

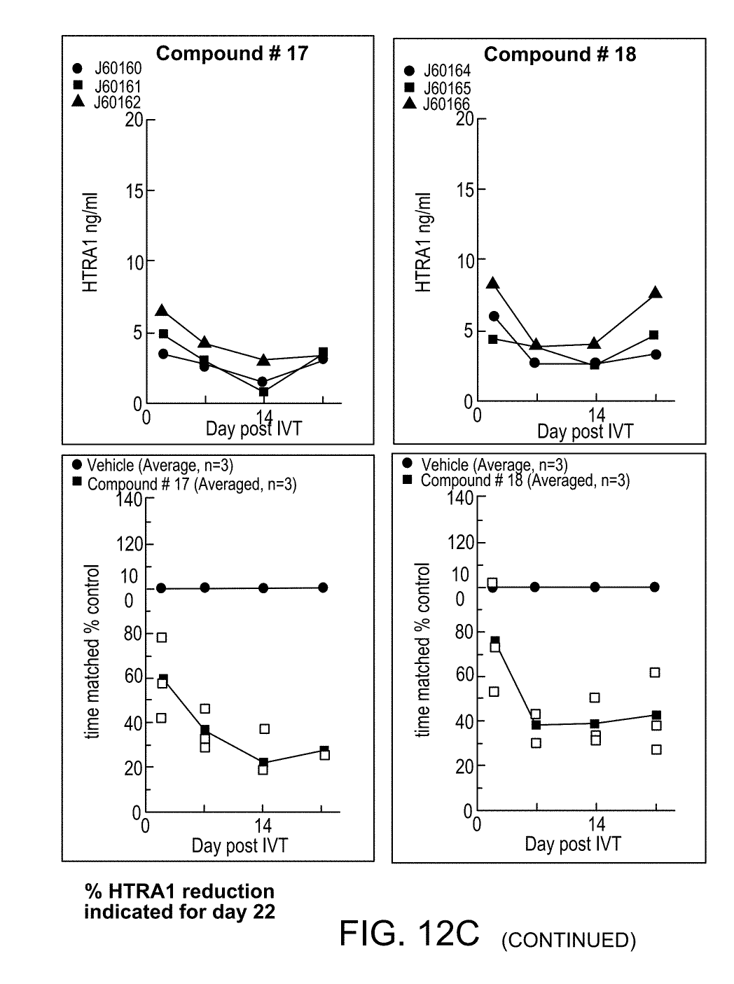

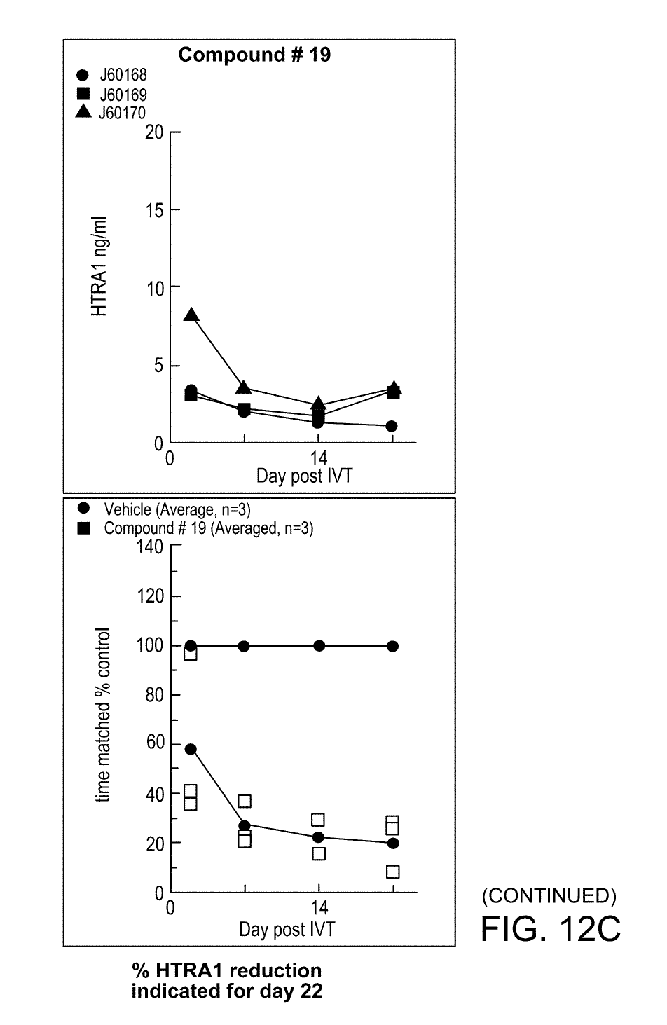

Note
the compound IDs shown in FIGS. 12-14 utilize a different numbering system as the rest of the examples. The above table provides the key to the numbering used FIGS. 12-14 as compared to that used in the previous examples and elsewhere herein.

FIG. 12A shows a visualization of the HTRA1 protein levels in the aqueous humor of monkeys administered with compounds B and #73,1, with samples taken at days 3, 8, 15, and 22 post-injection. FIG. 12B provides the calibration curve used in calculating HTRA1 protein levels.

FIG. 12C provides the calculated HTRA1 levels from aqueous humor from individual animal was plotted against time post injection.

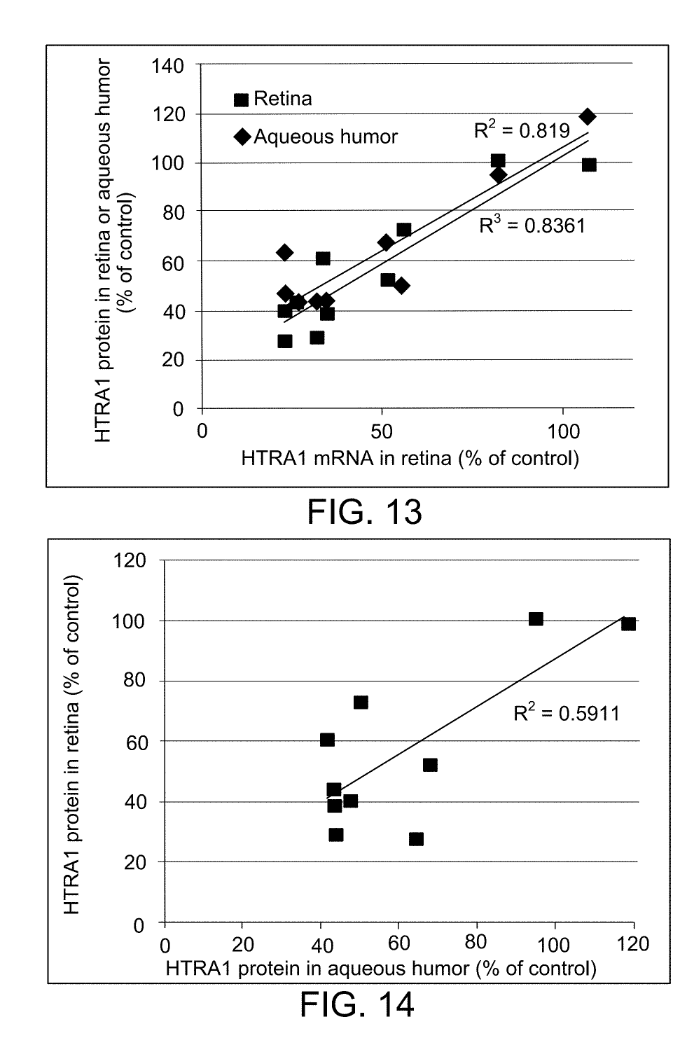

FIG. 13 illustrates a direct correlation between the level of HTRA1 protein in the aqueous humor and the level of HTRA1 mRNA in the retina. Aqueous humor HTRA1 protein levels may therefore be used as a biomarker for HTRA1 retina mRNA levels or HTRA1 retinal mRNA inhibition.

FIG. 14 illustrates that there is also a correlation between HTRA1 protein levels in retina and the HTRA1 protein levels in aqueous humor, although the correlation was not, in this experiment, as strong as the correlation between HTRA1 mRNA inhibition in the retina and HTRA1 protein levels in the aqueous humor, indicating that aqueous humor HTRA1 protein levels are particularly suited as biomarker for HTRA1 mRNA antagonists.

1. An antisense oligonucleotide of 10-30 nucleotides in length, wherein said antisense oligonucleotide targets a HTRA1 nucleic acid, and comprises a contiguous nucleotide region of 10-22 nucleotides which are at least 90% such as 100% complementarity to SEQ ID NO 113.

2. The antisense oligonucleotide according to claim 1, wherein the contiguous nucleotide region is fully complementary to a sequence selected from the group consisting of SEQ ID No 231, 186, 192 and 205.

3. The antisense oligonucleotide according to claim 1 or 2, wherein the contiguous nucleotide region comprises at least 12 contiguous nucleotides fully complementary to a sequence selected from the group consisting of SEQ ID NO 124-230.

4. The antisense oligonucleotide according to any one of claims 1-3, wherein the contiguous nucleotide region comprises at least 12 contiguous nucleotides which are fully complementary to SEQ ID NO 113.

5. The antisense oligonucleotide according to any one of claims 1-4, wherein the contiguous nucleotide region comprises at least 14 contiguous nucleotides which are fully complementary to SEQ ID NO 113.

6. The antisense oligonucleotide according to any one of claims 1-5, wherein the contiguous nucleotide region of the oligonucleotide consists or comprises of a sequence selected from any one of SEQ ID NO 67, 73 and 86, or at least 12 contiguous nucleotides thereof.

7. The antisense oligonucleotide according to any one of claims 1-6 wherein the contiguous nucleotide region of the oligonucleotide comprises one or more 2′ sugar modified nucleosides such as one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

8. The antisense oligonucleotide according to any one of claims 1-7, where the contiguous nucleotide region of the oligonucleotide comprises at least one modified internucleoside linkage, such as one or more phosphorothioate internucleoside linkages, or such as all the internucleoside linkages within the contiguous nucleotide region are phosphorothioate internucleoside linkages.

9. The antisense oligonucleotide according to any one of claims 1-8, wherein the oligonucleotide or contiguous nucleotide sequence thereof is or comprises a gapmer such as a gapmer of formula 5′-F-G-F′-3′, where region F and F′ independently comprise 1-7 sugar modified nucleosides and G is a region 6-16 nucleosides which is capable of recruiting RNaseH, wherein the nucleosides of regions F and F′ which are adjacent to region G are sugar modified nucleosides.

10. The antisense oligonucleotide according to claim 9, wherein at least one of or both of region F and F′ each comprise at least one LNA nucleoside.

11. The antisense oligonucleotide according to any one of claims 1-10, wherein the contiguous nucleotide region is selected from the group selected from:
(SEQ ID NO 67)
TTCtatctacgcaTTG,
(SEQ ID NO 73)
CTTCttctatctacgcAT,
and
(SEQ ID NO 86)
TACTttaatagcTCAA;
wherein capital letters are LNA nucleotides, and lower case letters are DNA nucleosides, and cytosine residues are optionally 5-methyl cytosine.

12. The antisense oligonucleotide according to claim 10 or 11, wherein the LNA nucleosides are beta-D-oxy LNA nucleosides.

13. The antisense oligonucleotide according to any one of claims 1-12, wherein the internucleoside linkages between the nucleotides of the contiguous nucleotide region are all phosphorothioate internucleotides linkages.

14. An oligonucleotide comprising or consisting of an oligonucleotide selected from the group consisting of:
(SEQ ID NO 67.1)
TₛTₛ^{m}Cₛtₛaₛtₛcₛtₛaₛ^{m}cₛgₛcₛaₛTₛTₛG,
(SEQ ID NO 73.1)
^{m}CₛTₛTₛ^{m}Cₛtₛtₛcₛtₛaₛtₛcₛtₛaₛ^{m}cₛgₛcₛAₛT,
and
(SEQ ID NO 86.1)
TₛAₛ^{m}CₛTₛtₛtₛaₛaₛtₛaₛgₛcₛTₛ^{m}CₛAₛA;
wherein capital letters represent beta-D-oxy LNA nucleosides, lower case letters are DNA nucleosides, subscript s represents a phosphorothioate internucleoside linkage, and ^{m}C represent 5 methyl cytosine beta-D-oxy LNA nucleosides, and ^{m}c represents 5 methyl cytosine DNA nucleosides.

15. A pharmaceutically acceptable salt of any one of the oligonucleotides of claims 1-14.

16. A conjugate comprising the oligonucleotide according to any one of claims 1-15, and at least one conjugate moiety covalently attached to said oligonucleotide.

17. A pharmaceutical composition comprising the oligonucleotide of claim 1-15 or the conjugate of claim 16 and a pharmaceutically acceptable diluent, solvent, carrier, salt and/or adjuvant.

18. An in vivo or in vitro method for modulating HTRA1 expression in a target cell which is expressing HTRA1, said method comprising administering an oligonucleotide of any one of claims 1-15 or the conjugate according to claim 16 or the pharmaceutical composition of claim 17 in an effective amount to said cell.

19. A method for treating or preventing a disease comprising administering a therapeutically or prophylactically effective amount of an oligonucleotide of any one of claim 1-15 or the conjugate according to claim 16 or the pharmaceutical composition of claim 17 to a subject suffering from or susceptible to the disease.

20. The oligonucleotide of any one of claims 1-15 or the conjugate according to claim 16 or the pharmaceutical composition of claim 17 for use in medicine.

21. The oligonucleotide of any one of claims 1-15 or the conjugate according to claim 16 or the pharmaceutical composition of claim 17 for use in the treatment or prevention of a disease is selected from the group consisting of macular degeneration (such as wetAMD, dryAMD, geographic atrophy, intermediate dAMD, diabetic retinopathy), Parkinson's disease, Alzhiemer's disease, Duchenne muscular dystrophy, arthritis, such as osteoarthritis, and familial ischemic cerebral small-vessel disease.

22. Use of the oligonucleotide of claim 1-15 or the conjugate according to claim 16 or the pharmaceutical composition of claim 17, for the preparation of a medicament for treatment or prevention of a disease is selected from the group consisting of macular degeneration (such as wetAMD, dryAMD, geographic atrophy, intermediate dAMD, diabetic retinopathy), Parkinson's disease, Alzhiemer's disease, Duchenne muscular dystrophy, arthritis, such as osteoarthritis, and familial ischemic cerebral small-vessel disease.

23. The use or method according to any one of claims 19-22 wherein the method or use is for the treatment of macular degeneration.


SEQUENCE LISTING (231 sequences)

SEQ ID NO: 1 - DNA, 2138 bp, homo sapiens
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgcaaaaaaaaaaaaaaaaaa

SEQ ID NO: 2 - DNA, 53384 bp, homo sapiens
caatgggctgggccgcgcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaaggtactccgccgcgctcctgggcagctccccactctctccatcccagctcggacctgcttctgcgggactggtgggcaggttgaggggcagcgaagcgttgtggggtggccagggcaactctcggggacaggcaggtgggccccggggtggcggatttccgcgggctgcctcggaaccgagcttcgcgcccagcccggggccggttctgcgcccagacgatgccagtacgcccggcctgcactctggggctcgagacgccgggcgaccggccatggagtgccctgagggcaaccacacagcgcggggaccccaggacaaataagaggaatgggggcataaaggaaggagagaagttcaggactgggaattggcgcctcgcagagcggcttcaggaccacaagaagtcatttcggttgctttttcttctatttacgtcctccgtcccctttaaaattcactgctttgatcacgggaccgctcagtgaaaactgtatgtaactcttttggaaaggaacagtgtttgccggcccgccccggagtttctccaaaaagtctaccccgagcagggaacggtttggcaccgctctcgtttcggcggcgttgctgcctgtcttgctttcctcgttttgagccagccctacaaaaatgaaagtggctccttttgaataagctgaatcgggctttggatcacgaaatctgcagaggcggagaagggaccgggttagtgatgaggaagaagtctacccctctgttcctacagccgcacacaggacctgttctggcaggggagacggtggtgatgggggaaggagtggaatggagcaatgtctaactctctcgcgggaccttccggagagatgctcctcatcttcaggcagaggccatgtggaaaaataatatcgagttcagcagcggccagccccgcgttgtaggaaccagacagcggggcttggcagtgcgcttgggcgcagccgtgccgctgctgccggaccccagtgctgcctcctcaacacgggcagtgccaggagaggggcataggggagcacagtgcagagggactggtctagagtttactttataggaatatggttcggtgtgaccaactagggcttagcatagtttggcttacgtggacgggaagatgccagagccgaactgggtgaaattcgagattgcgtatttcaccaacacaggagcacagccctcgggaaactcagcctagtcaggcagtagagagttgtcccggagagaagtgatcctgcagactcgagaaggggcatgatgatagcacacgtctgttgagcacccagtctgtgtgccgggtgtgttacctctgtgacctcatttggtcaaacgaggaggcagttgctcctctctctctctttttttttcttaagagacagggtctccctctgtcgcccatgctggagtgtagtggtgtgatcatggctcactgcagcctccgacccctgggctcaatgattctcctgcttcagcctcccaagtggctgggactacaggcggatgccaccacacccagcttctcattcccgttttacagatagcggagctaaggttgaaaaacttgcccaaggtcattcagctggaatttaaacccagacagcctcattcagaggagtcagcccagcacttaactccaagggtgtgggagaggggtcaggtgctgtaaatttcctggtgggctggacgtgcatccccctcagagctgggaacagcatacacaaagcctaagacttgtttggaggtgaatagatcagtgtggctggggaacgttttgggagggcagcaggagtgagccaggctggtggcccagagtcccagggctgaagaggctggctgtgccccgtgccctgtgcgcagatgttcttgaactggagcaactcaaagcctagtgtagtgtagggctgacctagcagtggagtgcggaatgcatccagggtggagagtttagactactgcaataatctgggtgtgaggcgacaacattgaaaaagcatgtttttgtccaaaacaagccagctgttactggtctcgctgtttgtggtctcatcgcacggggtcctgagttgctggcaccatgcgagccgcctaatttattgctagtgaggcaagttgcttaacaagttttggagttggctgagtccctgtgtggaggaaaacaggtcccccattggccatcgggctcacagcgggcccccggtgtaccagtgaggggacagccacagagggataagcatggtggctttgaaaggagggagagacagagtgggtacaatgcttttcttatccctccctccttcttttgcaaatatttattgagctctgtagggtgtctgacaccgtttgcatgtttgtctgtctggcacatcggaggtacttggtacgagtggattagtgaatgaataaatgaatgaatgaagacaaacgggaggtgcttgcgatacacagccattctgtttttccttagtggaaggcactgctttgctgcgccccctctctggatctcacactccacccttgacttttcggaggtgtttccgaggacaggcgcctgggagccagcagacttcattcagtccaagccaggctccaggactcaacagctggtgcccacgggcaggtcacttgacgtcactgttaaatgaggtgaattggctgcctgctctggctggaagattggcgggagagtcactttagctgccatggacatgagccttttctaggggtgccacttgactagaggcctggagttggagcaagtcatacacggatctggagacagagctctcgaggcaggagcgggtgctgcgatttcaaatattataaggtggctttgtctggggcagagcatgccaggggatgagaggtagaaatgtcatcagatcaggggtccccagggaggtgactagcactttgggtcacagtagatctttggatagaggaacatgtcaccattcaaaggaaagcactttcatctgtaagctgtttattgaatagacctcagagaacatctctgctcaccgctctggaaatgaaggcaaatcatctatttcagaagtcaatgcactggcagggtttggatgggaaagtatacaattcagctagagaacaaagatctgtcatctccagctgtactggtcagatgattacaaaaaagaaaggaattgaaatactaatagggtactaataatgagggctaacatatatgttgtgcttattctatgccgggtgcatactaattcatttgatcctccggacagtcctatgagtgagtgctgtagtcttccctgggttacagctgggcagctaagtcacagagaagtaccttgctcaggactggtggtcccacacaactggatggagagcctcgttcataaccaccatgctgtgctgttgacagagcaacagagattttaaaccaaccccagctaagccccagctaatagctgaaataaacagggctccagatggctgtggcttagagatggaacaggacagatcacagccttcactctgcaggctcaggagcctgaagacaaggttgcctccagttgccgtcagtgcagccctcactaaagaaaagcaaaaagagccgagggactgtaggaaggctgtttccaagccagagatccagacaaactgctcttgaagagagaaagcccttccagattcccccatgtcccaaaagaccagccgggattccggacctctgctaaaacatggacaagaagccaggaacgagacctgaaacagacttcccaaacagcagaagcctcatccatttctcctgctagtacatcctccaggaaagcccaccctactccatgcagcagcccagacaagcttggaggtctgcaagctgcaggggtgcccagaaactccacccctggaggtttttaggatcgcctgctcctggtctcaccccagagcctctaaaggcagaggctgtatgtacatacctggtgaagaaccaagggcttagatggttgctttacttcttggagccctggaatgtttgtaaaatttacttttttttttgagacagtgtctcgttctgtcgcctacgctggagtgcagtggcgcgatctcggctcactgcaagctccacctcccgggttcatgccattctcctgccttagcctccagagtagctgggactacaggcacccgccaccacgcccagctaatttttttgtattttttggtagagacggggtttcaccgtgttagccaggatggtctccatctcctgaccttgtgatccgcccgctttggcctcccaaagtgctgggattacaggagtgagccactgcacccgtgccaaaatgtactttatttaggtgactctttcgtgggaacctcaaacaagcaatcattgctagctgagtgctgaccctgtactgagctctggggagacagggttgaataaaacaaagtcactgcccacaggtaacttatattcaatacaatgggggaaaatacaatcactgcttccctggggttgtatttttccattgttaaagtgggcagtttgctcgagagtcattttcactattggcaattcaaatacaccttttgtcagttaaaaaacaagtgtgccagggacctgagcttcatcttagggcagggtgggtggaaacatttgtgagtctccagcttttagtcacctgaaacttggaaacttggaggtcttttgagcagtttatgagtctctgcctgctctggtcggctgccttcttttattgctctgttggttttgctaaagagttaaaatattaaggcttcataaaattaggaagttaacaagctcaaaaaccaagtgtttgagttacttcattccactgagagagctgtaaatgggttgcattggaacttaaaataactgcattgagtaagtgatggtggcgggcaccatgagctaactgtggtcagaagcctgatggcctccgctttggggctggattctccgtttggagctgtgtgatcctggatgagtttcatgccttggattcagaaatcagactttccatgagcttatatttcaagtgaataaatagctctggtcaggcttaatttgaagaagaagtaagcttggcagtgggtgagggttccttggaaggccaactggggcggaggggctgagggcaagcggctctggcccttcctggggtgttacctgaccaggtaacagctccctcgacctctcggagcctcggcagtgaggggattgggccagttgatctctgaggctccttttaactagaatggtctgggatttttctaagaaaacaagtctttgaggaggttgtggtcacctcattcctaatttaaagcctggggaggcttccttatgagctacttctttttcctaaattattgatggttaaagccaaggctggcatcgaatagatgtgatccatcttgagcctggttgctttgtgtttcagctttgtactggctgctgaagtccccgggagaccacaggggtgacatgttcatctccaagagatgagcttccacgagactcataccccttgctccttccctggggctccaaggcctttgggtcatctgaagtgagatacccttgtgtcatttcatcttttccttctccaccttctctgccgttaaaaaaaaaagaagaaagagaaaaatcctattaatagagaaaccgagaagtgtagccattctgaatgtgtttccaaaaggctcctggaagtggcatggaagttacagtgattcagcactacttggtgacgtgtgcctagaaccacagggggacattagccaggacaacacgcctcaggacagaagtaagtggctgcgaagaggcatgtccatcactgccggaaagatgcagagttcagtttttggagtcagtgctgagagttccatttctaaattcattcagagcatttatttaacacctactgtgtgctcagaagtgtatcaggtatggggactcagaggtaagggctggtggcccctgatctcaaggtactcgtggtagatagtatgatgctcagcttaagggctgggcttctgaagtcggattgccattttctggatgtgtggtgtttcttgggtgacttcatctctaagtctcagtttccccatcagtaagataagagaagtaatagcagatacatacgtagctcttagggcattgcagaatggaaggacctccttatatgaaacgcaaagcactgtgcctgatgcattgctagaactcaggcaatattagcgtgttgtcattgtcatcatcatcatcatcatcatcatcatcatcatcatcatcttcaaggcactgacaaaggagtcagctgtgtgggaggagtgctgggacactcttgtctccctggggatgaggtgggtgggtgggttaggaaatcttcacagagaaggagggtgatgtgagacttctgtccgggagctgactcggaatttgccatctaatatgttggaaaaggttctctgggcagaggtatccaaagtcactttgcctgtcaccctttgaggtcccagttgttgcctatatcatgtgaccagtgtgtggcttctcttgaattaagagctgcatgtctggactgcctgggattttacagatgtcatctcgttaactctccctggagcttgtgacacccaggagatggcagtttatagaagccctggcaccttcttgaatgatgcttggtttggtttctatgcactgggaattcctcacaaggaaagatttgtcacatcttaaggaaggaaaaaaaggcaaatttgggagtccatggataccctattattttagattccaggacaaattgtcgaataagcacgtttcataaaaacaatcctccgcagcatcccgtgacagcagctggtccctcgccacaggataattatgtctccttgtgcacacaaaagtctccgagggcatattgttgtggctggagtttctgataatttccaaattgaacaacctcagtcctaatgagtcagaggcttgtgcaatattttcaaacctcaggaacatctttttcattagttgtgcaataaagatggtaggcctatctctgtgatgagctgtttttttttctcaaagtttgatgagattcgccgtagaattccttctcacatagtcttgggcaagattttacccgatcttccaacacatgagtcatctcatatcctgtgactaagaagagctgtctctttggtgccagttttctaagtgcagtcaccacttgatggagacggatggacacagttgggattgcccaggcagatgggcaatcttgccagctagacataggggagggaagcctcaatgttcagcggtcacatctgcttttctgtggcacagagtgagctatacaggaatattgtattctccaggacagttagggcagtgggaaatgtcatcaaacagaacagtgacccaaagagccactgccactgggtgctctgtgggagctgggcactgtgctcattgtgttatgggccttgctttgttcttaccttgtagccacccagagaggcagggcattatccttgcttcctagctgaggccacagaagaggctcctagaggttagctgtaacttgtccaaggccagccagtgcaaggaggcagagccaggatttgagcccatgtctgtttcactcccaaactattcttcagatttctttaagtcaagtgttatttagaaatgttttgtttattcatcaaatatttggtgggtgtttccagctatctttctgttattaatttctagtttaattctattgtgggctgagaatatattttgtatgatttctattctattacgtttgttagggtgtattttctggtctagaatgtggtctgtcttggtgagtgttccctgtgtgcttgagaggaatgtgtgttctgtcattgttgaatggagtgttctataaatgtcacttaggtctagtggattgatagtgcggttcaggtcaactgtatccttcctgattttctgcctactgatctatcaattcctgaaagagaagtgttgacgtctcctgagtctattctgaaacactgaattgcggtctccatgatgaaccactagagttagaaaacctgggtcctagccccatttgggcctttgggatgactcccttctgcctcagtttcctcatctacaacagggggacaatgatgctgcctaggagacatcagcaggatactgtgaaagtccagtggcataaggggtatggaggagcttcgtcaactcctaaagcttcagtgctaggaatcctaaagcattgaaatccaaagatataaggaatatgaaggagttttgtcaattcctaatgcttcagtgctaggaatcctaaagcattaaagtccaatgatataaggaatatgaaggagctttgtcaactcctaaagcttcaatgctaggaatcctaaagcattgaagtccagtgatataaggaatatgaaggagttttatcaactcccaatgcttcagtgctaggaatcctaaagcactgaagtccaatgatacaaggaatatgaaggagctttgtcaactcctaaagcttcagtgctttaggagtcctaaagcattgaagctgtaagagattaggacctctagttggcaattccagactcttccaggactcctgatagagccaacaccaagaatagtgaagccagaaggatggaaatagtaaaatgcctcctgggtgtcaaagcatgggtctcctctgggcatgttctcttgtcctactgagacatgatagctcttggccaaagtgactgaacttgaccctctgtttcaggaaggccaaatgcagggttcactaccatcatgtccaagggcagatgcgttggtccagaacatcagcatcccaatcattataccaagcaaacagccgtctctgcctgcaccgtggagagcacacgctcctcctggggtggcctgcatcctgtgttcttctcaggccgactttctgtttaatgtttgctggtcaggaaatggcctgagctgaggttcttcagatcccagtctgacctttctccaccagcatttgtggctctgaaaaatatagcccagtgtggtttagccccactggatgaaacccagtaggaaaagtctgataatagcagaagacgcacaggaggaagagtgaggatttgagagcatctgggaaggaccatgtgcctggatatcgttctgtctgtgggattctgtgacacttgtcatttacagtctgttcccatggaattctcatcattggccaaacatatagtccttctgtcctctgaaaaatatcattctgctccgacctttcacacccatctctgaccacatcaactccctgtttgcatgcatcttgtggatgaaggacaccactttacctgtaaagacactggtggcttcccaaagccaccaactgacttgtagagaagacagaatcccagagtatgaaacctgagggtgaagggtcctggcaggtcctagagctcaacccttcacttcacaggtggggaaactgagggagccaatgggaacatgactctcacaagctgcacagctcatctgtaggggccagtgtggagtctgtttgtcctgagacccagggctgagcctttgagccctccgcatctcagccgcatcctcctgttggagcagttaggtgtttgggagaggccacggtccatgctcatggttttcctgtaaggctggagaaacaggccttgttcccttagtctctctaatcaaaatgaggttgcagaaaacccttctccctacttctccctaaaataatttccttgggttagaagatgactaaaagactattcatccgatgactgatgtctcccttcaagagttataagcacatataaatgcctttgaatggtaattataataattttgctgaagggaaaatatcagtataaatatcatggtggacacatggaatgaggactgagatgctttcatgtcttttcagctgtggttagattttctttaagcagaatatacaagtttttcctctcctagcataaggactctttttttttgtatcttttctctctactttttagacatgatggaaaatgcatttatacatttgatgacatattgtactatctcagttgtttaaaattataaatgtaatttaatcatatgaaaaattaagaaaagaagattcatatttcaccatcatctccccagaaatatcatttctttattactattattattattattattattattattattattattattattttgagacagggtcttgctccatcacccaggctggagtaaggggcacgatcttgactccctgcaacctccacctcccaggttcaagcagttctcatgcctcagcctcctcagtagctgggattacaggcctgcaccaccacacccagctaccttttatatttttaagtagagacagtttcgccatgttggccagactggtctcgaactcctggcctcaagtgattggcctgcttcagcctcccaaagtgtggggattacaggcatgagctaccatgcctggcctaattccatcatttctgtcccaagtgttgccaccgtttggttaactgttcccctgttcacatccatttgggccaaggttgcaatgttaaacaatcctgagatggacattttcatgtttatggctatttctgtatctagggtcattctcttaggagaggtactaaggagtacaaaaactgggaagaaggatatggaatttttatggatctggtataaattgccaaattattttccagaagggttgtagccatatttgttgccatcagctctagaatttcaacctcgtaagtcactgaaagaaattctcccaaaatcaatccttcaggaataatggaagaagatggtgccaaaccccagccattctgctcactgttagattccttttttggtcttacaggttacttttattctcaggttgatggctcttagagttgagcaatgtttggggtagaataacgagcacttttaaaacttggttctacctggggagggggtgagttgtgatcacagacagtctcacctgggaggggcttgggtgtttgtcggcttgtccttctaacactcgtgtctcaggcgagcagcctgggaccagtgaggtgacctgaaggctggaggtcacaagctaagaggcgacagagaacccaggtctcaggaagcccagcccagagctcgctgcactgagcctctcggatgccagctctgtccaggatgcgggaggaggccagactgatttggtctgttttgaaaagtgatgaaaatatttattcaaatgttttgtactcataggcagaagtataacaggagctgcatatacaaaattattttctagtagtcacattaaaaaagtaaaaagaaagaacacgattatttttctttttaaaacagctttattgagagataatttacatactataaaatttacccctttaaagtgtacaatttgctgttcttatatattcacaatcatgcacgtatcactaccagctccaggacactttcatcaccgtaaaaagaaaccccgtatccattagtagccaccccatacttctcctctgcccagccctaggaaaccaccggttcattttctatttctatgaatttgcttattctggacatttcatataaatggaatcaaagaatacgtaacgggcttctgtctcttagcataatgttttcaaggttgtccacattgtagcatggatcattatttcattccattttatgattaaaaatatgccttttaagggatacagggagaccagacgtctattttatctcccctccctgatggggaatcctaatttcagcctggaaagtcactgcgaaagtctaaactgcagaggtgatactgtttccactggaagaaactgtagcacctgactcaggaagccagcattaaaaccaagaatattctatatggatggggattacgcactgaaaggaaaacatgaggaaatgcacttttcagatttattagatcatagaacttttttggagctggaaaggatgtcggaaaccgtctagcctaccccctcatcttaccactgaggtaactgaggcccaggaaggggaagtggcttgttttgggtccgggaccactcttcatttcttatttgagccaaagcttccttctggcgtctgtctctgtttcacaagttcccctcgcatgggggctgggtactgcttggaagaactggcttcttccttgatacaggggctcgttcaccatcacctccctccctcacgtctcttctgcctctctgcagcctcaggccctcctcctgcaccaggggggcagactcaacccgggtgggcactgcctcccagtccgtggccagaggctggagggctagggagactgaacagccccggcagctccagacataacaacctatgttgaggagtcagggcaggaagcgaacccagctgagaaatctgcgaaggtcaggaccagagccagacgcttatcaagagcaaagttaatggtttttgtgaaccgagcagtcagctgtttccccgaagataataatagacacatcatgttgggcattcaggaggcatctgaaaaaaaaaatgtgcagtggaattgattggaagcttttccctaatgcataaaataggccagaaaagactatcaaatgtaacagcaccgatcaaacccaatagatcaagcaaggactgaaaaacacaattttttttttctttgccagtgagtctgaaaagtgattttcaatgacaggcgcctttaaacatagacaacataaacaacaacatagttgttctggaagaggcatcttttcccagtaaagccaaagatgcagatctaggctgtgcttgtgactgacagcacagtgaggggttcacagccagctggccaggtgccccccgaaagcacatttcgaatctactctatttgagagagactgccttagccttgtttgggtaagtcttcctccttcacttcacctgccacagacttttccaggcaccatctgctgcagtcttggcccagcccctgcaacagttactgctcaaggcacccgggacatgcaggacgggggagcagcctgaggtctggcgtccggcgagcttttcccacttggagccgtctgggagactgtcccggaaagagaggggctgccaacacttggaagtgccaatgtgtgctgcaagtcgaggccaggctcccggctcccccgcctcttcctccttgattcattaaaaggaaagaaagaggccacacgaaactctcctgaatttcatttctttgtttctatgcaaaagacagagcgtggtcattcatcattcaaattttagcctttttaaacaaataataattcctgcttgtgaattcagtgtattttaacaagagtaggtctgagggccgttggccgtgtctttccttagatttgcagacagcggccctgatggtgcatagggtttcaggtttcctttagacctcagctggctgcctgggccaccacttagcaatgccattgtccttcctgtgcattttctttgcagaattcgaggaaatccagtcgcacaggcccctctgtgcccatgtccccggcgccctggaatgtgcagtaccagcagcagcgattagaatgggggtctggtttcccggaatgtgcaaggtctggcttctgtttctgctgcctccatgccccagaccagtgctgggccgggctctgggctggagccgtggctgacaagtttccttggaatttaatggagcgggccagacagcatgcagccactcaaactgaaaacctgggaaagaaatgagtgttgtggggcagctttgctgcattcactgggtcatatatgcttctttttcttttcctcaggcaacccctcttgcagacaggaggccccctcccctttcgcttcatgcctcactggccattaggaaccttttaaaactgatttctctcctgaccctcagagagaacatagtccaagttccctggaggaggaggaagcgctctgtgtttctctgcagttcacggctcagttaaatgcagcctacgtgctgtctttccccactcctctgcctgctcccgttgtgcttctcatgatcattctcaaattcagcgagaaacctcacaaagggagcttttcttagggaagagtcatccttggcctcccgaatgtggaccagcccctctccccagctgcacagcatcaggttagttaaccacctgcctccatctgggtcctgtctggacaggcctactcacacctgctgcaggcatccaacttgccctcaggtgcctgtggctcgtccagaggggtggagcccacattccagtcctgacaggtaaagttcagtggcggggaccctgcatttagtgtaaagatcaatattccaggtcctctcttcctgccacccagcgactggccgtttgcaggcactcggtcccagttgtcctgggcctgcagcccttgcattctctctgctttgtctctgctattgcacccctgccccatcagaaatgcaggtgggggggccttccgctgggacagtgagagactgggtagtaaggggagcgctagagggatggttgcgcttgcatccagccctgactgcattcgctctcccccgcctctctgtgaaggtgctgagctgtgagtggaaccaagtggatgagagtggccttgggcacctgccgataaatttcccggtgtgtcttctcctcctgggagtcccatctggatttgggtctggatttatttattcagcaagtagcctctttatagttactttttttttttttttttttttgagatggagtttcactttgtcacccaggctggagtgcactggcgcaatcttggctcactgcaagctccgccttccaggttcacgccattctcctgcctcagcctcccgagtagctgggaccacaggtgcctgccaccatgcctggctaattttttgtatttttagtagagactgggtttcactgtgttagccaggacggtctcgatctcctgacctcatgatctgcccaccttggcctcccaaagcgctgggattacaggtgtgagccaccatgcccggcctgtagttacttttaatttagccatgctcggggctgaaggggatgccaaagaaatataagatgagcccctcagacggctaaagatgaagatgaggcctccagtatgtacctcccacatacaccccaggaaattctgggtgtcactggattctggacctcccaaaagctgctggcacctggaggatggggccccgaggctggacctcactcctgctgggttgctggactgggaaagtactgatggcagctgaggagtgtgtcccagacttcactgagccattcccaaagattattccaagttctcctgacactgcactggaggcctgctgtgctggccttctttatttacagtttctgactggtgtctagcagccctgccagagagagcggcagtgtgtctgcaggcgaccaggagaaatgtctcaggctttagagcaggactttgagcacatagctgtgggggcccagcaggctgtctcctgcacggttacttctccttgtcctttcatggtcgagaggttgctgcctggcccttcaagtgaggatgggacatgctatccattggccttaatttccaacctctgcatgatgcattttatgctcctgcctttgaaagaacttttattttcttgtcatttatgcccagaccccacatggcagaaggaagggaggctgggacaggggaggcggataagctgccgctgacagacctgcccagtttcttagctcatcccggcctccatcctggtgagcagacactggcccaatccagccatatttttggctgagtttctgtcttcacatctcatccttaaccctgaatcctggccatagttggtactgggttgtattcttatttgtaatctttaaagtaggaatacctttgctggtatttaaagtggaagaaatcaggtgaagaatcacaagtgatttgcaaactggaagagacattagaatgtaaatgtgaggaagcgtcagcatgaggggcttgcctgggctgcacagcttgccttggctggagtatgcactgttctggcattgcagagaggatgggtaccttgcctccctgcaggtgggggactgtatcagcccccgcagactgctcctgggctcctgagtttgacagattttttttttttttttttgagacggactctcactctgttgcccaggctggagtgcagtggtgcgatctcggctcactgcaagctccacctcctgggttcacgccattctcctgcctcagcctcccgagtagctgggactacaggcgcctgccaccacgcctggctaattttttgtatttttagtagagacagggtttcaccgtgttagccaggatggtctcgatttcctgacctcatgatctgcctgccttggcctcccaaagtgctgggattacaggcttgagccactcgcccggctgagtttgaccagattaaggcagcatctccagtggcacctgagcagctcctgagatgcttttctgtgctaaatctggatttggggtattaaatcaaatgaatttgaaatgcaggcacagctggccccatgggcatggacctgtgcagtcacaccttgccccgtgttcagaagggtgctgtgcctgttttaatgctctgctgttgctctcttgagattcttaataatttttgaacaaagggccccacatactcattttgtactgggtactgcatattatgtagctagtcttgaatctaggacagtgcattaaaatgccattgattggatcaatctgctcttgcaactgatttgaattttgggaacatgctgtttcctgtgaataaaggaggattcatttcttttccctcgaatacactgcgttctgttttccaaattagctctacgtatcaactcagctgagaaattggaagcggggattgttctggctggaagggaaggttagattgttaatcctgcatcctggccctgatctcaccgagtgtgaagcatgttcccacaatggtgtgggctgcggggggctggaggctggctgagaaggtggggaccaaggagggaggctagcctgggagccagacagatggggttaggctcttgcttttgccactcgccagctctgaggcttagggcaacatgatttaattctctgatccttgtttttttcatctttctgtagactggtgatgagatgcaccctgcaggcttgcaggcttgcaggagtaattaaaggtaatatttgtgcctattattgggcttgacatatagtagatgctctacaataaatagatcctattattcttattgataatattattttattgctaacattgaaggttgggtgggatttgactagctggaggcgaggagaatgagatcatccaggccggaaggaaaagagacatgaatgcagggggatggggtggagcactttggaggtgtggggagaggtctgcagggtgggagttgtgcattaaggagtcgtggggagagtggaggaatcagtgccacatggtgaatgagaggggatcgtgggcccgaggagatggcgatggctgcggggatcctgcaggaagtttatgtgccccaaagtggcattatcagttagggggagacactgaagacagaggtgaggcctgcctgaattagcgtagagtgggattcttggaagcttcagaagcttgagaagagccacttggaggtgttgaaatgcacctgggagggacgtggggacccagctctgggctgagagctgggagacggaaacgcaggtgaccttggccttgaagatggggcatgatatttagtgctttatgtgcaatctcacctaggactcccaagccctttggagtaggtgatattagctccgtgttacagaaagggagactgaggctgaagcagggacattcatgatctgaagtcacacagctgtacggggcagaagtgggcatggaggcattaacttagagccgaaaggtgtgacctttcttagtgtggctggccccacggggaacgtgtgtgggttggagtacaacttggtgttcctacccatcccagatgctctgcgtttgtgaaccccagttgccacatcagggcgggcgagggcaggaagctctgcagggagaagggacaagggacagagccaagaacaggggcagtgccccagggtcctgcaggggcaatgaagggggttggcacacctgggttagttgctggccagtgtggggagagagctggcctgggagtctaatgggaatgccagggaaagctgccttggtcccctaaagtgaagcccccatgctggccatggagtgttggtgattgagggtccctgctagttgtctggccgaggcagcatgtcctataggcatagctctggtgtcctgctggcgtggcgtgagtgcccctcatgctgggagccagccctgtgctctggagggaggtggtgggaggacaagggacagtgggacctgccacctgagcaggaattggcaccttctcccactggcaggtccaggttttatggaatctgaaacttgtacaattcagtataccctcttcaagaaaaacacccctcaaaattatgaatataacattaggtatgaaactattattgatatagattgaaaaaagaaaatgcccaaaatgacaaacttcagaaaatagacaaatactgcaaacatcacaaaatcagaaaaataagattaaaaaaagctaactgctgaacactccgtcatcttgaaaatgcccctctctcctcctctattttttggctgtgaactctttgctcaccttttcatgtgacaatgcttttgtaatatttcctacagagaaaatagaataatttattattacttttattgtttttggattattattatgatcaattcaatatttttctgctacccacacactcactgtcttctgtccaacctctggcctgcaccaggggaaccagcagtttcccctgccatagggtgtccctggagaccacacatatagcaggatagatatagcaatttaactagacacagaagggacttcaaagccacaaatatatctcatttaacctgaacaaaatgattatccagttttacttttcccttagcctcttcccccaaatgctggcagccaccctgatgggatagatgtgtgacagagggcaagagaccgtggccccaaccagctgcagcttcactctttcatttctgtatactctctacaagctgtgatgatagcactttgctagggcccctcacagggcagatggagggctccacgctgaagctttgtggatgtttgctgtctatccacctctgctccttgtgcctatgcagggattcaggcccaaccactgcagagagcccaagagcatcaggcagaggttcccaaactgtcatgattggtggcacctttagtagttgatacggtttggttgtgtcctcacccaaatctcatcttgaattcccacatgttgtgggagggacccggttggtggtaattgaatcatgggggcagatctttcccgcactgttctcatgatagtgaataagtctcccaagatctgttggctttataaaggggagtttccctgcacaagctctctctctgactgctgccatccatgtaagacatgacatgctcctccttgcctcccaccatgattgtgaggcttccccagccacgtggaactggaagtccaataaaacctccttcttttgtaaatcacccagtctcaggtatgtctttatcagcagtgtgaaaatggactaatacagtagtgcagtcattttttcatggtccccagtaaggccaaaaaatacccaacagttccatttatcaattagtggaggccaaacaatttgataagtatttgtgtccctataacacagtggtcattaaaaaaagacattttaatttcattattcaataagcatgattacttatgaatgggatatgtgcacctgttgggtgtcacatgacctttcaaatcttggagtcagattggacaccaccatgcccatttccagttcaactctgatttttgtgtggtacatgctttttatcacagtgactgccagaaatccaacttcatatggaatcatgaaaagggatgtagtgtgatctgatttcaaaactatgatcaatctagagctagtttacaaggtgtctaacagtgatcaagtatcactgtatttccctagaaaacctgaaatatcgatgaattttctgtggcactctggggtcccttggggcacactatgggaaccatgggattaggaccataaggatatgattttggcttcttcctgcctcagatctaatctttacctggcatttttgccttaaagatgaaagaagcatacattttgatgtatttaaagcacatattcggccaggtgcggtggctcacacctgtagtcccagcactttgggtggctgaggcaggcagatcacaaggttggaagtttgagaccagcctgaccaacatggtgaaaccccatctctactaaaaatacaaaaaatagctgggtgtggtggcatgtgcctgtaatcccagctactcaggaggctgaggcaggagaatcacttgaacccaggaggcagaggttgcagtgaaccaagattgcaccactgcactccagcctgggctacagagcaagactctgtctcaaaaaaaaaaaaaaaaaaaaaaaagcacatattcattttgtgcttattcttttgagagaaacacagataaaagcctatcctttaattcatactccccatactgtgattttcatttttactgcaacaaattttgttcagtgtgataatgaatgtcaaacacttaatgccttgctcttttcagtaacatgacatattggagaataatgactgaagcttatctacactgcctacgtctgttttcttccaccttgaaagaagttgttgaaagtaattaagaagtattatgtgtaaaactccagggatgatgtgcttcaaggaagcaacatttatgaagttgtgtgcttgactagtagtttataaagaggaaagacgaatcatttattgtcttgggattgaatcttggcaatttttaaactataaagttacaggaaatgttggctgctcttaatgggccatttgttgtgttaaaaatcagtaatgagaaatatttactaggtaagtggaaagatccatctctataaattgttgtaacttaccattttacaaatcttagttactcagtttttctgcttaaaaatgaaatcatgtagcactgtataagtcattcagttttttattttggagaattactctggattgtctaggctctgtgctctccacatatatttttgaaatagtttgtgaatttctacaaaaactcctgctcagaattttcactgagagtatgcttaatctatgggttaatttgtgagaaattgatagcttaacaatagtgaatcttctgatctacaagtgtggtatttctctccatttatttaggtcttctttattttgatagcgttttgtagctttcaatgtacagatcttgcaaatatcttgttaaatatttccctaattacttgatatttatttttgatgctgttatagttatattttaaaaattttgattccaattgttgctaatacatagaaatgaaattatttattgacctcttatcctgtgacattgataaacgcagtcatatattcgtagatttctagaatttttctatatagactatcatatatatcatctgcaaataaagacggttttacattttcctttccaatctctatgccttttgtttctttctcatgcctcattgtgtggtccattactgaacggcagccagttccagctttctgttcaattaaggagcaggtaaaatggccaggccttgacctttcagggggcttcccgtcctcattgccttctgctgcctcagttctggcttaacagaacagtgtggggaggaggcatggtccttacctactagggcgttacttggccttcttcaggttggttgcttcgtcaggtttaagagctcacctgggctgcagttcaggctaggttatctgctgacctggccctgtctcccttctgtagtgtctgtggggtacccttgtaagctagggagaagagacacacgtgaaggccagaaaaaacagcctgccacacagcttccctggatcataccttcgcagtgacatgacgacgtcgttaggaggcgccgaggtggctgagtgggtctccagacacctccctttacctctctgctgtgccactgatgtgtgacttgcttacacctatgcagagctgccactgagcagcactgtggccagtcctttggattttcttctttctaaattgtatgccgtggcttgatcaagcatttcatatacagtagatcatgaaatcagcatagaaaacacattgaggtaggtggtgttaccacattttatggatgagaggctaacacttggaggagtcaggtaacatgtccaaggccacacagctagtgagtaccctgctgagggtcacactctggtccatctgaggccagagcctgtgccagccttctcctcatgctgatagacgaggaaacagaaagaaggagcagtggacgcccccaccctctgtcccctgaaccccttggagagtaggcagtggcagagccagcctgggcccatctatgggaattctccatcgggattgactcctctggaaggaagacagttgacccacagttgagatcacagcagatgggccagccagggtgtctgtagaccatcaggcagtggccactccatgtagtttaatggacaagcccttttaatggaacaggaatctaacactgaaccaagctgcttttagacacacttttattcctcactctgaaatggcgtttggacaagccaaatatttcttcttctttcagttgacattttgtccatctttgaactgttagttgatgcttcttctgtttagttattcctgttctattttcctgttgccactagtccacccagggatggtaagaatggaagtcaatggttgctttttcatctgagatgcaccacgaaggcttgtcagtcagccttgtcatatggtctgtgctcccactgctccttctttctgtttcctcatctgcagaatttggagagtcctggacctgatctcaaatttcacatgttatttatcttcctgcagcacgctggggagaggaagagacagggacatagaaggttggagctggaacagacttcacatctcattccagaggcatttggtccatcttacagatgaggaaatggaggctgctcagtggactgaggctggaactgggccttccagtggccaggccagatcctccttgatctcccttgttgctttcctggtgggaagaccctggaaccactttatgtgactgtgtgagaagggaactgcctctcattttacccagcaaaatccaccttcaatccatcttcatttttgcccctggtgtgggcaaattctcccatacctaattcaggaagccagaaagaggaagtgagttaatgatccttagtgggaaggcgctggtaatggtccttcttgtgagagtttctgaaacaccacgctgtctctgtgttctggcctggctggagttaaacctcttcttggcctttccccaggaagctggtctgaggaagcccagatgcgtttgtttacagctgtctggtgacattcgccaggctctgttttcagaaggaacatttccattcccttatttacacctcccattggagtgctcggggggacacaccaattatttgcaactacctggaaacctaggagggtagcagatctgtaggaggccagtgttgaagtgagaagctgtagatctggtgacactgtgggcttgggagggcttgcccagatctgttacttatactctctattaagaaacttcagtgtccatggagaagttatttaaagtctgcgagcctcagtttccccatatataatatgggaaggatacctgattttcctattccacatgaaggtagaaaaaattaaattaaggcagccaatgaaagggttttgaaagcaaaaataataatatgatactgttctgaatttgttaaattattcttccaagtagttgcagatctttttctgtaccttagaaaaaaaccatgctatgtaaaaggagatgattccaatctttaaataaagcaactcagaggtcaggggctaggacagaaaacggccctttgttcacagaagcgctctcacttccaagaaagcaagcgtgggagaggcaggtggtcctcccgatgtccctgtgccccatggtgtcaagctgggttactatggcccttcgtgacccagtgcagcagggatgtgggaaccagtgggtgtgaagctgtgacgggtcacaagagggctgggacgtctcacagcttttacttatagcctagagcctggggaagggttgccactctagtgatgagagaggcgtgtgtgtgtgtgtgtgtatgcgtctgtatgtatgtgtgcatttgcatgtatatatgtgtgactgtatgtatgtgcacatctgtgagtatatgaatgtgtgtggaagtgtgtataggtgtttatgtgacagtttgtgtgtaaatgtgggtgtatgtgtgggtgtgtttatgcatgtacatctgtgggtgtgtatgcatagtgtgtatgtgtgagtttgtgtgtgtgtgtgcatttgcatctctgtgtatatatgcatgtgtgttaggggcaggcacacaggcctgttggtaaatgagacacaaaatacctacaaaatacaaaatgtgagacaggaaatacaagccccagttactcatttttcagtgcaacagacataagattaccatgtgaaattgctatgaaagtttccgaaagcttcctgtcaattcgtagtgagcagctagcagaggagtgcgggtccctggagcctgcttgtgcaacgctgagctagtccaagggggaagaatggggtgcatggctctcagctgcagaccagcctggaacctctccagcctgctttagcagagacttgttaagaggtagcagcaggtggcaagattaggagccggagtagtaggctaaggctgcacttccagggacacactgcctctgccaccacccgtgccacgaaaatgggagcccaggaccctgaatctctagcagtccgtttctgaatcagttaccttgggtatgtgcctctggttgatggaaactaacttgtagccctgctgggtgagagcctcacatcgggacatgtgacagctttgttgaaagtagctttggaaacgcccaccacgtggggccactcactgtaatataaacggtcatgcatcactgagcaacagggatacgttctgagaaatgcgtcgttaggcgatttcatcactgtgggaatgttacagagtgtgcctacgcaaacctagatggcagagcccactccacacctaggccagatggcagagcctgttgtttctaggatgcacgcccgtacagtaggttactgtactgaatactgtaggcagttgtaacaatggtgagtatttgtgtattcaaacatagaaaaggtatagtaaaaacaatggtgttatggtccgcggctggctgaaacgttatgtggtgcatgactgtaggtataaagcattacagttgtttgatttttctctttttctcacccacagtcttaaggcacctcttatgccttttgtctgggatgtcccgggcagggttggaacgtgtggttaaggcatggcggaaactgctttggggacagacgatggcctcagcttgccttggggtgtcagtgggaaagataggagctgcccctttgccttcgtgtttcttcgtaataatctcagatgtacccgtctggtgggcctctcctagaaaaagccccggtgctctttgctcctgcggtgtttctcaggagggttgttgcttctttgtaatggtggggactcagggaagggacgcaggcagagggtgatgccacatcaaaaagggacccttggctgggtgtggtggcttacgcctgtaatcctagcactttgggaggccgaggcaggtggatcacctgaggtcaggagttcgagaccagcctggccaacgtggtgaaacccggtccctagtaaaaatacaaaaatacaaaggtggtgggtgcctgtaatcccagttactcagtaggctgaggcagaagaatcgcttgaaccggagaggtggaggttgtgatgagccaagattgcgccattgcactccagcctgggtgacagagtgcgactccatctaaaaataaactgaaaaaaaacaaaaaacaaacttgggccatcagcttcttggaaaggctggtgtgaggttgaagcatttgctggtgcctctgctcaacgtttttgtggtgaacctgagcaaagaggttatcattagtggattttactgccttacctgggtgggcactcccttgggaggtggatggacatttgcagctgagcccaggtgggggaattgcgctcactccgccttcagaattccaaaggctgggcatgcatcttggcttcctctaacccatgtctttctctaggtggccacagcagagtgtcattaagtatctattctttgcttttgttctcagggcaggaagatcccaacagtttgcgccataaatataactttatcgcggacgtggtggagaagatcgcccctgccgtggttcatatcgaattgtttcgcaagtaaagagagccttcctttttcctataacctccgaagctttcaccgccactagcaaaacatgagagctatttttgagatacattaaagtgtcaaagtgtcactgaatatcttcctacttaagataagtgtgtctcccttagaacattttccctattcgactatataaatctacattcttgacccttctgaatgtttaaagaacctcgggctctgaagagattctctaagaatattttgtaagtggaagtttttgatgcatgcaaaaaattggcaggatgtttagtgtttaaatgctaagcccgatatataaaggagcgatggctaggtgtgtgtggctgttgcacaacccattaatcaatgcgttgaagcgttcattttaaggtgctacaggcttaagtgtgtactcctttggattttaggcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccaggtaagggtgttctcgcctgcagaggtgagttctcagatgccccggaacacccttggcaaaggcaccagagctctctgattgcagctgattctcggggggcactgaagccagtctgagccagtcacaggagggccttgaggagatgctgagtatggcctgggggtgtgggagaggaaggggctcaggaaaacttctgtaaggagccagataaaagtttttaaaataatgttttaaatgtttgtcaaagaaagcaatagatttgtaaagaaattagtaggtaagtagtgaaaattgattctccttcccattcccaatcctgtggcaactcttgttacagattttatttatcctccacagatacatcatgcgttcacaatgaacatagaatttactgggttttagactgagccatccttaacttgtcaacagttactttgaaaacaaaccagctctcccaaattggggttttgcggggttatgagatgtgtttcaaaagaatgtttcgtactttaaacatcttggaaaacttgaattaaaacagagctaatggatttcttctttccagaccttctcagagcttttagtatgctagtgtgcacgtggcttgcctacaaaagggtgttgactgaactatttgcccaaattataatcatttgagtatacagctttttgtgggggcaggcagaactgagacataccaaaatcagtttgggaaatgctgtatttgaaaatgctttctatttaaatattctctttgcaatcatttttgctctgttgatttgcttagcaaagtcttcatgtctgggacaatatccatttcttactgactcatcaaaaacccccactcgacacgtcgatgagagaggttttgtttgctgtgtggcatgttcagtgaaagcgtggtttccagtttcttcacatccttataattttctagacttcagatggagggaacaatcagaggaggctggaatcctgcctctgaccaaggaaaagaccagaggctgagccaggtggggtctcttgtccagccctctgcttgcctcgctttacctgggtgtgggctgagtaattccagacaagcgtggaattaatctggctgtttgtgctgttcagtggcacgctggttacacctccttctggaaacaactctgcgtgtgctgtttgggtggtaggattccgggtctccttctccgtctttttataacatcaagttgctgcccagctcaggctcctttacggccagtcttcagaaaaccaccagctaacacatttactaccctccttccccgatgttcctgtagcttctctatggctgggtggccaggcatggccgaagaggctctgggtagatataggctctgtgcccggtgtgtgtaactggccttgagtgaggctgcagttgtgtgttatttctattaggtcactgtggaatttctagcgacaactaatctttcaaagtgtgtttattggtcacaggattattgggccagcctctgccttcattctttttcacctaatctgcataatagctgtgttatccccattttagagaagaagaaacaggggctcagagaagtctagtaacctgtgtgaggccacacagcaaacacctcatgaccctgccctcctaaggcagcccatggctactgctggagggatagaggccggccccgtggtttgatgggacagcttgaccttaaacagcccatgggaaggcgggtgcatctggtttaggaacaggctgctagaaaggtatccaggatgtggtagtctcaccggaaggagccagtcagaatagcacagcctgtggccacgcgtgggacctgttcagcctcatggagctttgggaggcagccagcagcagggcatgggctgtgtgcaggcgaggcgctggcctggacgccgcccccactgcgtaacttcgtgtttggaatgcgtgggcacataccgtgcggctgcttctggccgggggatattcttttccaattttgagccaaggtggagactgtctcctcgtgccatccctggcatgtcctggcaagacgtgaacgatctcaatagacgagctttgcagagtgtgtctgacctgactcctgctgtcttgggagtttagctcttcagccagcagcatgctgtttgacatgtgtttcaagccccccaagaaagggtgcttgaaatttaaaattgaactgatgtggcttttcaaaatggaattggaaatgaaaggatattaaattgcagacacccacacaaaagactggtttccactgactaaactgcttttttttgctgatagtagttgaaagtagggagagtaacagcatctcttccagctttttctcttttgttcccttgttttgatgatgggttatttcgggggaagctctggctggccttgctttgtgtcatcttagggataacaaagaggatgaaagagatcaggaaaaccgagaaggcagaacagaaccagcagaaactgtgcttgaggaatgaaaatcacctacacggctccttgtcatatgagactgtggcccagcctcctgcaaagccatttaagagtaacccagtgaagctggtgagactgcctgccgcgtccgtgggcccagtgactaactcggtggcttatcatctgggcccagctcctcccctggcatcctgatttcacttggaggggcccccgttgtccttcataaacatgtttatttcattttatttttatgttttgagacagagttttactgttgcccaggctggagtgcagtggcgccatctccgctcactgcaacctccacctccaggactcaagtgattctcctgcctcagcctcctgagtggctgggactacaggcgtgcaccaccatgcctggctactttttgtatttttagtagagaccgggttttgccatgttggccaggctggtctcaaactcctgacctcaggtgatccacctgcctcagcctcccaaagtgctgggattacaggtgtgagccattgcgcgtggctgtaaacgtgatattcttgagactttcagtgaaataagaattgccacggacatctgtggtcattgtccacttgccactcacctacccccttttctggcagcaacagccggcatttcacatgtccatcatcggacagcgtaggtgggaccatcagtcatggtgtcctaccctctgtggccaaggagtggacacaggacccagttagggcaagcagaggctccccttggaatcgcaaagtgaagctggatgccacccacagagactaacatggtgaagctgctgtagcccctgctgttgagcccccagcactgcctgagttcttgcactttgtgagtccagtttaatatctgcttttcctcccattcttggagctcccctcacatctccagtggcttgaagttgccagagatgtttctgggcttgtgaccaaatgactccttttctgcttctcactgctgagcagacacatgtgcgctcactttgcctgctgagtcttgggacccggaagagcttttgggagacaatcacggaccagccccctcttgcctgccctgctgtctccctccaagcaggaggtgagaaggtgtccacctgcagccccggccaggcatccctttctgtgcttctgcccaaatctgaaattcccctctccttgggacccacgactggggccagcctgcctggggagggaatcccagctgcagaaagtcgggacagtgtgcgtgtaaacatgttaatagaaagcagctttgagggcagactagttcagcttcagttacaaactctttccaaatgcgtttaacatgagccactggctgtgcgcagcatatgtcaagctttcatccaatggtggcattttgtccctgcggggtttttttttcctgagcagtttggggcaggggtggggacagggagagagaaaagtaaaaagagagcagtttggtttcttcaggctggagtacaaggcagaggtaatgggatgtattgaagaaggtaggagggaaagttactttagctacagctatttgtccagctgtgctgattaagaaacttggagaaaagcatctttggaatcatgtccttcccatcttatatacagcctttgcagatttcctgctgttctgagagagatctgaactccttaccaggaccttgagggccccacctgattgggcacccctcactctctctgcccctcctccccttcccctcctcccctcctttctccacccccacctgctctgctcagacaccccttccttggttgcttcccacaggccagggctgtcccctggggccttggctgttcccctcccaggagcgcccctctccagctcctcatgcagccaaccttcctgtccttcaggcctctgattaaattctgccttagacatctctccccaccccgctgtgtgaggtagcgccccatgccccagtcccctcaactccactgcctcactttggggacacatcaccccagggacaactgcattccactcttggtttttccctcctcgtctatttatcacaatttagagtcgcctcactcatttgtcaaatgaagttcatctctgcagctggactgcggggttgggggcacatccggctgtcggtcctcaggtaggaggtgcttggcaaccttgttcagagtaggacgttcacagctgtctgccccggaggaagcaagggcacccgccacatggatggaattgaggggaaggcacccggggctcctgcatcgagcttccctcctatattcaatgaggaaatgaccctgcagaaggctggctgcagatgcccctgcctcccggctttgcctgcttggagtttgatggacacgtggtcctgtcagggctacagcaggtctatggtctttggtaacggaaagcgctggtgaaacagtgagctttcccgtgggtgcttttccctgacgccaacaaccaggtaaatatttggaaacggccttgttgaggcttgtgaggtggttttcctccctcccctgtaggcctgcgccacccccccaaccccacggccacctttgggccagatggcacccacagacctgtttgaagtggccacagagggagccctctgggcgctggggccgctgtgtttgcagagggtcctcttactgctgagctggctggtgcagtgagaaggaaggccgacacccctgatcctcatcaagttcagacgggggtcactgcgggtgaggggcctggggccttttacatgtcccgggagctgctgagcaggccactcttctccaggccaccagaacttggccctgcgcatggtgaatcttccctgagtcagctgagtgagggggttcaggcagccccccgggacatggcagtggcggggagtggactggggtggtgcttgccatgactcacgccggttctcctcaggcaaccggatggtcagatgcgctgactcagtggcctgagctcgtccaaaagcgaatcagagaacacagggcctgggctcacccgctgccctcttctggagtcatctgtcactcatcctcatgaaggaagcgcctgggagcctggaatgcacatcgcactgccccagctcccctcttgtttctgtgtttttccattttggattctttcccccaacgccttctgtactgggcattttgtggtctcttctttttctccgagaactctgagggctaccattgcatttgctaatgatgccacagacggtgttgacgttatgaggcttctattactgtattgatttttaccatttttagggggacgggaatcaatatttcatgagggaatgtgaagccagacagtgaagtagaagctggcttttattttgtgccaggctttgtccagaggcgggtggggacgtggctcctaagctcttgattgcagctccttctggcttgggaaacgtttcagttccccaaactctcagaactggatcccctgtgtgttctctggcccggattcaagaacttagttgattgtcaaggaaattctttggctatatttttctcttaatatggtaatgccttttttcactctggcactctcttttcagggaattggattaagactattatttatgggtctgacaaagcagttcccaagttgttgggactggatttgtttaggaatgtctcctgtcctcttcattgaggggggaatacaaattgcttccatttgacagtttatcaagtgtgtgacagagtatcagagtccagggttggccaactacagccagtagtccaaagctggccctctgttgttgtaaataaagttttattgggacatggtcatgctcacttatttaggtagagtgtatggctgcattcagtctacaccagcagagttaaatagttgtgatgaagaccacgtggcccgtgaagccaaaaatatttgcttcctggccctttacaggaaaaaaattcccagccccagtggcaggcaattaacaccttgtcctcgaggagctgaaagtggctggaggcaggaatgcttataagaaccaagcgaggtgaagcactaggtggccgcggcgagcaggaagagaagctgattttgtttgccctttcgtttgccagagattgtgggttctttttttttttttttttttttttttttttttgcagagatgaagctttgatcttgtcacaatagcagagggaggccttatttttgtctatttctctgtgacattggtagaaaggactttgtcagaattccaagctatttggcaattatccaattttgagatcctaatggatctttcgaggtctagtttgttcattcttttagtgattccttgttaattccctgattttataaatgtgtgttgaacatctgtcttggccaaatacttcttaggtgctgaggatgcagcaatagtgggcaaagccatggggcttaagatctagtgtgggaaatgggtgatgtaaagtaaatatggcgataagtacagtgcacgaagcaaacaagtgaaggggtagaaggtatcaggctgcaaagacagcagatagtgtaggcagggaatcttatctgagggggtgacatctaagctgagatggaaaggacagtgagagccagccaaggaaacaagttgggtgacaagagttgcaggtggagttgcttaatttcccacttctgctcagcctgcagatcctggatcttggactaattgcaaactgtcatttcctcgtgagtttattagaaccctccagaacaagtttctggttagctagtttctctgtgtgttgtctcatttcttgttggttctggttctttggggttcctactcatactctggaaagctccagtgtcttaagtagtcagtctcccaagagtctgaaagcacaaagattcacaatgatacgatcacctctcaatcatagcagcattgatgcagttccgtagctggtttcctaaagccatccagatctctttctgtggcaagagagaaataagaccttctggtgaattgaggactaattatcctaataaacatgcgaattaacagttcctttggttaaacaaagcaccagaatctgataatgggaacatgtgactcatggtatttccttctttgctttatctaccaggcagctcacagaaaccactggccttccctgtgttcccattttatgtcataaatatatatttaattaacttattataaaaggccctttgttcattgaccatatcaaattattcttatatagaagaggttatacatgttttaaacattttaaaataaatctgaaaagaatgctacatcctgggcaacttccctgcatttggggctcaaagaagctctatgtggttatgggtaatgaggagccagagtgccttcagggcagttcagcagatgctgaaaggctgctgtgtgctgttcgctgggcccaccaaatagagtaggactgagcccctgtccaccatgacagccgggagatacaagctgttccctttgcctccctgagccctgagctttatagcctatagacagctgaaaagcaggctgcatccgttacccagtcagttacccagacccaaatgccaggccttggctaaccccagttattacctaattttaatatcccaatggatgttttaagacctggctggttcattctttcatttatttacttattcattgattttgtaaatatttctggagcatctgccatggccacatgctgttgtagcagcatcagccactctgaagttggtggatgaaaggggatgcatcaaaggcgctgatgtatggaggagacgcaagttagacttgaccaagacaatattattcctcctctggatgccccgaatatatacagtcattagctgtcgggcccccatgtggcactgttgacattttgtggtttaaacactgaagagtaagggaatattggaaatggcaaacatctgatatagtgtaaaggagactaaatattttgatggtgttcataaacaccgaggaggaaagtcttttcatttttttcatttgtgtgctctctctttctctgtttttgcacactgtcctctgttctccttctccttctctttttccttttttctcccttcatctccccatttatctgatctctcccacctgaaccccttctaccctgctgccctcctgtccattctaccttctctactcccctccctagacagtagtaatcacatgtcagttggagaaacatgatggcaacttggtcacaccgttcttctcagtctgtatatgtcggtgatctcagtgcccatctggcagatccttcctgccctggctcttctgctcactgcgaccacccttgactttgtgatcactgataaccttcaccttctctaatctaaatcccaagcttctcactcttggtccaccacctcccagccttgtccgttctgaaccctgaacggaagctgaatggaaccctgaacggaagggttctgaagctgttcagaaccctgaatggaagctgaaatatcaatgggccattgcttttcacagtcctctgtgaaagattactggccaagccagcatctggagaattcctggtccaccacctccctgtctggagaagctggaacagccagctgcatgagcatgtgacccgtgtactcacaggccctgtgccctgagctcgctgttttaattttatctttgaatttgtatttttgtgaataaagccctatgagctaatggagcatgctcagggaacttggggctttagctcaggctggattcctcctgctgcctccccagtccctggtcccctgagaactccagccccatctgaccttcccttccctgtctctatgcaggggtcattgctaccctctatccctggaaaggatgtaggcacagggcagttctaggttccagcttgggcaccgcttaacatcttggtggtgcagggatcaggctgatgataccgtggttgttctgtgggctactgggcagggtcaagccactcccaccctgatccaggtacctaatgcacccgacacagaagcggcagtgtccttggggtcatccattatccatgtgttggaggagtgggaccctagggaagatgcttggctcgacttccccacccctagccagggcacaatcagaggtccaggggctggtgggcacaatgccaagtcgtgaggcctccagtgtctgcgctcactgtcccataaataaccacagtaataactagcaaatcaaaaacattgtgataggtcgagagagacagcatgtggaagaaaggaaaaagctttctattttagtacctttaacagtgctttctgtatgctttatgaacaaggagcctgcatttttattttgcactgggctctgctaattttgtagctggtcctgccccctagtagctcaagtcagcaaatctttggttcatctgagtccacagtccgctgacccgccctttttcacagttcctcccctgcccatgtgctcacttccctccttacccagcttggcgcactccctcaagcaagtctttggatgctgacatcccccgtaaacaacccttctgcggcctggtttgattttccttaggagacatgcaagttctatagcactgtttcttgctgggtatggaggatgtgctattttgtccattgcatattttttaaagaaaatgaaaggttagcataactgtttccagaaggcacattgaatcactcagttgagtcccagccagttgctgcaatgttagcctttgaagcaaacttgaaccaacacaggaccagcctagaagtcccagcctccagaaatgatgcagtggattctgcagattcagcaacaacaatatttttgtaactcaagagcacttagtaattttcaaaggagagaaagaagtaattgacttggcttattaggttgaaaaagagttgccaactttttctttggttttgatgttattggtttttttttatttttcttttctccaagcttcagggaatgagattgaatgagcactcaagtgctactaggcagaaccctgaatggaaggaagctgaaataccgatgggtcattgcttttcacagtcctctatgaaagattactggccaagccagcatctggagaattctaggaacgccccctcctcttgcagcagtataagtttgcggggatcatctgaccccattggggagttgtatgaaaaaggggatttattggggaccctgttgcctgtttggatcttacttacatttaactattgtctgctaatggattttttggaaagcaaccaggttttccgtaaagaatagctaattgtcagagctgagatgaccattggagatcactgggctcaactccctaattttagaggtgctaaaaccgcaatccagagaagctaatcaagtggttcaaggttgtagactgagttcatataggaccaagacccagcccagatgtcctactgtctgggacagtgttctctcagcatacgtggagcctgagggggtaatgtgtgtgcgtgtgtgtgcatgtatgcatatacacataggtgttttgcctaagttttcacttctgccccaccttggttgatcttggagaatgagcctgaggcgcgctgtcaacctgggggcctcattcagcacaggcccaacttttctgccctgggggagttccagcagttatggttcatctgtggttcagttatggaactcacaccacacatagtgcccccaaaaccgaggctgcgtgcacagacctcccctcccttcccgtggtgggcccctgcttgggttcttcctaaacttcccctttgccctgctctgtgttataccctctctggtcccctgtccctgtggagtgatccggggcacaagggcagctgtttccccgctgacctctgtgtgccctgagcatctgggaggtggggagcaggctggtgagaagaacacctggagtggaggttggggtcagggagggtcccagtcccggtaccacccccacctgctgtgggacctgcagtcccctcatcagcagaacggctatgaagccatcctgcccatccacagggtggtgggtcgtgaaggctgcatacctggcagagcgggagaagctctgggaagatgccggacacgcgccgtgggagtgatttccctgccttgcccagattctgctcccatcacctgaacctgcctgtcaccaccatggaactgctgtgaccattgctttccttttaagcagattagcagacatctcctgctccaccctgccaaacaaacaaacaaacaagcaaacaaacaaacaaaaatgtgcatgagggagtatggacttgtagagtcttttctaaacattgttaggtgcttgtattgggatcctctcttaaaatgaaccatattccccaggctttggatgacactcatggttgcccaccctccaacttccttccctgctggcagagccctgggtttgttttagttccaaccctgaccccaccgcattcctgactcaggcaaattcgcagggtccaatgcagtcaggggagccacgttccctcctccaacgagtgctgaggtcgctgcttgattggatactgccgatgacctacgaggaggagggtgccagggcgcttttgggactttgcttttctggagagatgcttccacagcatggtcatggacacagtcacgtcttgatgtgatgtctggaatggtggtggccgtcttgtggctgtgagaacaggctgaggttgattggatggagggaaggaaggagccttgttcttgatgctgtctgtgagcctttgagttatcagcctggtaccacccagcccttggacagatatctactctacatactccatttggagtttttttttttttttttttttttttttttttttgtcacttgcagttgaaaacaccctaattgatacacacaaactatttttagtgctggtctgtgtttggcccttatggaagactctgggctgagctgcccatggtgagggaggtggactttgtgttttcttactgctctgtgtcctggtggcttgtttgtgtctctgcccatgagacaaaagccgagagggcaagggcagattttcttaatcatatgttccctgcaccaagctcataggagacactcactgaatggttgttgagagagttctctttcacggaggcaatgttttgtgaaacgatgctgcttgttgttgtctgttggttgtaatatgcatgaacactaagagccatctttaatcatgctgtgggccgcctcttccaaggtgttagcattactcccactacctggtcagcatcctgcctatggctaggactttgcaatttacatagatatggtggggagacctggagcccatggccaggactctgacaccctcactggatctgtttctacatctacctggatggccgtctaggacattagaggatttgtgtcttcctaaagtccctctgttgagagacttctggctctgttaagaggacactatttagcattgtgagtccctgcaggctgggggccagtgggcgtttttcttctagatgccccctctcttcttctggcctcccaggcttcctgctcctgagattgtgagaactggcctgtgctgggctcactgcagaaagactgtcgtccccaaaggttttgcaccaaacttgagctacaagatcttttagggggacctgagatctccgcctgggctctatgagagcaggcatgggttgtttttgccccgtcactgcagtcatgcccacacttgcattttcttttccccccagcagtgtgaggatctggcatgaggagtgggactcgcgtgccctctttcttctcctcttccctctggccttttcatccgtcagtgggggacagatgtttgccctgtttacttctaggcttactgtggggctccagggagatggtgaagtggccaaggagaggagctgccaccttcaagacggcctgtggccggtgccgctttaaagggagactcagaggtgctttgctgtgggtggcgcgggaaccagcctggggacagcagtgcagaggccttggactcagagtgcgtgggccccgcggggcttcacggcgcctgtggctgtgcacttccagccatatctgtgctgcatctcttccacattcccccatggagctgatgtctagacagctatggaattaaatgctcaattaccgagtaggaatttggccagcagaggtatagctgctgagtagacagactcgaggtgaggctcacggctgagaacaggccccatctggctttggaatgagctgaggtgcccgatgctcctgcagccagtggctcctgtggggagctggggccgtgacccccaaaaggcagcttgacctcatggaccaccataaatctggcctggtcaacatctctgccagacatcattcccttgcaaagatttctgcctgtgattggaattctggatgaacatgtactgggcgtgtgggtctgacagctgggaagcttgttctcttgtttagccaggctgcccatcatctgtaagcctcagtatccacatctttaaaatggggggaaaatatagctcaactcctaatggtgccatgagaatactttgtcacctgccaggcaaaagcttattcctttcacagaaatccagggtttacaatgtgagacccctccccactccgccgcatgtgtctgcttgcttttttctgtcttagggttgcccttcatgagctaggaaatgtctgagtggatgaaaacctaaacgagatgatcactggtggtgcccattggtgcagcctttgcctaaatggctacttacgtagccacatttcctcgtctgtgttcaggtgaggactggttcctgggcagactgcctgggtttgcatcacgggtgtccatcttgtcgaagcccatgtggtcacccaagtgtgactgagccaggcttgcccacggggtgctctgggccccattttcggcagcaggcagcgtcccctggaggcctggccctccccgggagcatggggagtagcgcctatgggcaagcagcctgcagcctccatccctgcctgggggctcccccgccccagcctcacagcttctccaaaagtgtttgtctccttgccgcatcctctaggcctgagctcagacggtggaaaagaagagctggaaggagagttgcctttcagtctctctgccttctgaggtctcctgagacatagagcctgggcctgcctccctttctaggaggcgccaaggggtggtaagaataggggatgagtgagatgtgaattaggatccccacagcaagccctgcctcgtaactttctgatgggttttcaatgtgtggtgaagcagacgcctgctgggcccccttcctgagttgagtttgacctcctgcctcctgtctatctccttgggcagccaggccaccccgctccattaacctgtgccaccccatccctttacctgtcgcaagcccagccctgaaggcctcaaaggcctggtcttccagccagtccagggcctgaagggatggcagtgtccctggtggacctcccctggtgtggcctagtgcacatcccagccctgcctcctgccccgcctgcacgccatgagtgctgaagtcatgcctggcaggggctgctggcccaggcccagagtaaacacactgcgctgagctcgctggtgtgctgctggatgctgatgagcttgaggagtgtgggaagtgagcatggggctgagtagagatgcggcaggcctgcacctccccgcagctgccctgcatgctccagcctcaggcagccacacagggaaagggtcacccactgtcagggcagacctttaccatggctgggtgacacgggctggctgtggaaaggtgtttggtggttcccgctgttggatttgcacaggcccagatgctcacagcaaaaccaacacctagatggtgcttacaggagccagcgggtattcaaagagctgttcagatcttaagttgcttcattctcacagtggaccattgaggtagctgtacgttagtcccattttccagatgagaaaactgaggacctgagtggtcataagctcaggccctcatctaaatcacgcagcctggccccaggtgtgtgctcttgaccatggacagtgctctcctggtcctcttggtatctgtgatctgagggaccttcctcctcctcagtctcgtatagtcagttttaggtcttggactctgtcttcatatccctttctcccttcgtgagctttctcacccagcaccttccttatttggtgtgtgttgggggatatttgtggtgtggcgtggcactgtgtagtggatgagagagtctgtttttccgatcccagtcccaggtttcaaaccctgctctgtctcgagtcacccagaatcttggaccctcagtttcctcatctgttaaatgggcatggtggtcaccccacctcatcagctagtgtctgctccatccctggtggaggagatgactcaagtaacccctgggttccacctgccccaccccactggtcccctggctctttctttgttgagatagacgaatgtgaggctctggagttgcagttcccacgagggctggggtggctgtctgatttctgggcctggtccatgttgttcagggcagctgctcgttctaagtgaataaaggctgaaggaactcgggaggtctgctcggctccgaggaaggcagagagggaaagggccccgatgccttccctgatagagctagggaggcccttctgtggttccccccagctccttggcctgggtgaccctggagctggcttctgttccattttgttgtgcagagttgtttgagactcctggctttgcctggcctttgtgggacgctggagatcagggcttctggagttggccaattagcctgcccagaccaggaagcacaggtggctgacagagggccgtttcaggagaggagagacagcctacctattcggtcttgctgtccccatgctccatccctgcccctgaccagtgtggccctgtactcagcataggcgtgcacctgagtcagtacagttccctgcccgcagagcaccccaaatattccaggcctcaggacggatgtgcacatgatgagtcggggcaggtttcactgcctgtagcttgggatccttccctggggcttggttctctagggccatccccagcagtctcaccccaaaccctaaattcatgttgtcttcctctgtctcttggcctcaaggtttcagagtgagtctgtgctgatagcttcaagatgtgatgagaccccgacttggcctccagttacctccccacggtttccttggtgtgtgtgtggcttcagtgttcactggctcccgcacggcttgcaatgtgtggattacgggtgggagggaaatccagtcctgcccgcagcaaagggatgttagttgtgagctcagttccccaccgggcctggtgtttccaaatagcccgtcactgtccctgcttggttttccatgatatctgtgcctttacctatttggttaaattaaaccaactcagcaacgccagccattgtggtttcagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaacgtgagcctctgtccctctgcgggtggggattggggcagagttttgccagggggagaggagtcagcataggtcttagcccctgactttgttgtagtctgcgtgaagggatggaactagaccaagccatgtggattctagtgccagcagcatggcaggggtcacatggcggggacggtgacaccggagcaggtggacagccagcctcctcccaggaggaagaagttgtattgggtgctttagggtgattgcagttggcttctgggcttcagagagaaaatctccctgtttacggcacctctaaaactttctgaaaattgttaaggtcatttttttccggcaaaatattaggttaatgggaatgaatctcagagaagaatcgtgccccccactctaggcaccgtgctcaggaaacgaccaggcagggacatagattgaaccatgttatgacacgatttgtaaccttttcatttctgtttaattgcagtatggaaactcgggaggcccgttagtaaacctggtaaggtcttttaaacctatgttaggtcatttgtttttatctatgtatacgctgttttttgtttgtttgtttgttgtttgtttgtttttgaggcagggggtcttttcaaacataaggttgccaaagtgtattataaattcctttaaaatggctctgtaaatgtactgcgtgcttgcaaatgaccctacggatcttttctggaaagagtaaggcaggccggaggtgagggttggaaatgttatgccagagaacacacttgtgtctcagagttacaggtaaacaccgtgaaattcagggccaatgcaggagtaaggtgaaggtcactaaaaatgctggccagtcaccgaaagcacctcctccaaattaaatctcctgggctgctgaaggagctggctgggctcatacacattttctcttggccaggaatcctcccttaaggcctggctggaatgaggaggagttacccacccacaaagatatcacttaagtcttcccttaaatacttgagcagaaaaagtgaagccttagaacacagaccagcagagctagagggcagctctggggccatttatagagggcagctctggggccatttatagagggcagctctggggccatttataggggctgtctttagcaaggcccagtgtgatggcacctcctagatggtgccttggcatcaggtactgacatctcagcactcctgggaagtgtgcacttggcagctttctcttcccagcagaggggcagctgtgctcccagctctgtcctctgcctccccgcgcagcacttggggatggagtggagatggctttgctggtaatgaagcatgacagccctaagctctagggttgtttccccctgaagtcagcagagtcatcttaagatcattagacatgggagaagcaggaaggtgtgggcagccacctaaaggagtttgagcctttggaaacgtattccttgtgaaacaggagcaaatcatatcgtgcattttgaaactatctgtgcttaccgtgaggtgagcacccagtgccgacctggagtatgtgcgattcttccacagctgcgcgtggctcgcgctgcctgggtgtcctgatgcctctctccctgctgccacggggatcccctccttgcatctccccacttcgatctctgaaatagctcagggacttctttcaggcatattctctctgggtgtgtacctgccggtaaagcttcacgattcagtaagccgtgtccttcttgcttttcaggacggtgaagtgattggaattaacactttgaaagtgacagctggaatctcctttgcaatcccatctgataagattaaaaagttcctcacggagtcccatgaccgacaggccaaaggtaggcaaggcccacacagccctggggactccggagatggggcctgaagctcagctgccctttgggacttggggaagggaaaagcggcagcccctaggactagccaagccgtctctgatccagaagtgaacgggaatgcacattactaaatccctcgcagaaggtcacagacatttcaccatttttgtcctctgatcatggcaatgtcacttgagtcagtctaatatgtaccaggcatgatcctaggtgacttgtgtacattatttcactttctttatgtatgtcacttaattcttttgccctatcagttaggaattactagtcccattttgctgatgagaaaacggttcagggagatcattctgcaaacgtttattgccccatctgctctaagtcaagcagggagcttggcagtggacagctcaactggggcctggggctcaacaggggcctttgccggtgtgacttttatgttctgttgggggatgggaaggctgacagtaaataatcaaacacataagatactattagtgctcccaagaaaacggatcagggtggccgtcaagggagcgactggaggggcagctggtggagatggtgtggccaggaaatgccttccaagctgaggtctgagtgaggaggaaccagcgggcagggatgtggggggaacactccagaaggaaagacagaggactcagcatagttgagtgagcacaaggcccctgaagtggcctgagggccggagcacagtgacagcatggagttccccggggtggaaagaggccaaggccgggcgagcaggctcacagcaggccgtggtgagggacctgggttgcatcctaacgacatttaagaacagggaagtttatgatctgattgatgtcactgaaaggacactctgatggctgcggggagtctgctggaggggttgctggaagttggggaccggttaaggggctctcccagccatctggatgagacatgctggggtctcagacaagggtggtggcagtggaggtgggacagaggggtcacattccagatatatatggggggtagagcaagcttggggaagggccagctgtcaggatgaggccatgaggaattaagggtcatgcccaggtacctgaccattaattgaaacaatgggactttcccaaggtcccccagaggggaggggtccagaccaggatttgagccgcaacctcagtgtacccttctgtggcccttcctgcaacctgggggattgggcccccggcccctggtgtccccagcacccccaccaactgggctgaccttctgctgtccctttgttgtctcaccaggaaaagccatcaccaagaagaagtatattggtatccgaatgatgtcactcacgtccaggtgggtaaacaggatgcgtgtctgtgtcttaaattttaataaacctgaacttcagaaggtgctcacgggcacccctgaaagagaaaccttatgctgccttaagacgtctcagtttctgcttataatgaagtagcatcgggaaagaggacaggtcattagccttggcccctttgtttggttttaacctgtgtttttgcattctgagctggttttcttcactggcagcaggccctccggtgtagaaggttctgccctcctctttgaaggcaggcctgaacagtgtgtgcgtggtggggctgttgattcactctggctcacgtcttccttaccccacattctgttgaaacccacattccaggagggccccaagcccctcccgcagctctaggcactctgctttcgttgctctgcagctcgtgggccgcggctccaggaatgccagggcaggtccagcgcagggaagtgaatgactgatgtgcttgttttccccgagctggtggaattgcggcctgtggttggcaggctcatggcatcctggtgttctaaactggatgaaaaattctggtgtaatctcatgagtcctggtagtagactcacctggcatggctaaaactgtcagaggtaaagtaggtaaagactagaatatagtaacagatagattaatgtgttcattactatgatgaattaatgattcactcactgtgaaagtattaatatattttgatacatgttatgaatggtggtccctttcttagcactccagaagatggagccatttgtcaaggttaaagtgtcccctcagttgtttgcctttggaactacgaggtgtagggaaagatggtaagcccttggtgcccagcttcctgggttcctgtccctgctctgatatgtcctgccttgtgaccttgggaacgatatgacccctgagtgcctcagtttcctcctcttcaggatagggatgacagcgcaggtgcttctgatgtgtggccaggctcagatcagggagtggtggcaggggtcaccagccacagtgatgccagccactatgtatcacacgtactgggccaggtgccttactgggatgatctcatctgatcctcacaactcatgttgtagggtactgttattatccccattttgcaggtgaggaaatgaaggcacagagaagttaagcaactgtccgaggtcacacagctagcaaatggccgagctagggctgcaaaccaggccaaccactgtactttactgactccttagtaatagctactattaattaagaaataataacaatgatgatggctgggtgcggtggctcacatctgtaatcccagcactttgggaggccaaggcgggcagatcacttgaggccaggagttcgagaccagcctggccaatttgtgaaaccctgtttctactaaaaatataaaaaattagccgggcttggtggcaggcacctgtaatcccagctactcgggtggctgaggcaggagaattgcttgaacccgggatatgtaggttgcagtgaactgagatcgtaccactgcactccagcctgggcgacagagcaagactctgtctcaaaaaaaaaaaaataaataaaaaaaataaataaataataaagcactttccttgctgttaccaagtaaatctttgactctggtagacaggcaattttaattttaaaataggatcagaattcctggaggaattttaccttagacctaaggagaagacgggaactggtgagagctgagttttgcgtgaggaaggcctggtgtttcttcacactaacacgggtgctttttctctggagcagcaaagccaaagagctgaaggaccggcaccgggacttcccagacgtgatctcaggagcgtatataattgaagtaattcctgataccccagcagaagcgtgagttggagtcgttttctcttttcccaatattcttgttgttcctgtgggggtagcaggaagagggagcgctgttccttttctactggctcagatgattatgttgatccttgacagacgtggtcggacgttgcttgtcattcctgctggccaggccttccgacctggctcggctcgggactcatccataggagggtgccttctgtcttcaaaagtccttgctccacgaggaccctccagatggacagagcaatagcagactcgtaatgagtctctgagatggcccggctggccagagagagggtttcaggaacagtgtccccaagccctcacttggtggtccttttctaggcttcaggacccttctcttcctggagtcttccagaatgtctctgacaattaggcccatacctgtcaacacctccagaaaaataacccaagtgatatcaaagtaacatgacaagaagtagctcaaccatccatcagggtttgttacctgtattggcggaatatccagagaaaagtgcgagaccagggaccagcaaatgtgccttgggggctggatctggcccactgcctgcttttatatggagctgtgggctaagaatagtttttgcattttatttttatttttacttattttttattttcataggtttttgggggaacaggtggtatttggttacatgagtaagttctttggtggtgatttgtgaggttttggtgcacccatcacccaagcagtgtacactgaacccaatttgtagtcttttatccctcatccctgtcccagcctttccccttgagtccccagagtccattgtatcattcttatgcctttgtgtcctcgtagcttagttcccacttatgagaacatttaaatggttgaaaaaatcctgaaataagaatagtattttgtgacatgttaaatttgtatgaaattcaaatttcagtgtccactgtaatttggtttatgacatctatggtggcttttgtgctggaacagcagagttgagtagcttcaacagagaccatatgtactgcaaagcctaaaatatttcctatggagccctttacagaaaaagtttgcagacccttgtgctagcccatgaaggaccatgacagcgttttgacgctgagctatataagagctacagttatagtggcaaccacacaaaggaagtgcctcttaacagaaacattccgcccacccctataggaactgcattctgagttgcaatacccattataagcaagttggccagatagtggccaactatctggcagatatctggccaactacgtggcagatagtacctggtacatccttccccactttggggtcaatcttgacctttgatctccttggggtcataaagccacacaagtgttagtaggcatttctacagtggacacaatggatgatttagcctaaaaatctcaaaaggagcccagcatcctggcacatgcatgtaatcccagctactcaggaggctgaagcagaaggatcccttgagcccaggagttcgagactagcttgggcaacaattgagaccccatctcaaaaaaaaaaaaaaaaaaaaaaaaagagtggggaaaaaagaacattattaaaaaaaaaaaccttaaaaagtaatccaatctaccgatggtttattttttattttattttattttttttgagatggaatcccactctgtcacccaggctggagtgcagtggcacaatcttggctcactgcaacctccacctcctgggttcaagtgaatctcttgcctcagcctctgagtagctgggattacaggtgcccaccaccaaacctggctctttttttttttttttttgtaattttagtagagacggggcttcaccatgttggccaggctggtcttgaactcctgacctcaggtgatccacctgcctcagcctcccaaagtgctgggattacaggcatgagccaccgtgcctgacccactgatggtttgaattattctaagttcgccaccgtccaatcctgtttgctctgggcttttaggttctaagctgtgcctctgtccatgtaaagtcagaccaggaggaatggaaacacgaaacattgccattgtgtttccctttgtgttgcagtggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgc

SEQ ID NO: 3 - DNA, 2123 bp, Macaca fascicularis
atgggctgggccgcgcggccgcgcgcactcgcacccgctgcccccgaggccctcccgcactttccccggcgccgctctccggccctcgccctgtcagccgccacggccgccgccgccgccagagtcgccatgcagatcccgcgcgccgcgctgctcccactgctgctactgctgctgctggcggcgcccgcctcggcgcagctgtcccgggccggccgctcggcgcctttggccaccgggtgccccgagcgctgcgagccggcgcgctgcccgccgcagccggagcactgcgagggcggccgggcccgggacgcgtgcggctgctgcgaggtgtgcggcgcgccggagggcgccgcgtgcggcctgcaggagggcccgtgcggcgaggggctgcagtgcgtggtgcccttcggggtgccagcctcggccacggtgcggcgacgcgcgcaggctggcctctgtgtgtgcgccagcaacgaaccggtgtgcggcagcgacgccaacacctacgccaacctgtgccagctgcgcgccgccagccgccgctccgagaggctgcaccggccgccggtcatcgtcttgcagcgcggcgcctgtggccaagggcaggaagatcccaatagtttgcgccataaatataactttattgcggacgtggtggagaagatcgcccctgccgtggttcatattgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaatggtgccacctatgaagccaaaatcaaggatgtggatgagaaagcagacattgcactgatcaaaattgaccaccagggtaagttgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccggaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagtttctcaccgagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatatcattgaagtaattcctgataccccagcagaagctggtggtctcaaggaaaacgacgtcataatcagtatcaatggacagtcggtggtctccgccaatgacgtcagcgatgtcattaaaagggaaagcaccctgaacatggtggtccgtaggggtaacgaagacatcatgatcacagtgattcccgaagaaattgacccataggcagaggcatgagctggacttcatgtttccctcaaagactctcccgtggatgacggatgaggactctgggctgctggaataggacactcaagacttttgaccgccattttgtttgttcagtggagactccctggccaacagaatccttcttgatagtttgcaggcaaaacaaatgtaatgctgcagatccgcaggcagaagctctgcccttctgtatcctatgtatgcagtgtgctttttcttgccagcttggtccattcttgcttagacagccagcatttgtctcctcctttaactgagtcatcatcttagaccaactaatgcagtcgatacaatgcgtagatagaagaagccccacgggagccgggatgggacggggcgcgtttgtgcttttctccaagtcagcacccaaaggtcaatgcacagagaccccgggtgggtgaacactggcttctgaaatggccagagttgactcttttaggaatctctttggaactgggagcacgatgactctgagtttgagctattaaagtacttcttacacattg

SEQ ID NO: 4 - DNA, 52575 bp, Macaca fascicularis
atgggctgggccgcgcggccgcgcgcactcgcacccgctgcccccgaggccctcccgcactttccccggcgccgctctccggccctcgccctgtcagccgccacggccgccgccgccgccagagtcgccatgcagatcccgcgcgccgcgctgctcccactgctgctactgctgctgctggcggcgcccgcctcggcgcagctgtcccgggccggccgctcggcgcctttggccaccgggtgccccgagcgctgcgagccggcgcgctgcccgccgcagccggagcactgcgagggcggccgggcccgggacgcgtgcggctgctgcgaggtgtgcggcgcgccggagggcgccgcgtgcggcctgcaggagggcccgtgcggcgaggggctgcagtgcgtggtgcccttcggggtgccagcctcggccacggtgcggcgacgcgcgcaggctggcctctgtgtgtgcgccagcaacgaaccggtgtgcggcagcgacgccaacacctacgccaacctgtgccagctgcgcgccgccagccgccgctccgagaggctgcaccggccgccggtcatcgtcttgcagcgcggcgcctgtggccaaggtactctgccgcgctcctgggcagcaccccattctctccatcccagctcggacctgcttctgcgggactggtgggcagaccgaggggcagcgaagcgttgcggggtggccagggcaactctcggggacaggcaggtgggccccggggtggcggctttccgcgggctgcctcggaaacgagcttcgcgcccagcccgggccggttctgcgcccagacgatgccggtgcgccgggcctgcactctggggctcgagacgcctggcgacctgccgcggagcgccctgagggcagccacacagcgcggggagccgaggacaaataagaggagtgggggcataaagggaggagagaagttcaggactaggaactggagccttgcagagcggcttcaggaccacaagaagtcatttctgttgctttttctatttgcttcctccgtcccctttaaaatgcattactttgatcacgggaccgctccgtgaaaactgtatgtaactcttttggaaaggaagagtgtttgccggcccccgccggagtttccccaaaaagtctaccccgggcagggaacggtttggcatcgcactcgtttcggcggcgttgctgcctgtgttgctttcctcgttttgagccagccctacaaaaatgaaagtggctccttttgaataagctgaatcgggctttggatcacgaaatctgcagaggcgtagaagggaccgggttagtaatgaggaaggagcctacccctccctcctgccgcacacaggacctgttcggcaggggagatggtggtgatgggggcaggagtggagtggagcaatgtctaactctctcgcgggaccttccggagagatgcttcccatcttcaggcagaggccatgtggaagaataatatcgagttcagcggcggccagtcccgcggtgtagaaccagccagcggggcttggcagtgcgcttaggcgcagccatgcggctgctgcccgaccccagcgctgcctcctcaactcgggcagtgccaggagaggggcataggagagcacagtgcagagggactggtctagattttactttataggaatatggttcagtatgaccaactaggacttggcatagtttggcttacatggaccggaaggtgccagagccgaattgggtgaaattcgagattgtgtatttcactaacgcaggagcacagccctcgggaaactcagcctagttaggcagtagagagttgtcccggagacaagtgatcccgcagactagagaatgggcatgatgatagcacacgcctattgagcactcagtctgtgtgccgggtgtgttacctctgtgacctcatttggtctcacgaggagggagtttctcctctctctctctctctttttcttcttaagagacagggtctccctctgtcgcccaggctggagtatagtggtgtgatcatggctcactgcagcctcccacccctggactcaatgattctcctgcttcagcctcccaagtggctggggctacaggcggatgccaccacacccagcttctcattcctgttttacagatagcggaacttaggttgaaaaacttgcccaaggtcactcagctggagtttaaacccagatagcctcattcagaggagtcaggccagcacttaactccaagggtgtgggagaggggtcaggtgctgtaaatttccgggtgggttggacgtgcatccccctcagagccgggaacagcatacacaaagcctaagacttgtttggaggtgaatagatcagtgtggctgggggatgtttggggagggcagcaggagtgagccaggctgctggcccagagtcccagggctgaagaggctggctgtgccccgggccctgtgtgcagatgttcttgaactggggcaactcaaagcctagtgtagtgtagggctgacctagcagtggtgtgcggaatgcatccagggtggagagtttagactactgcaataatctgggtgtgaggcaacaacattgaaaaagcatgtttttgtccaaaacaagccagctgttactggtctcgctgtttgtggtctcattgcacggggtcctgagttgctggcaccatgcgagtcgcctaatttattgctagtgaggcaagttgcttaataagctttggagttggctgagtccctgtgtggaggaaaacaggtcccccattggccatcaggctcacggcgggccccggtgtaccagtgaggggacagccacagagggataagcatggtggctttgaaaggagggagagacagagtgggtacaatgctgtttttatccctccctccttcttttgcaaatatttgttgagctccgtagggtgtctgacaccgtttgcatgtttgtctggcacaccagaggcacttggtacgagtggattagtgaatgaataaatgaatgaatgaagacaaatgggaggtgctttcgatacacagccattctgtttttccttagtggaaggcactgctttgctgcgccccctctctggatctcactctccacccttgactttccggaggtgtttccgaggacaggcgcctgggagccagcagacttcattcagtccaagccaggctccaggactcagcagctggtgcctacgggcaggtcacttgacgtcactgttaaatgaggtgaattggctgcctgctctggctcgaagattggcgggagagctactttagctgcaatggacatgagccttttcatggggtgccacttgactagaggcctgaagttggagcaaggcacacacagatctgaagacagagctctcgaggcaggagcgggtgctgtgatttcaaatattacaaggaggctttgtctggggcagagcatgcgaggggatgagaggtagaaatgtcatcagatcaggggtctccaggcaggtgaccagtactttgggtcatggtagatctttggatagaggaacgtgtcaccattcaaaggaaggtactttcatttgtaagctgtttaatgaatagacctcagagaacatctctgctcaccgctctggaaatgaaggcaaatcatctatttcagaagtcaatgcactggcagggtttggatggcaaagtatacaattcaactagagaacaaagatctgtcatctccagctctgctggtcagatgattacaaaaaagaaagggattgaaatactaataggatacaaataatgagggctaacatatatgttgtgcttattctgtgctgggtgcatactaactcatttgatcctcctgacagtcctgtgagtgagtgctgtagtcttccctgggttacagctgggcaactaagtcacagagcagtaccttgctcaggactgctggtcccacacaactggatccagagtctcgttcataaccagcatgccgtgccgttgacagagcaacagagattataaaccacccccagctaagccccagctaatagctgaaatcaacagagctccagatggctgtggccttgagatgaaacaggacagatcacagccctcactcagcaggctcaggttgacagggttgcctccagttgccatcagtgcagccctcactaaagaaaagcaaaaagaaccgagggactgtaggaaagctgtttccacgccagagatccagacagcaaactgctcttgaagagagaaagcccttccggattcccccatgtcccaaaagaccagccacgattccagacctctgctaaaacacggacaagaagccaggatcaaaacctgaaacagacttcccaaacagcagaaccctcatccatttctcctcctagtacatcctccaggaaaggccacccgactcctgacaggagcccagacaagcttggaggtctgcaagctgcaggggtgcccagaaactccgcctctggtggtttttagtattgcctgctcctggtctcaccccagagcctctgaaggcagaggctgtacgtacatacctggtgaagaaccaagggcttagacggttgctttacttcttggaggcctggatggtttgtaaaatttatttatttattaattttttttttttgaaacagagtcttgctctgtcgcccaggctggagtgcagtggcgcgatctcggctcactgcaagctctgagacctcgcgagttcacgccattctcctgcctcagcctcccaagtagctgggactgcaggcacccgccaccatgcccggctaatttttttttgtattttttagtagatacgaggtttcaccgtgttagccaggatggtcttgatctcctgacctcgtgatccgcctgccttggcctcccaaagtgctgggattacaggtgtgagccactgcgccggcccaaaatgtactttatttaggtgattctttcatgggagcctcaaacaagcaatcattgttagctgagtgctgaccctgtgctgagctctggggagacagggttgaataaaacaaagtcactgcccacagggaacttacattcaatacattcagtgcaatcactgcttccccaggttgcatttttccattgttagagtgggcggtttgctagagagtcatttccactgttggcaattcaaatacaccttttgtcacttaaaaaacaggtgtgccgggacctgagcttcatcttagggtaggatgggtggaaacagttgtgagtctccagtttttagtcacccgaaacttggaaacttggaattcttttgagcagtttatgaggctctgcctgctctggtcagctgccttcttttattgctctgttggttttgctaaagagttaaaatattaaggtttcgtgaaattaggacgttaacaagctcaaaaaccaagtgtctgagttacttcattccactgagagagctgtaaatgggttgcattggaacttaaaataactgcattgagtaagcgatggtggcgggcaccatgagctaactgtggtcagaagcctgacagcctctgctttggggctggattctccgtttggagctgtgtgatcctggacgagtttcatgccttggatttagaaatcagactttccatgagcttatatttcaagtgaataaatagctctggtcaggcttagtttgaagaagaagtgagcttggcagtgggtgagggttcctcggaaggccagctggggtggaggggctgaggacaagcggctctggcccttcccgggttgttacctgatcaggtaacggctccctcgacctcttgcagcctcggcagtaaggggattgggccagttgatctctgaggctccttttaactggaatggtctgtgattcttgtaagaaaacaagtctctgaggaggttgtggtcgcctcattcctaatttaaaggttgggaaggcttccttaagagctacttctttttcctaaattattgacggttaaagccaaggctggcatcgaatggatgtgatccatcttgagcctggttgctttgtgtttcagctttgtactggctgctgaaagtccccaggagaccacaggggtgacatgttcatccccaagagatgagcttccaagagcctcatacctcttgctccttccctggagcctccaggcctttgggtagtcggaagtgagatacctttgtgtcatttcatcttttccatctccaccttctctgccattgaaaaaaaaaaaaaaggaaagaaaaatcctattaatagagaaaccgagaagtgtagccattctgaatgtgtttccaaaaggctcctggaagtggcatggaagttggagtgattcagcactacttggtgacgtgtgcctagaaccatagggggacattagccaggacaacacgcctcaggacagaagtaagtggctgtgaagaggcatgtccgtcactgctggaaaggcgcagagttcagcttttggagtcaatgctgagagttccacttctaaattcattcagagcatttatttaacacctactgtgtgcttcgaagtgtaccaggtacggggactcagaggtaaggactagtggcccctgatctcaaggtactggtggtagatagtgtgatgctcagcttaagggctgggcttctgaagtcggattaccactttctgaatgtgtggcttttcttgagtgacttcatctctaagtctcagtttccccatcagtaagataatagaagtaatagcagatacatacatagctcttagggcattgcagaatggaaggacctccttatatgaaacgcaaagcactgtgcctgatgcattgctagaactcaggcaatattagcatgttgtcattatcattatcatcatcatcatcttcaagacactgacaaaggagtcagctgtatgggaagagtgctgagacgctcttgtctccctggggatgaggtgggtgggtgggttaggaaaccttcacagagaaggagggtgatgtgagacttgtgtctgggagctgactcggaatttgccatctactatgttggaaaaggttctctgggcagaggtatccaaagttgccttgactatcaccctctgaggtcccagttgttgcctatatcatgtgaccagtgtgtggcttctcttgaattaagagctgcatgtctggactgcctgggattttacagatgtcatcttgttaactcttcctggagcttgtgacacccagaagatggcagtttatagaagccctgggaccttcttgaatgatgcttggtttggtttccatgctctgggaattcctcacaaggaaagatttgtcacatcttaaggaaggaaaaaaaggcaaatttgggagtccatggataccctattattttagattccaggacaaattgtcgaataagcacatttcataaaaacaatcctccgcagcatcccgtgacagcagctggtccctcaccacaggataattatgtctccttgtgcacacaaaagtctccgagggcatattgttgtggctggagtttctgataatttccaaattgaacaacctcagtcctaatgagtcagaagcttgtgcaatattttcaaacctcaggaacatctttttcattagttgtgcaataaagatagtaggcctatctctgtgatgagctgttttttttttttctcaaagtttgatgagattcgctgtagaattccttctcacatagtcttgggcaagattttacccgatcttccaacacatgagtcatatcatatcctgtgactaagaagagctgtctctttggtgccagttttgtaagcacagtcaccacttggtggagacggatggacacagttgggattgcccaggcagatgggcagtcttgccaagcagacataggggagggaaggctcaatgttcagcggtcacatctgcttttctgtggcagagtgagctatacaggaatattgtattctccaggacagttagggcagtgggaaatgtcaccaaacagaacagtgacccaaagagctgctgccactgggtgctctgtgggagctgggcactgtgctctttgtgttatgggccttgctttgttcttaacttgtagccacccagagaggtagggcattagccttgcttcctagctgagactacagaagaggctcctagaggttagctgtaatttgtccaaggtcagccagtgcaaggaggcagagccaggatttgagcccatgtctgtctcactcccaaactattcttcagatttctttaagtcaagtgttatttagaaatgttttgtttattcgtcaaatatttggtggatgtttccagctatctttcggttattaatttctagtttaattccattgtgggctgagaacatattttgtatgatttctattctattacatttgttagggggtattttctggtctagaatgtgatctgtcttggtgagtgttccctgtgtgcttgagaagaatgtgtgttctgtcgttgttggatggagtattctataaatgtcacttaggtctagtggattgatagtgccattcaggtcaactgtatccttcctgattttctgcctcctgatctatcagttcctgaaagagaagtgctgacgtctcctgagtctattctgaaacactggattgcggtctccatgatgaaccactagagttagaaaacctgagtcctagccccatttgggcctttgggatgactcccttccacctcagtttcctcaactacaacaggaggacgatgatgcttcccaggagacatcaacaggatactgtgacataagggatatgaaggagctttgtcaactcctaaagtttcaatgctaggaatcctaaagcattgaagtccaatgatataaggaatatgaaggagctttgtcaactcctaaagcttcagtactgggaatcctaaagcactgaagtccaatgatataaggaatatgaaggagctttgtcaatgcctaaaacttcagtgcttcaggagtcctaaagcattgaagctttaagagattaggacctctagttgacaattccagactcttccaggactcctgatagagccaacaccaagaatagtgaagccggaaggatgcaaatagtaatatgtctcctgggtgtcaaagtgtgggtctcctctgggcatgttctcttgtcctactgagacatgatagctcttggccaaagtgactgaacttgaccctctgtttcaggaaggccaaatgcagggttcaccactgtcatgtccaagggcagatgctttggtccagaacatcagcatcccagtcattataccaagcaagctgcaatctctgcctgcaccgtggagagcgcacgctcctcccagggtggcctgcatcctgtatcctgcatcctgtgttcttctcaggccgactttctgtttaatgtttgctggtcaggaaatggcctgagctgaggtttctcagatcccagcctgacctttctccaccagcatttttggctctgaaaaatatagcccagtgtggtttagccccactggatgaaacccaataggaaaagtctgataatagcagaggaggcgtaggaggaagggtgaggatttgagagcatctgggatggaccatgtgtgtggatattgttctgtctgtgggattgtgtgacacttctcatttacagtctgttcccttggaagtcccatcattggccaaacatatagtccttctgtcctctgaaaagtatcattctgctcctacctttgacaaccatctctgaccacatcaactccctgttttcatgcatcttgtggatgaggacaccaccttacctgtaaggacactggtggcttcccaaagccaccaactgacttgtagagaagacagaatcccagagtatgaagcctgagggtgaagggtcctggcaggtcctagagcccaacccttcacttcacaggtggggaaactgagggagccaatgggaacatgactctcacaagccacacagctcatctgtaggggccagtgtggagtctgtttatcttgagacccagggctgagtctttgagccctccccatctcagccacatcctcctgttggagcagttaggtgtttgggagaggccatggtccatactcatggtattcctgtaaagctggagaaacaggccttgctcccttagtctctctaatcaaaatgaggttgcagaaaacccttctccctacttctccctaaaataatttccttgggttagaagatgactaaaaagctattcatctgatgactgatgtctcccttcaagagttataagcacatataaatgcctttgaatggtaattataataattttgctgaagggaaaatatcagtataaatatcatggtggactcactgatgaatgaggactgaaatgctttcatgtcttttcagctgtggttagattttctttgagcagagtatacaagtttttcctctcctagcataaagactttttttttgtatcttttctctctactgtttagacatgacagaaaatgcatttatacatttgatgacatattgtactatctcagttctttaatattataaatgtaatttaattctatgaaaaattaagaaaagaagattcatatttcaccattaccatctctccagaaatactattattattattattattttgagacagagtcttgctctgttgcccaggctggagtcaggggcacgatcttggctcactgaaacctctacctcccaggttcaagcagttctcatgcctcagcctcctcagtagctgggattacaggcccacaccaccacacccagctaccttttatatttttaagtagagacagttttgccatgttggccaggctggtctcgaacacctggcctcaagtgattggcctgcttcggcctcccaaagtatgggaattacaggcatgagctactatgcctggcctaattccatcatttctgtcccaagtgttgccaccatttggttaactgttcccctgtccacatccatttaggccaaggttgcgatgttaaacaatcctgagatggacattttcatgtttatggctatttctgtatctagggtcattctcttaggagaggtactaagaagtacagaaactggaaagaaggatatggaatttttatggttctggtataaattgccaaattattttccagaaaggttgtagccatatttgttgacatcagctctagaatttcaacctcgtaagtcactgaaagaaattatcccaaaagcagtccttcaggaataatggaagaagatggtgccgaacccagccattctgctcactgttagattacttttttggtcttacaggttactttcattctcaggttgattgctcttaacagttgagcaatgtttggggtagaataatgagcacttttccaatttggttctacctggttgagttgtgatcacaggcagtctcacctgggaggggcttgggtggttgtcagcttgtccttccaacactcgcgtctcaggcgagcagcctgggaccagtgaggcgacctgagggctggaggtcacaaactaggaggtaacagagaacccaggtctcaggaagcccagtccagggctcgctgcagtaagcctctcggatgccagctctgtccaggatgcgggaggaggccagactgatttggtctgttttgaaaagtgatgaaaatatttattcaaatgttttgtacacataggcagaagtataacagaagctgcatatacaaaatcattttctagtagtcacattaaaaaagtaaaaagaaacaaagaacattatttttctttttaaaacagctttatcgagagataatttacatactataaaatttaccccaagtgtacaatttgctgttcttatgtattcacaatcatgcacctatcactaccaactccagaacactttcatcaccctaaaaagaaaccccgtatccattagtagccaccacgtacttctcctctgtccagccctaggcaaccaccggttcattttctgtttctatgaactggcttattctggacatttcatataaatggaatcaaacaatacgtaactggcttctgtgtcttagcataatgttttcaaggttgtccacgttgtagcagggatcattatttcattccattttatgattaaaaataggtctttttatggatacagggagaccagacttctattttatctcccctccctgatggggaatcctaatttcagcccggaaggtcactgtgaaagtctaaacgcacaggtgatactgactggttccattggaagaaactgtagcacctgactcaggaagccagcattaaaaccaagaatattctatacggatggggattacgcactgaaaggaaaacatgaggaaatgcacttttcagatttattagatcacagaacttctttggagctggaaaggatttcggaaaccgtctagcctaccccctcgtcttaccactgaggtaactgaggcccaggaaggggaagtggcttgttttgggtccgggaccacttttcatttcttatttgagccaaagcttccttctggtgtctgtctctgtttcacaagttcccgttgcatgggtgctgggtattgcttgaaaggactggcctcttccttgatacaggggctcgttcactgtcacctccctccctcacgtctcttgtgcccctctgcagccgcaggccctcctcctgcaccaggggggcacactcaacccgggtgggcactgcctcctagtctgcggccagaggctgggaggctggggagactgaacagccccggcagctccagacataacaacctatgttgaggagtcggtgcaggaagcgaacccagctgagaaatctgcgaaggtcaggaccggagccagacgcttatcaagaggaaagttaatggtgtttttgtgaactgagcagtcagctgtttccctgaagataataatagacacatcatgttgggcattcaggaggcatctaaaaaaaaattgtgcagtggaattgattggaagcttttccctaatacataaaataggccagaaaagactatcaaatgtaacagcaccgatcaaacccaagcactcaccatagatccaagcaaggactgaaaaacacgaattttttttttttttttttccgccagtgagtctgaaaagtgattttcaatgccaggcgcctttaaacacagacaacataaacaacaacatagttgttctggagaaggcatcttttcccggtaaagccaaagatgcagatctaggctgtgcttgtgactgacagcacagagaggggttcacagccagctggccaagtgccccccgaaagcgcatttcgaatctgctctatttgagagagactgtcttagccttgtttgggaaagtcttcctccttcacttcacctgccacagacttttccaggcaccatctgctgtagtcttggcccagtccctgcaacagttactgctgaaggcacccgggacatgcaagacgggggagcagcctgaggtctggcgtccggcaagcttttcccacttggagccgtctgggagactgtcccggaaacagaagggctgccaacacttggaagtgccaatgtggactgaaagttgaggacaggctccgggctcccccacctcttcctccttgattcattaaaaggaaagaaagaagccacacgaaactctcctgaatttcatttatttctatacaaaagacagagcgtggtcattcatcattcaaattttaacctttttagacaaataataattcctgcttgtgaattcagtgtattttaacaagaataggtctgagggccattggccatgggagacaccgaaggctggctttccttagatttgcagacagtggccctgatggtgcatagggtttcaggtttcctttagacctcagctggctgcctgtgccaccacttagcaatgccattgtctttcctgtgcattttctctgcagagttcgaggaaatccagtcgcgcaggcccctctgcccccatgtccccggcgccctggaatgtgcagtaccagcagcagcgattagaatgggggtctggtttcccggaatgtgcaaggtctcacttctgtttctgctgcctccatgccccagaccagtgctgggccgggctctgggctgcagccatggctgacaagtttccttggaatttaatggagcggggcagacagcatgcagccactcaaactgaaaacttgggaaagagatgtgtgttctggggcagctttgctgcattcgctgggccgtacatgcttctttttcctttccccaggcaacccctcttgcagacaggaggccccatctcctttcgcttcatgcctcattggccattaggaaccttttaaaattggtttctctcctgaccctctgagagaacatagtccaagttccctggaggaagaggaagcgctctgtttctctgcaattcacggctcatttaaatgcagcccacgtgctgtctctccccactcctctgcctgctccccttgtgcttctcatgatcattctcaaatttagtgagaaacctcacaaagggagtttttcttagggaaaagtcatccttggcctcctgaacgtggaccagcccctctccccagctgcacagcatcaggttagttaaccacctgcctccatctgggtcctgtctggacaggcctactcacacctgctgcaggcgtccgacttgccctcaggtgcctgtggctggttcagaggggtggagcccacattccagtcctgacagctaaagttcagcgagaggaccctgcattcagtgtaaagatcaatattccaggtcctctcttcctgccacccagagactggccgtttgcaggcactcggtcccagttgccctgggcctgcagcccttgcattctctctgctttgtctctgctgttgcacccctgccccatcacagatgcaggttgggggaccttccgctgggaagtgagaggctgggaagtaagaggagcactagagggatggttgagctcgcatccagccttgactgcattcgctctcccccacctctctgtaaaggtgctgagctgtgagtggaaccaagtggatgagagtggccccgggcacctgccgataagtttcccggtgtgtcattttctcctgggagtcccatctggatttggttctggatttatttattcagcaagtagcctctttgtagttacttttaatctagccatgctcggggctgaaggggatgccaaagaaatatacgatgagcccctcagacagcataaaggtgaagatgaggcctccagcatgtaccccccaacatataccccaggaaattctgggtgtgactggattttggacctaccaaaagctgctggtgcctggaggatggggccccgaggctggacctcactcctgctgggttactgggctgggaaagtactgatggcagctgaggagtgtgtcccagacttcactgagccattcccaaagattatttcaagttctcctgaccccgcactggaggcctgcggtgctggccttctttatttacagtttctgactggtgtctagcagccttgccagagagagtggcagtgtgtctgcaggcgaccaggagaaatgtcccaggctttagggcaggactgagcatatagcggtgggggcccagcaggcagtctcctggacagttacttctccttgtccttacatggtcgggaggttgctgcctggcttttcaagcgaggatggaacgtgctatccatgggccttaatttccaacttctgcatgatgcattttgtgctcttgcctttgaaaaaacgtttttattttcttgtcactgatgcccaaacccacatggcagaaggaagggaggctgggacaggggaggcgatgagctgccgctgacggacctgcccagtttcttagctcatcccggcctccatcctggtgagcagacactggcccaatccagccatatttttggctgagtttctgtcttcacatctcatcctttccctgggatcctggcaattgttggtactgggttgtattcttatttgtaatctttaaagtaggagtacctttgctggtatttaaagtggaggaaatcaggtgaagagtcacaagtgatttgcaagctgggagagacattagaatgtaaatgtgaggaagcgtcagcatgaggggcttgcctgggctgcacagcttgccttgcctggagcatgcactgttctggcattgcagggaggatggctaccttgcctccctgcaggtgggggactgtgtcagcccctgcggactgctcctgggctcctgggtttgaccagattaaggcagcatctccagtagcaccggagcagctcctgagacgcttttctgtgctaaatctggattttgggtattaaatcaaatgaatttgtaatgcagtcacacattgccctgtgttcagaagggtgccgcacctgttttaatgctctgctattgctcccttgggagtcttaataatttttgaacaaagggccccacatactcatttcgcactgggcactgcatattatgtagctagtcttgaatctaggacagtgcattaaaatgccattgattggatcaatctgctcttacaactgatttgaattttgggaacatgctgttccctgtgaataaaggaggattcatttcttttccctcgaatacactgcgttctgttttccaaattagctctacttatcaactctgctgagaaattggaaggcgggattgttctggctggaagggaaggttagattgttaatcctgcgtcctggccctgatctcacaaagtgtgaagcatgttcccacaatgatgtgggctgcagggggctggaggctggctgagaaggtggggaccaaggagggaggccagcctgggagccagacagatggggtcaggctctcgcttttgccactcgccagctctgaggctttgggcaacatgatttaattctctgatccttgtttttttcatctttctgtagactggtgataagatgcaccctgcaggcttgcaggaaaaattagagataacatttgtgcctattattgggcttgacatatagtagatgctatacaataaataggtcctgttattcttattgataatattattttattgtcaacattgaaggttgggtgggatttgactagctgcgggggaggagaatgagatcatccaggccggaaggaaaagaggcatgaatgcagggggatggggtgaaacactttggaggtgtggggagaggtctgcagggtgggagtgtgcattaaggagttctggggagagtggaggcatcagtgccacatggcaaatgagagggaatcgtgggcccgaggagatggagatggctgtggggatccggcaggaagtttatgtgccccaaagtggcattgtcagttagggggagacactgaagacagaggtgaggcctgcctgaattagcgcagagtggcattcttggaaacttcagaagcttgagaagagccacttggaggtgttgaaatgtacctgggagggatgtggggacctggctctggtctgagagctgggagacggtaacccaggtggccttggccttgaagatggggcatgatatttagtgctttatgtgcagtctcacctaggactcccaagccctgtggagtaggtgatattagctccgtgttacagaaagggagactgaggctcaagcagggacaggcacggtctgaagtcacacagctgtaaggggcagaagtgggcatggaggcattaacttagagccgaaaggtgtgaccttccttagggtggctggccccacggggaatgtgtgtgggttggagtacaatttggtgttcccacccatcccagatgctctgcgtttatgaacccaagtttccacatcagggcaggcgagggcaggaagctctacagggagaagggacaagggacagagccaagaatgggggcagggccccagggtcccgtgcagggacaatgaagggagttggcacacgtgggttagctgctggacagtgtggggagagagctggcctgggagtctaatgggaatgccagggaaagctgccttggtcccctaaagtgaagcccccatgctggccacggagtgttggtgactgagggtccctgctagctgtctggccaaggcagtgtgtcctataggtgtagctctggtgtcctgctggcatggcgtgagtgcccctcatgctgagagccagccctgtgctctggagggaggtggtgggaggaggagggacagtaggaaattgccacctgagcaggaattggcaccttctcccactggcaggtccaggttttatggaatctgaaacttgtacaattcaggatactctcttcaagaaaaaaaaaaaaaacccttaaattatgaatataacattagggatgaaactattatttatatagattgaaaagagaaaatgcccaaaatgacaaacttcagaaaatataccaatactgcaaacatcacaaaatccagaaaaacaagattaaaaaaagctaactgctgaacactccttcatcttgaaaatgtccctgtctcctcctctattttttggctgtgaactctgctcaccttttcacatgacaatgcttttgtaatatttcctaaagagaaaatagaataatttattattacttttattattttttggattattgttatgatcaagtcaatatttttctgctacccacacactcactgtcttctgtacaacctctggcctgcaccaggggaaccagcagggtgagcagtagggtgtccctggagaccacacatatagcaggatagacacagcaatttaactagacacagaagggacttcaaagcacacaaatgtatctcatttaacccaaacaaaatgattatccagttttacttttcccttagcctcttcccccaaatgccggcagccaccctgatgggatagatgtgtgacagagggcaggagaccgtggcctcaaccagctgcagcttcactctttcaattctacatactctctacaagccgtgatgatagcactttgctagggcccctcacagggcagatggagggctccatgctgaagctttgtggatgtttgctgtctatccacttctgctccttgtgcctatgcagggattcaggcccaaccactgcagagagcccaagagcatcaggctcccaaactgtcatggttggtggcacctttagtagttgatacggtttggttgtgtcctcacccaaatctcatcttgaattcctacatgttgtgggagggacctggtgggaggtaattgaatcatgggggcaggtctttcctgcactgttctcatgatagtgaataagtctcccaagatctgatggctttgtaaaggagagtttccctgcacaagctctctctgccttctgccatccatgtaagatgtgacttgctcctccttgccttctgtcatgattgtgaggcttccccagccacgtggaactgtaagtccaattaaacctctttcttttgtaaattgcccagtctcaggtatgtctttatcagcagtgtgaaaatggacgaatacagtagtgcagtcatttcttcatggtcctcagtaaggccaaaaaatacccaacagttccgttgatcaatcagtgaggtccaaacaatttgataagtatttgtgtccctacaacacagtggtcattaaaaaaagacattttaatttcattattcaataagcatgattacttatgaatgggatgtgtgcacctgttgggtgtcacatgacctttcaaatcttggaatcagtttggacaccaccatccccatttccagttcaacactgatttttgtgtggtacattctttttgtcacagtgactgccagaaatccaacttcatatggactcatgaaaagagatgtagcgtgatctgatttcaaaactatgattgatctagagttagtttacaaggtgtctaacagtgatcccgtatcactgtatttccccagaaaacctgaaatatcgatgaattttctgtggtattctggggtcccttggggcagactatgggaaccatggcattagaaccataaggacacgattctggcttcttcctgcctcagatccagtctttacctggcatttttgccttaaagatgaaagcagcatacattttgatgtatctaaagcacatattcggccaggcatggtggctgacacctgtagtcccagcattttgggtgaggcgggcagatcacaaggtcggaagttcgagaccagcctgaccaacatggtgaaaccccgtctctactgaaaatacagaaaatagctgggtgtggtggtgggtgtctgtaatcccagctgctgaggaggctgaggcaggagaatcacttgaacccaggaggcagaggttgcagtgagccgagattgcaccactgcactccagcctgggggacacagccagattctgcctcaaaaaaaaaaaagcacatattccactttgtgcttattcttttgagagaaacacagataaaagtctatcctttaattcatactccccatactgtgattttcatttttactgcaacaaattgtgttaagtgtgataatgaatgtcaaacacttaatgccttgctcttttcagtaacatgaaatattggagaataatgactgaagcttacctgcactgcgtatgtctcttttcttcctccttgaaggaagttgttgaaagttgttaagaagtattatgtgtaaaactctagggatgatgtgctttaaggaagcaacatttatgaagttgtgtgcttgactagtagtttataaagagggaagacgaatcatttattatattgggattgaatcctggcaatttttaaactataaagttacaggaaatgttggctactcttaatgggccatttattgtgttaaatatcagcaatgataaatatttactaggtaagtggaaagatccatctctataagttgttgtaacttaccattttacgaatcttagttactcagtttttctgtttaaaaatgaaatcatgtagcactgtataagtcattcagttttttcttttggagaattactctggattgtctaggctctgtgttctccacatatattttagaaatagtttgtgaatttctacaaaaaatcctgctcggaattttcactgggagtatgcttaatctatgggtcaatttgtgagaaattgatagcttaacaatagcgaatcttctgatccacaagtgtggtatttctctccatttatttaggtcttctttattttgatagcattttgtagctttcagtgtacagatcttgcaaatatcttgttaaatatttccctaattattcgatatttatttttgatgctgttatagttatattttaaaaattttgattccaattattgctaatacatagaaatgcaattatttattgacctgttatcctgtgacattgacaaacacagtcatatattcgtagatttctagaatttttctacatagactatcatatatatcatctgcaaataaagacagttttacattttcctttccaatctcgatgccttttctttctttctcatgcctcattgtgtggtccattactgaacggcagccagttccagctttctgttcaataaaggagcagttaaaagggccaggccttgaccttgctggaggcttcccatcctcattgccttctgcttcctcagttctggcttaacagaacagtgtggggaggaggcatgatccttacctactagggcgttacaatggccttcttcaggttggttgattcatcaggtttaagcgctcacctgggctgcagtcaggctagattatctgctgaccttgccctgtctcctttctgtagtggggtacccttgtaagctagggagaagagatacaggtgaaggccggaaaaaccagcctgccacacagcttccctggatcataccttcgcagtgatatgacgacactgttaggaggagcggaggtggctgagtgggtctccagacacctccctttacctctctgctgtgccactgatgtgtgacgtgcttgcacctatacagagctgccactgagcagcaccgtggccagtcctgtggattttcttctttctaaattgtatgccatggcttgatcaaacatttcatatacagtagatcatgaaatcagcatagaaaacacattgaggtagatggtgttaccacattttatggatgaggggctaacacttggagaagtgaggtaacacgtccaaggccacacagctagtgagcaccatgctgagggtcacactctggtccatctgaggccagagactgtgcacagccttctcctcatgctgagtggcctggacacccccaccctctttcccctgaaccccttggagagtgggcagtggcagaaccaacctgggcccatctatggggattctccattgggattgacccgtctggaaggaagacagttgacccacagttaagatcacagcagatgggccagccagggtttctgtagaacatcaggcagtggccactccatctagtttcatggatgagcctttttaatagaacaggaatctaacactgaaccaagctgcttttagacacacttttattcctcactctgaaatggcatttggacaagccaaatatttcttcttctttcagttgacattttgtccatctttgaacagttagctgatgtttcttctgtttagttatttctgttctattttcctgttgccactggtccacccagggatggtaagaatggaagtcaatggttgctttttcatctgggatgcgtcacgaaggctcagtcaggcttgtcatatggtctgtgctcccactgctccttctttctgtttcctcatctacagaatttggagagtcctggacctgatctcaaatttcacatgttctttatcttcctgcagcacgctggggagagggagagacagggattccatcacagaaggttggagctggagcagacttcacagctcattctagaggcatttggtccatcttcacagctcattctagaggcatttggtccatcttcacagctcattctagaggcatttggtccatcttcacagctcattctagaggcatttggtccatcttacagatgaggaaatggaggctgcccaggggactgaggctggaactgggccttccagtggccaggccagatcctccttggtctcccttgttgctttcctggtgggcagaccctggagccactttctgtgactgtgtgagaaggcgactgcccagcaaaatccatcttcaatccatcttcatttttgcctctggcgtgggcagattctcccatacctaattcgggaagccagaaagaggaagtcagttaatgatccttagtgggaaggtgctagtaatggtccttctcgtgagtttctgaaacaccacgccgtctctgtgttgctggcccggccggagttaaacctcttcttggcctttccccaggaagctggtctgaggaagcccagatgcgtttgtttacagctgtctctggtgacgttcgccaggctctgtgttcagaaggaacatttccattcccttatttacacctcccactggagtgctcgaggagacacaccaattatttccaactacctagaaacctgggagggtagcagatctgtagggggccggtgttgaagcgagaagctgtaaatctggtgacactgtgggcttgggagggcttgcccggatctacctgttacttatactctctattaagaaattttagtgtccatggagaagttatttaaagtctgcgagcctcagtttccccatatataatatgggaaggatacctgattttcctgttccacaggaaggtagaaaaaattaaattaaggcaactgatgaaagggttttgaaagcaaaaataataatatgatactgtcctgaatttgttaaattattcctcctagtagttgcggatctttttctgtaccttagaaaaccatgctatgtaaaaagagatggttccagtctttaaataaagcagctcagaggtcaggggccaggacagaagggggccctttgttcacagatgcgctttcacttctgagaaagcaagtgtgggagaggcaggtggtcctccagatgtccctgtgccccatggtgtcaagttgggttactatggccccttgtgacccagcgtggtagggatgtgggagccagtgggtatggaactgtgatgggtcacaagagggctgggacgtctcacagcttctacttacagcctagagcctggggaagggctgccaccttagtggtaagagaggcatgtatgtgagtgtgtgtgtgtgtgtgtgtgcatttgtatgtatatatgtgtgactctgtgtgtatgtgcacatctgtgagtatatgaattgtgtggaagtgtgtataggtgtttatgtgacagtctgtgtatgagtgtgggtgtatgtgtgtgggtgtgtttatgtgtgtacgtgtgtgggtgtgtatgcatagtgtgtatgtgtgagtttgtgtgtgtgtgcctgtgcatctctgtgtgtatatgcatgtgtgttaggggcaggcacacaggcctgttggtaaatgagacacaaaatacctacaaaatacaaaatatgagacaggaaatacaagccacagttattcatttttcaacgcaacagacataagattaccatgtgaaattgctctgaaagtttccaaaagcttcctgtcaattcgtagagagcagctaacaaaggagtgcgggtccctggagcctgcttgtgcagcattgagctattccaagggggaagaatggggtgcatggctcttagctgcagaccagcctagaagccctccagcctgcttgagcagacttgttaagaggtagcagcaggtggcagagattaggagctggagtagtaggctaagggtgcacttccagggacacactgcctctgccaccacccgtgccaccaaaatgggagcccagaaccctgaatctctagcagcctgtttctgaatcagttaccttgggtgtgcgcctctggtcgacagaaactaacttttagccctcctgggtgagagcctcacatcgggacatgtgacagctttgttgaaagtagctttggaaacgcccaccacgtggggccactcactgtagtataaacggtcatgcaccactgagtgacagggatacgttctgagaaatgcatcgttaggcgatttcatcactgtgggaatgttacagagtgcgcctatcaaacctagatgccatagcccactacacacctaggccagatggtagagcctgttgtttctaggctgcatgcctgtacagtaggttactgtactgaatactgtaggcggttgtaacaatggtgagtatttgcgtatccaaacatagaaaaggtacagtaaaaacaatggcgttatggtccacggttggctgaaatgttatgtggtgcatgactgtaggtataaagcattatggtcgtttgattttcctctttttctcacccacagtcttaaggcacctcttatgccttttgtctgggatgtcccgggcagggttggaacatgtggttaaggcatggtggaaactgctttggggacggacgatggcctcagcttgccttggggtgtcagtgggaaagataggagctgcccctttgccttcatgtttcttcgtaataatctcagatctacccatctggtgagcctctcctagagaaaagccccggtgctccttcgctcctgcggtgtttctcaggagggttgcttctttgtaatggtggggactcagggaagggacgcaggcagagggtgataccacatcacaaagggacccttggctgggtgcggtggctcatgcctataatcctagcactttgagaggctgaggcaggtggatcacctgaggtcaggagttcgagaccagcctggccaacatggtgaaactctgtctctactaaaaatacaaaaattagtcaggcatggtggtgggtgcctgtaatcccagctactcagtaggctgaggcagaagaatcgcttgaacccgggaggtggaggttgcagtgagccaagattgcaccattgcgctccagcctgggcaacagagcgtgactccatctcaaaaagaaaacaaacaaacaaaaacacaaacaaacaacaacaaaaaatacttgggccatcagcttcttggaaaggctggtgtgaggtagaagcatttgctggtgcctctgctcgacaccagagcagaggtgattttttggtgactctgttgagagcagagaacctgagcaaagaggttatcatgagtggattttactgccttacttgggtgggcattcccttgggagttcgatggacatttgcagctgagcccaggcaggggaactgtgctcactccgccttcagaattccaaaggctgagcatgcattttggcttcctctaacccatgtctttctctaggtgaccacagcagagtatcattaagtatctattctttgcttttgttctcagggcaggaagatcccaatagtttgcgccataaatataactttattgcggacgtggtggagaagatcgcccctgccgtggttcatattgaattgtttcgcaagtaaagagagccttcctttttcctataacctctgaagctttcaccgccactagcaaaacatgagagctctttttgagacacattaaagtgtcaaagtgtcactgaatatcttcctactttaagataagtgtgtctcccttcaaacatttgccctattcgactctatgaatctacagtcttaacccttctaaatgtttaaagaacctcgggctctgaagagattccctaagaatattttgtaagtgaaattgtttgatgcatgcaaaaaattggcagattgtttagtttttaaatgttaagcccaatatataaagaagcgattgctaggtgtgtgttgctgttgcagaacccattcattaatcaatgtgttgaagcgttcattttaaggtgttgcaggcttaagtgtgtacttctttggattttaggcttccgttttctaaacgagaggtgccggtggctagtgggtctgggtttattgtgtcggaagatggactgatcgtgacaaatgcccacgtggtgaccaacaagcaccgggtcaaagttgagctgaagaatggtgccacctatgaagccaaaatcaaggatgtggatgagaaagcagacattgcactgatcaaaattgaccaccaggtgagtatgttttcgcctgcagaggtgagttctcagatgccctggaacacccttggcaaaggcaccagagctctctgattgcaggtgattctcagggggcactgaagccagtctaaaccagtcacaggagggccttgaggagatgctgagtatggcctgggcgtgtgggagaggcaggggctcaggagagcttctgtaaggagccagataaaagtttttaaaataatgttttaaatgtttatcaaagaaagcaatagatttgtaaagaaattagtaggtaagttgtgaaaattgagtctccttcccattcccgatcctgtggcaacccttgttacagattttatttatcctccacagatacgtcatgcattcacagtgaacatagaatttactggggtttagactgagccatccttaacttgtcaacagttactctgaaaacaaaccagctctcccaaattggggttttgcagggtaatgaggtgtgtttcagaacaatattccatactttatatatcttggaaaccttgagttaaaacagagctaatggatttcttcttcccagaccttctcagagcttttagtatgctagtgtgcacgtggcttgcctacaaaagggtgttgactgaactatttgcccaaattataatcatttgagtatacagcttttttttggaggggggaggggcagaactgagccataccaagatcaatctggcaaatgctgtatttgaaaatgctttctatttaaatattctctttgcaatcatttttgctgttgaattgcttagcaaagtcttcatgtctgggacaatatccatttcttactgactcatcaaaaacccccactcgacactttgatgagagaggttttatttgctgtgtggcatgttcagtgaaagcgtggtttctagtttcttcacatccttgtaattttctggacttcagacggagggaacaatcagaggaggttggaatcctgcctctggccaaggaaaagaccagagactgagccagttggggtctcttgtccagccctctgcttgcctccctttacctgggtgtgggctgagtaattccagacaagcgtagaattaatcaggctatttgcgctgttggatggcatgctgggtacatctccttctggaaacagctctgcgtgtgctgtttgggtggtaggattctgggtctcctctgtctttttatggcatcaagttgctgcccagcccaggctcctttacggccagtcttcagaaaaccaccagctaacacatttacaaccctccttccccgatgttcctataacctctctatggccgggtggccaggcacggccaaagaggctcagggtagatatagggtctgtgtccggtgtgtgtaactggccttgagtgaggctgcagttgtgtgttatttctattaggtcactgtggaatttctagcaacaactaatctttcaaagtgtgtttattggtcacaggatcattgggccagcctctgccttcgttctttttcacctaatctgcataatagctgtattatccccattttagagaagaagaaacagggactcagagaagtctagtaacctgtctgagaccacacagcaaacacgtcatgaccctgccctcctaaggcagccaggctactgctcccaacgtgtccaagcccatggctattgttggagggatacaggctggccccatggaatgatgggacagcttgaccttaaacagcccatggaaaggtgggtgcatctggtttaggaacaggctgctagaaaggtatccaggatgtggtagtctcaccggaaggagccagtcagaatagcacagcctgtggccacgcgtggggcctgttcagcctcacagagcctttgggaggcagccagcagcagggcatgagctgtgtgcaggcaaggcgctggcctggacgccgcccccactgagtaacttcgtgtttggaatgcgtgggcacataccgtgcagctgcttctggccggcggatattcttttccaattttgagccaaggtggagactgtctcctcgtgtcatccctggcatgtcctggcaagacacgaacgatctcaatagacaagctttgcagagtgtgtctgacctgactcctgctgtcctgggagctgagctcttcagccagcagcatgctgtttgacatgtgtttcaagtcccccaagaaagggtgcttgaaatttaaaattgaactgatgtggcttttctaaatggaattggaaatgaaaggatattaaattgcagacaaccacacaaaagactggtttccactgactaaactgcttttttttgctgatagtagttggaagtagggagagtaacagcatctcttccagctctttctcttttgttcccttgttttgatgatgggttatttcgggggaggctctggctggccttgctttgtgtcaccttagggataacaaagaggatgaaagagatcaggaaaacagagaaggcagaacagaaccagcagaaactgtgcttgaggaatgaaaatcacctacatggctccttgtcgtatgagactgtggcccaacctcccccaaagccacttaagagtaacccagtgaagctggtgagactgcctgccgcgtccatgggcccagtgactagcttggtggcttatcatctggacccagctcctcccctggcatcctgatttcacttggagggtcctccattgtccttcataaacgtgtttactttatttttttttattttttgagacagagttttactgttgcctaggctggagtgcagtggtgcaatctccgctcactgcaacatccacctccagggctcaagtgattttcctgcctcagcctcctgagtgactgggaccacaggcacgcaccaccatgactggctgatttttgtatttttagtagagacagggttttgccatgttggccaggctggtctcaaactcctgacctcaggtgatccacctgcctcagcttcccaaggtgctgggattacaggtgtgagccactgtgcgtggctataaatgtgatattcttgagactttcagtgaaataaaaattaccatggacacctgtggtcattgtccacttgccacccacctaccccccttactggcagcagcagccagcatttcacatctccgtcatcggacagcgtaggtgggcccatcagtcatggtgtcctaccctctggtgccaaggagcggacacatgaccaagttagggcaagcagaggctccccctggaactgcaaagtgaagccggatgtcacccacagagactaacatggtgaagctgctgtaggccctgctcttgagaccccagcactgtctgagttcttgcactttctgagtccagtttcatatctgcttttcctcccgttcttggagctcccctcacatctccagtggcttgaagttgccagagatgtttctgggcttgtgaccaaatgactccttttctgcttctcactgctgagcagacacatgtgcgctcactttgcctgctgagtcttgggacccggaagagctcttgggagacgctcacggagcagccccctcttgccggccctgctgactccctccaagcaggaggggagaagccctggctgggcatcccttaatgtgcttctgcccaaatctgaaactcctctttcctcgggacccacgaccgtggccagcctgcctggggagggaatcccagctgcagaaagtcgggacagtatgcgtgtaaacatgttaatagaaagcagctttgagggcaaactagttcagctttagttacaaactctttccaaatgtgtttgacatgagccactgccagtgtgcagcatatgtcaagctttcatccaatggtggcattttgtcccaacgggtttttttttttcctgagcagtttggggcaggggtggggagagggagagagaaaagtaaaaagagagcagtttggtttcttcaggctggagtacaaggtagagataatgggatgtgttgaagaaagtaggagggaaagttactttagttacagctgtttgtccagctgtgctgattaagaaacttggagaaaagcatctctggaatcatgtccttcccatcttgtatatagcctttgcagatctcctgcggttctgagagagatctgaactgcttaccagggccttgagggccccatctgattgggcaccctccctccctctggccctcctcctcttcccctcctcccctcctttctctgcccccacctgctctgctcagacaccccctgctcggttacttcccacaggccagggctgtcccctggggccttggctgttcccctcctaggagcacccctctccagctcctcatggagccaaccttcccatccttcaggcctctgattaaattctgccttagacatctctccccaccccactgtgggaggtgacgccccatgccccagtctcctcaatcccaccgcgtcactctggggacacatcaccccagggacaactgcattccactcttggtttttccctccttgtctattgatcacaatttagagtcgcctcactcatttctcagtcatttgtcaaatgaagtccatttctgccgctagactgcggggttggggacacatccggctgatcggtcctcaggtaggaggtgcttggcaactttgtcccgagtaggacgttcacagctgtctgccctggaggaagcaagggcacccaccacgtggatggaattgaggggaaggcacccgcggctcctgcatcgagcttccgtcctatattcaatgaggaaatgaccctgcagcaggctggctgcagatgcccctgccatcccgctttgcctgcctggagtttgatggacatgtggtcctgtcagggctgcagcaggtctgtggtctttggtaatgcaaagcgctggggaaacagtgagctttcctgtgggtgcttttctctgacgccaacaaccaggtaaatatttggaaacggccttgttgaggcttgtgaggtggttttcctccctcccctgtaggcctgcgccacccctccaaccccccggccaccttcaggccagatggcacccacagacctgtttgaagtggctggacagggagccctctgggcgctggggccgctgtgtttgcagagggtcctcttactgctgagctggctggtgcagcggggaggccaacacccctgatcctcatcaagttcagaggggagtcaccgcgggtgaggggcctggggccttttacatgtcctgggagctgctgggcaggccgctcttctccaggccaccagaacttggccctgcatgtggcgaatctttcctgagtcagctgagtgaggggggttcaggcagccccccgggacgtggcagtggttggggatgggagtgggctggtgcgtgccatgactcacgccggttctcctcaggcaagctgatggtcagacgtgctgactcagtggcctgagctcgtccaaaagtgaatcagagaacgcagggcctgggctcacccactgccctctcctggagtcatctgtcactcatcctcatgaaggaagcgcctgggagcctggaatgcacttcgcactgccccagctcccctcttgtttctgtgtttttccattttggattctttccccccactccttctgtactgggcattttgtggtctcttctttttctccgagaactctgagggctaccattgcatttgctaatgatgccacagacggtgttgatgttatgaggcttctattactgtattgattgttaccatttttagggggacaggaatcaatatttcatgagggaatgtgaagccagacagtaaagtagaagctggcttttattttgtgccaggctttgtccagaggcgggtggggacgtggctcctcagctcttgactgcagctccttctggcatgggaaacgcttcagttccccaaactctcagagctggagaccctgtgtgttctctggcccggattcaagaacttagttgattgtcaaggaaattctttggctatatttttctcttaatatggtaatggcttttttcactctggcactctcttttcagggaatcggattaagactattatttatggttctgaaaaagcagttcccaagttggtgggactggatttgtttaggaatgtctcctgtcctcttcattgaggggggaatacaaattggttccatttgacagtttatcaagtgtgtgacagagtattagagtccagggttggccaactacagccagtagtccaaagctggccctctatctgttgttgtaaataaagttttattgggacctggtcatgttcacttatttaggtagagtctatggctgctttcattctgcaccagcagagttaaatagttgggatgaagaccacatggcccatgaagtcaaaaatatttgcttcctggccctttataggaaaaaattgccagccccagtggtaggcaatttacaccttgtcctagaggagctgaaagtggctggaggcaggaatgctcataagaaccaagcgaggtgaagcactaggtagctgcggggagcggaagagaagctgattagctgattttgtttgccctttcttttccagagattgtgggtttttttttttttttgcagagatgaagctttggtcttgccacaatagcagagggaggccttatttttgtccatttctctatgacattggtagaaaggagtttgtcagaattccaagctatttggcaattatccaattttgagatcctaatggatctttcaaggtctagtttgttcattcttttagtgattccttattaattccctgattttatacatatgtgttgaacatctgtcttggccaaatacttgttaagtgctgaggatgcagccacagtgggcaaagccatgaggcttaagatctagtgtgggaaacgggtgaagtaaagtaaatatggcaataagtacagtgcatgaagcaaacaggtgaaggggtagaaggcctcgggctgcaaagatagtagatagtgtaagcagggaatcttatctgaggggtgacatctaggctgagatggaaaggacagtgagagccagccaaggaaacaagctgggtgacaagagttgcaggtggagttgcttaatttcccatttctgctcagcctgcagaacctagatcttggactaattgcaaactgtcatttccttgtgagtttattagaaccctccagaacaagtttctggttagctagtttctctgtgtgttgctcatttcttgttggttctggttctttggggttcctactcatactccggaaagctccaatgtcttaagtagtcagtctcccaagagtctgaaagcacaaagattcacaatgatacgatcacctctcagtcatagcagcatcgatgcagttccgtagctggtttcctaaagccatccagacctctttctgtggcaagagagaaataagaccttctggtgaactgaggactaattatcctaataaacatgtgaattaacagttcctttggttaaacaaagcaccagaatctgataatgggaacatgtgactcacggtatttccctctttgctttatctaccaggcagctcacgaaaccactggccttccctgtgttcccattttatgtcataaatatatgtttaattaacttattataaaaggccctttgtcatggaccatatcaaattattcttatatagaagaggttatacatgttttaaacattttaaaataaatctgaaaagaatactacatcctgggcaacttccccgcatatggggctcaaagaagctctatgtggttatgggtaaggcggagtcagagtgccttcagtgtagttcagcagatgctgagaggctgctgtgtgctggactctgatcccactaaatagagtagggctgagcccctgcccaccatgacagcctggagatacaagctgttccctttgcctccctgagccctgagctttatagcctatagacagctgaaaagcaggctgcatcggttaccccgtcagttacccagacccaaatgccaggccttggctaaccccagttattacctaatttcaagatcctaatgtatcttttaagacctggcttgttcattctttcatttatttacttactcattgattttgtaaatatttatggagcatctgccgtgctacatgctgttgtagcagcatcagccaccctgaagttggtggatgaaaggggacagatcaaaggggctgatgtatggaggagacacaagttagacttgaccaagacaatcttattcctcctctggatgccacgaatatatacagtcattagctgttgggcccccatgaagactgttgacattttgtggtttaaacactgaagagtaagggaatgttggaaatggcaaacatctgatatagtgtaaagaagactaaatattttggtggtgttcataaacactgaggaggaaagtcgtttcattttgttcatttgtgtgctctctctctctctctgttatggcacattatcctctgttctccttctccttttctttttccttttttctcccttcatctccccacttctctgatctctcccacctgaaccgcttctaccctgctgccctcccatccatcctacctcctctacttccctccctagacagtagtaatcacatgtcagttggagaaacatgatggcgacttggtcacaccgttcttctcagtctgtatatgttggtgatctccgtgcccatctggtagatccctccttccctggctcttctgctcaccacaaccacccttgactttgtgatcgctgataaccttcaccttctctaatctgaatcccaagcttctcagtcctggcccaccacctcccctcctcatccactccgaaccctgaacggaagctgaatggaaccctgaacggaagggttctgaagctgttgagaaccctgaacggaagctgaaatatcaatgggtcattgcttttcacagtcctctgtgaaagattactggccaagccagcatctggagaattcctggtctaccgcctccctgtctggagaagctggaagagccagctgcatagggcatgtgacccatgtactcacaggccctgtgccctgagctcactgttttaattttatctttgaatttgtatttttgtgaataaagtcctatgagctaatggagcatgctcagagaacttggggatttagttcaggctggattcctcctactgcctccccaatccctggtcccctgagacctccagccccacctgaccttcccttccctgtttctatgcagcgatcattgctaccctccatccctggaaggggtataggcacagggcagttctaggttccaacttgggcaccgcataacatcttagtggtgcagggttcaggctgatgatgccatggtggttctgtgggctactgggcagggtcaagccgctctcaccctgatccaggtacctaatgcaccctgacacagaagtggcagtgtccttggggtcatccattatccatgtgttggaggagtgggcccttagggaagatgcttggctcaacttccccacccctagccagggcacgatccgaggtccaggggttggtgggcacgaggccaagtcgtgaggcctccagtgtctgcactcactgtcccgtaaataaccacaacaataactagcaaaccaaaaccagtgtgataggttgagagagacagaatgtggaagaagggaaaaagctttatattttagtacctttaacagtgctttctgtatgctttatgaacaaggagcctgcatttgcattttgcactgggctctgctaattttgttgctggtcctgctccctagtagcccgagtcagcaaatctttggttcatctgagtccacagtgcattgacccgccctttttcacagttcctcccctgcccatgtgctcacttccctccttacccagcttggctcactctctcaagcaagtctttggatgctgacatcccccctaaacaacccttctgcggcctggtttgattgtccttaggaggcgtgcaagttctatggcactgcttcttgctgggtatagaggatgtgctattttgtccattgcatattttttaaagaaaatgaaaggttagcataactgtttccagaaggcacattgaatcactcagttgagtcccagccagttgctgcagcgttagcctttgaagcaaacttgaaccaacacaggaccagcctggaagtcccagcctccggaaacgatgcagtggattctgcagattcagcaacaaaaatatttttgtaactcaggaacacttcgtaattttcaaaggcgagaaagaagtaattgacttggcttattaggttgaaaaagagttgccaattttttctttggttttgttgttattgttttttgttttttttcttttctccaagcttcagggaatgagattaaatgagcactgaagtgctactaggcagaacctgaatggaaggaagctgaaatactgatgggtcattgcttttcacagtcctctatgaaagattactggccaagccagcatctggagaattctaggaatcccccctcctcttgcagcggtataagtttgcgggaatcatctcaccccactggggagttgtatgaaaaaagggatttattagggaccctgttgcctgtttggatcttaccaatttaactattgtctgctaatggatgttttggaaagcaaccaggttttctgtaaagaacagctaattgtcagagctgagatgaccatgggagatcactgggctcaactcctaattttagaggtggtaaaaccgcaacccagagaagctgatcaagtgggccaaggtcgtagactgagttcatacaggaccaagacccagccctgatgtcctgctatctgggacagtgttctcccggcacacgtggagcctgagggggtaatgtgtgtgtgtgtgtgtgtgtgtgtgtgtgtgtatgtacatgtactcatatacacataggtgttttgcctaggttttcacttctgccccaccttggttgatcttggagaatgagcccgaggcgcaggtgcgctgtcagcctgggggcttcactcagcacaggcccaacttttctgctctgggggagttccagcagttatggttcatctgtggttcagttatggaacccacaccacacgtagcgcccccaaagccgaggctgcatgcacagacctcccctcccttctcgtggtgggcccctgcttggattcttcccaaacttctcctttgccctgctctgtgttatacccactctggtcccctgtccctgtggagtgatccagggcacaaggacagctgtttcactgctggccgctgtgtaccccgagcatctgggaggtggggagcgggctggggagaagaacacctggagcggaggttgggatcagggagggccgcagtcccggtaccaccaccacctgctgtgggacctgcagtctcctcatcagcagaacagctgtgaagccatcctgcccgtccacagggtggtgggttgtgaaggctgcatacctggcagagctggagaagctctggggagatgctggacatgcacgctaggagtggtttccctgccttgcccagactctgctcccatcacctgaacctccctgtcaccaccacggaactgctgtgaccattgctttcttcttaagcagattaacagacatctcctgccccaccccgccaaacaaacaaatgaacaaacaaaaaacgtgcttgaaggagtatgaacttatacagtcttttctaaacactgttaagtgctggtattgggatcttcttttaaaatgaaccatattccccaggctttggatgacactcatggttgcccaccctccaacttccttccctgctggcagaaccctgggtttgttttcgttccacccccgaccccactgcattcctgactcaggcaaatctgcagggtccagtgcagtcagggggccacgttccctcctccaacgggtgctgaggtcgctgcttgattggatgctgctgatgacctgcgaggaggagggcgccagggcacttttgggactttgctcttctgaagagatgcttccacagcacggtcgcagtcacgtcttgatgtgatgtctggaatggtggtggccgtcttgtggctgtgagaacaggctgaggttgattggatggaaggaaggaaggatccttgttcttgacactgtctgtgagccttcaggttatcgccctggcaccacccagcccttggagtagacacctgtctactctacatactccatttggagttgggttttttggtcacttgcagttgaaagcaccctaactgatatacacaaactatttttagtgcgggtctgtgtttggcccttatggaagactttgggctgagctgcccatggtgagggagacggacttcgtgtcttcttaccactctgtgtcctggtggcttgtatgtgtctctgcccatgaggcaaaagcctaaagggcaagggcggattttcttaatcggatgttccttgcaccaagcacataggagacactcaacgaatggttgttgagagagttctctttcacggaggtggtgttttgtgaaacgatgctgccaggcctgcttgttatttgtctgttggttgtaatctgcatgaatgcaaagagccatctttaatcatgctgtggaccagcctcttccaaggtattagcatgactcccactacctgctcagcatcctgcctatggctaggactttgtaatttacatagatacgctggggagacagggagcccatgaccaggactctgacaccctcactggagctgtttctacatctaccctgggtggctgtctaggacattaggcgattcgtgtcttcctaaagtccctctgttgagagacttctggctctgttgagaggacactatttagcattgtgagtccctgcaggctgggggccagtgggcattttccttctagatgtcccctctcttcttctggcctcccaggcttcctgctcctgagactgtgagaactggcctgtgctgggctcactgcagaaagaccgtcgtctccaaaggtcttgtgccaaacttgagctacaagctctttagccgggcctgaggtctccgcctgggctctgggagagcagcagtggctgtttctgccccctcactgctgtcatgcccacacttcacttgcattttcttcgccccccagccgtgtgagaatctggtatgaggagtgggactcacgtgccctctttcttctcctcttccccttggccttttcatctgtcagtggaggacagatgtttgccccgtttacttctaggctcactgtggggctccagggagatggtgaagtggccaaggagaggagctgccaccttcaagacggcctgtggccagtgctgctttaaagggagactcagagatgctttgctgtgggtggcgcgggaaccagcatggggacagcagtgcagaggccttggactcagagtgcgtgggccccacggggcttcacggcgcctgtggctgtgcacttccagccttatctgtgctgcatctcctccacattcccctgtggagctgatgtctagacagctatggaattaaatgctcaattaccgagtaggaatttggccagcagaggtatagctgcggagcagacagactcgaggtgaggctcacggctgagaacgggccccacctggctctggaatgagctgaggggccccatgctcctgcagccagtggctcctgtggggagttggggcagtgacccccaaaaggcagtttgacctcatggagagccataaatctggcctggtcaccatctctgcaacacatcattccattgcaaagatttctgcctgtgattggaattctgggtgaacgtgtactgggcatgtgggtctgagagctgggaagcctgttctcttgtttagccaggctgcccatgggctgtgaggagtgcccccatctctgagcctcagtttccacatctttaaaatggggggaaaatacagctcaactcctaagggtgccgtgaaagtactttgtcacctgccaggcaaaggctcattcctttcacagaaatgcaaggtttacaatgtgagacccctccctacttcgccgcatgtgtccgcttgcttttttctgtcttagggttgccctacatgagctaggaaatgtctgagtgaataaaaacgtaaacgagatgatcactggtggtgcccattggtgcagcctttgcctaaatggccactacgtagccacattttctcgtctgtgttcaggtgaggactggttcctggggagactccctgggttcacattatgggtgtctatcttgtcgaagcccatatggtcacccaagtgtgactgaaccatggggtgctctgggccccatttttggcagcaggcagcatcccctggaggcctggccctccccaggagcatggagagcagtgcccatggacaagcagtctgcagcctccatctcctcctccctgcccggggggctcccccgccccagcctcgcagcttctccaaaagtgtttgtctccttgccgcatcctctgggcctgagctcagatggtggaaaagaagagctggaaggagagttgcctttcggtctgtctgccttctgaggtctcctgagacatacaggctgggcctgcctccctttctaggaggcgccgatgggtggtaaggataggggataagtgagatgtgaatgaggatcaccacagcaagccctgactcataactttttgatgggttttcaatgtgtggtgaagcaggcgcctgctgggcccccttcctgagttgagcttgatctcctgcctcctgtctgtctccttaggcagccaggctaccctgctccagcaacctgtgccaccccgtccctttacctgtcccaagcccagccccgaaggcctcaaaggcctggccttccagccagtccagggcctgaagggatggcagtgtccctggtggacctcccccagcatggcgtagcgcacatcccagccctgcctcctgccccgcctgcacgccatgaatgctgaagtcatgcctggcaggggctgctggcccgggcccagagtaaacaggctgcgctgagcttgctggtgtgctgctggatgctgatgagcttgaggagtgtgggaagtcagtgtggggccgagtagggatgctgcaggcctgcatctccccccagctgccctgcacgctccagcctcaggcaaccccacagggaaagggtcacccactgtcagggcagacctttaccatggctgggtgacatgggctggctgtgggaaggtggttggtggttccccctgttggatttgcacaggcccagatgctcacagcaaaactaacacctagatgatgcttataggagccagcgggtaatcaaagagctgttcagatcttcatttgcttcgttctcacagtggaccattgaggtagctgtatgttagtcccattttccagatgggaaaactgaggacctgagtggtcgtaagctcaggcccctatctaaatcacacagcctggccccaggtctatgctcttgaccatggacagtgctctcctggtcctcttggtatctgtgatctgagggaccttcctcctcctcagtcttgtatagtcagttttaggtcttagactctttcttcacatccctttcttctttcgggagctctctcacccagcaccttccttatctagtatgtgttgggggatatttgtggcatgatgtggcgctgtgtagtggatgagagagtctgtttttccggtttcagccccaggtttcaatccctgctctgtctcaagtcacccagactcttggaggctcagtttcctcatctgttaaatgggcatggtggtcacctcacctcatcagctggtgtctgctccatccctggtggaggagatggctcaagtaaccccttggttccacctgccccaccccactggtcccctggctctttcttttttgagatagacaaacgtgaggctctggatttgcagttcccacgagggctggggtggctgtctgctttctgggtctggtccatgttttccagggcagctgctcgttctaagtgaacaaaggctgaaggaactcaggaggtttgctcggctccgaggatggcagagagggaaggggtgccgatgccttccctgatagagctggggaggcccttctgtggttccccccagctccttggcttgggtgaccctggagctgacttctgttccattttgttgtgcagagttgtttggggctcctggctctgcctggcctttgtgggccactggagatcagggcttctggagttggccaattagcccgcccagcccagggagcacaggtgtctgatggagggccttttcaggagaggagagatggcccgcctgttgggtcttgctgtcttgggtcctggaggccttgctgtccccatgctccatccatgcccttgaccaatgtggccctgtactcagcataggcatgcacctgagtcagtgcaattccctgtccacagagcaccccaaatattccaggcctcaggatgggtgtgcacatgatgagccgggcaggtttcaccacctgtagcttgggatccttcccggggcttggttctcgaaggctgccccaggcagtcacaccccaaaccctaaattcatgttgtcttcctctgtctcttggcctcaaggtttcagagtgagtctgtgctgatagcttcaagatgtgatgagaccccgacttggcctccagttccctccccacggtttccttggcgtgtgtgcggcttcagtggtcactggctcccacacagcttgtaatgtgtggattacgggtgggagggaagtccggtcctgcctgcagcaaagggatgttagtcgtgagctcagttccccatcgggcctggtgtttccaaatggcccggcactgtccctgcttggttttccatgatatctgtgcctttacccatttggttaaattaaacaaattcagcaatgccagccattgtggtttcagggtaagttgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccggaactcagacatggactacatccagaccgacgccatcatcaacgtgagcctctgtccctctgcgggtggggcttggggcagggttttgccagaggagaggagtcagcatcggtctctgacttccttgtagtctgggtgaaaggatggaactagaccaagccatgtggatcctagtgccagcagcacgacaggggtcacacggcggggacagtgacactggagcaggtggacagccagcctcctcccaggaggaagaagttgtgttgggtgctttagggtgattgcagttggcttctgggcttcagagagaaaatctccccatttacggcacctctaaaactttctgaaaattgttaaggtcatttttttccagcaaaatattaggttaatgggaatgaatctcagagaagaatcatgccccacactgtagacaccatgctcaggagacggccaggcagggacatagattggaccacgttatgacacaatttgtaacctttccatttctgtttaattgcagtatggaaactcgggaggcccgttagtaaacctggtaacgtattttaaacgttatgtcgtttgtttttatttatgtacacactgtttttgttttgttttgttttttgatgtagggggtcttttcaaacataagcttgccaaagcgtgttatcaagtttctttaaaatgagctctgtgaatgtactgcatgcttgcaaatgaccctatggatcttttctggaaagagtaaggcaggctggaggtgagggttggaaatgttatgccagagagcacacttgtgtctcagagttacaggtaaacacagtgaaattcagggccaatgcaggagtaaggtgaaggtcaccaaaagtgctggccggtcactgaaagagcctcctccaaattaaatctcctgggctgctgaaggagctggctgggctcatacacactttctcttggccaggaatcctcccttaaggcctggctggaatgaggaggagttacccacccacaaagatatcatttaagtctacccttaaatacttgagcagaaaaagtgaagccttagaacatagaccatcagcgctagagggcagctccggggccgttcatagagggcagctccggggccatttgtaggggccgtctttagtaaggccttggcatcaggtactgacatcccagcactcgtgggaagtgcgcacggggcgatgtatccccgcttggcagctttcccttcccagcagaggggcagctgtgctcccagctctgccctccgcctcccccgcagcaccctggggatggagtggagacggctttgcgggtaatgaagcatgacagccctaagctctagggttgttccccctcaagtcagcagagtcatcttaagatcattagaaatgagagaagcaggaaggtgtaggcagccacctagaggactctgagcctttggaaacgtattccttgtgaaacaggagcaaataatatcgtgcattttgaaactatctgtgcttaccgcgaggtgagcacccagtggcgacctggagtgtgtgcgattcttccacagctgcgcgtggcctacgctgcctgggtgtcctgatgcctctctccctgctcccccggggatcccctccatgcagctccccgcttcaatctctgaaatagctcagtgacttctttcatgcacattctctttgggggtgtacctgccggtaagccttcacgattcagcaagccgtgtccttcttgcctttcaggacggtgaagtgattggaattaacactttgaaagtgacagctggaatctcctttgcaatcccatctgataagattaaaaagtttctcaccgagtcccatgaccgacaggccaaaggtaggcaaggcccacatagccccggggactccggagattcggcctgaagctcaactgccctttgggaattggggaagggaaaagtggcagcccctaagactagccaagccgtcttcgatccagaagtgaacaggaatgcacattactaaatccctggtagaaggtcacagacattgcgccatttttgtcctccgatcatgacaatgtcacttgagtcagtctaatatgtaccagacacgatcctaggtgatttctgtccattatttcactttatttatgtatgttacttaattcttttgccctatcagttaggaattactagtcccattttgctgatgagaaaacaggttcagggagatcattctacaaacatttattgcctaagtcaagcagggagcttggcagtagactgcccaactggagcctggggctccgctgaggcctttgccggtgtgtgtttatgttctgttgggggatgggaaggctgacagtaaataatcagacacattagatactattagtgctcccaagaaaacagatcagggtggctggcaagggagtgactggacaggcagttggtagagatggtgtggccaggaaatgcctcccaaactgaggtctgagtgaggaggagccagcaggtagggatgtggggggaacactccagaaggaaagacagaggactcagcatagctgagtgagcacaaggcccctggagtggcctgggggccggagcacagtgacagcatggaggtctctggggtggaaagctcgccaaggccaagcaagcaggctcacagcgggccatggtgaggggcctgggttgcatcctaaccgcatttaagaacagggaagttcatgatctgattgatgtcactgaaaggacactctgatggttggggggagtctgctggaggagttgctggaagttggggaccggagaaggagctctcccagtcatctggatgagacacgctgggggctcagacaagggtggtggcagtggaggtgggacagaggggtcacattccaggtatacatgggggtagcgcaagcctggggaagggccagctgtcaggatgaggccatgaggaattgaggatcatgcccaggtatctgaccattaactgaacgatgagactttcctgaggtcccccagaggggaggggtccaaaccaggattcgagccgcaacctccgtgtgcccttctgtggcccttcctgcaacctgggggattgggcccccagcccctggtgtccccagcatacccaccaactgggctgaccttctgccgtccctttgttgtctcaccaggaaaagccatcaccaagaagaagtatattggtatccgaatgatgtcactcacgtccaggtgggcaaacaggatgcgtgtgtgtgtcttaaattttaataaacctgaacttcagaaggtgctcacgggcacccctgaaagagaaagcttatgcagccttaagacatctcagtttctgcttataatgaagtagcatcaggaaagaggacaggtcatcagccgtggcccctttgtttggttttatcctgtgtttctgcattctgagctggttttcttcattggcggctggccctccagtgtagaaggttctgccctcctctttgaaggcaggcctgagcagtgcgtgtgtggtggggctgttgattcattctggctcatgtcttccttaccccatattctgttgaaacccacattccaggagggccccaagcccctcccacagctctaggcactctgctttcattgctctgctctgcggcagctcgtgggccgtggctgcaggaatgccagggcaggcccagtgcagggaagtgaatgactgatgtgcttgttttccccgagctggtggaactgcggcctgtggttggcaggctcacggcatcctggtgttttaacctggatgaaaaattctggtgtaatctcgtgagtcctggtagtatagactcaactggcgtggctgaaactgtcagaggtaaagtaggaaaagactagaatatactaacaggtagattaatgtgttcattactatgatgaattaatgattcactcactgtgaaagtattaatatattttgatacacattatgaatgatggtccctttcttcgcactccagaagatggagccacttgtcaaggttaaagtgtctcctcagttgtttgcctttggaactagaaggtggagggaaagatgggaggcccttggcgcccagctccctgggttcctgttccagctctgatacttcctgccttgtgaccttgggaacgatatgacccctgagtgcctcagtttcctcctcttcaggatggggatgacagcgcaggtgcttctggtggtagcggtgatcaccagccacagtgatgccagtcactatctaggccgggtgctttactggggtgacctcatctgatcctcacaactcatattgtagggtactgttattatccccgtttcgcaggtgaggaaatgaaggcacagagaggttaagcaaccgtctggggtcacgcagctagcaaatagcagagctagggctacaaaccaggccaaccactatactttacggactccttagtaatagctactgttaattaagaaataataacaatgatgatggctgcgcattgctggctcacacctgtaatcccagcactttgggaggctgaggcgggcagatcagttgaggccaggagttggagatcagcctggccaatttgtgaaaccctgtctctactaaaaatatgaaaaatttagctgggcttggtggcaggcacctgtaatctcagctactcgggtggctgaggcaggagaattgcttgaacccaggaaatagaggttgcagtgaactgagatcgtgccattgcactccagcctgggtgatagagcaagactctgtctcaaaaaaaaaaaaaaagaaaagaaaagaaaagaaataataataatgatgaaagcactttccttgctgttaccaagtaaatctttgactctggtagagaggcaattttaaaataggatcagaactcctggaggaattttacattagacccagggagaagaagggaactggtgagagcttgagttttgcctggggaaggactggtgtctcttcacactaacacgggtgctttttctctggagcagcaaagccaaagagctgaaggaccggcaccgggacttcccagacgtgatctcaggagcgtatatcattgaagtaattcctgataccccagcagaagcgtgagttagagtcattttcccttattttcccttttcctaatattcttgttgctcctgtaggggtagcaggaagagggagcgctgttccttttctactggctcagatgacagtgttgatccttgacagatgtggtcggacgttgctggtcattcctgctggccaggccttctgacctggctcggcttgggactcatccataggagggtgccttctgtcttcaaaagtccttgctccactaggaccctccagatggacagagcaatagcagactcataatgagtctctggctggccagagagagggtttcaggaacagtgtccccaagccctcacgtggtggtcctgttctaggcttcgggacccttctcctcctggagtcttccagattgtctctgacagttaggcccatacctgtcaacacctccagaaaaataacccaagtgatatcaaagtaacatgacaagaagtagctcaaccatccatcagggtttgttacctgtattggaatatccagaaaaaagtgctagaccaggggccagcaattgtgccctggggctggatctggcccactgcctgcttttatatggagctgtggactaagaataatttttgcattttatttctatttttacttattttttaaattttttattttcataggttttgggggaacaggttgtatttggttacatgaataagttctttggtggtgatttgtgagattttggtgcacccatcacccaagcagtatacactgaacccaatttgtagtcttttatccctcacccctgtcccagcctttcccattgagtccccagagtccattgtataattcttatgcctttgtatcctcatagtttagctcccacttatgagtgagaacatttaaatagttgaaaaaatcctgaaataagaatagcattttgtgacttgttatatttgtatgcaattcaaatttcagcgtccactgaaatttggtttatgacatctttggtggcttttgtgctggagcagccgagttgagtagcttcaacagagaccatatatacggcaaagcctaaaatatttcctatggacctctttacagaaaaagtttgcagacccttatgctggcccatatgaaggaccatgacagcgttttgacgctgacctatataagagctacagttatagtggcaaccacacaaaggaagtgcctcttaacagaagcattctgcccacccttgtaggaactgcattctgagttgcaataccctttataagcaagttggccatggtcacgctacatggcagatagtacctggtacatccttccccactttggggtcaatcttgacctttgatctccttggggtcataaggccatacaagtgttagtaggcatttctagagtggacataatggatgagttagcctaaaaatctcaaaaggagcccagcatcatggcacctgcttgtaatcccagctattcaggaggctggagcagaaggatcccttgagcccaggagttcaagactagcttgggcaacaaatgagaccccatctcaaagaaaaaaaaaaaggtgggggaagaacattataataataataataataataataaaaaccttgataagtatccagtctaccaatggtttattttttattttattattattattttttgagatggaatctcactctgttgcccaggctggagtgcagtggcaaaatcttggcttactgcaacctccacctcctgggttcaagtgaatctcttgcctcagcctctgagtagctgggattacaggtgcccaccaccaaacctggctcttttgttttgtaattttagtagaaccagggctttgccatgttggccaggctggtcttgaactcctgacctcaggtcatccacctgcctcagcctcccaaagtgctaggattacaggcatgagccactgtgcccggcccactgatggtttgaattattctaagttcaccaccatccaatcctgtttgctctgggcttttaggttctaaactgtgcctctgtccatgtaaagtcagatcaggaggaatggagacatgaaacattgctattgtgtttccctttgtgttgcagtggtggtctcaaggaaaacgacgtcataatcagtatcaatggacagtcggtggtctccgccaatgacgtcagcgatgtcattaaaagggaaagcaccctgaacatggtggtccgtaggggtaacgaagacatcatgatcacagtgattcccgaagaaattgacccataggcagaggcatgagctggacttcatgtttccctcaaagactctcccgtggatgacggatgaggactctgggctgctggaataggacactcaagacttttgaccgccattttgtttgttcagtggagactccctggccaacagaatccttcttgatagtttgcaggcaaaacaaatgtaatgctgcagatccgcaggcagaagctctgcccttctgtatcctatgtatgcagtgtgctttttcttgccagcttggtccattcttgcttagacagccagcatttgtctcctcctttaactgagtcatcatcttagaccaactaatgcagtcgatacaatgcgtagatagaagaagccccacgggagccgggatgggacggggcgcgtttgtgcttttctccaagtcagcacccaaaggtcaatgcacagagaccccgggtgggtgaacactggcttctgaaatggccagagttgactcttttaggaatctctttggaactgggagcacgatgactctgagtttgagctattaaagtacttcttacacattg

SEQ ID NO: 5 - DNA, 20 bp, artificial
  Oligonucleotide nucleobase sequence motif
agttaaaggaggagacaaat

SEQ ID NO: 6 - DNA, 20 bp, artificial
  Oligonucleotide nucleobase sequence motif
tcagttaaaggaggagacaa

SEQ ID NO: 7 - DNA, 20 bp, artificial
  Oligonucleotide nucleobase sequence motif
ctcagttaaaggaggagaca

SEQ ID NO: 8 - DNA, 19 bp, artificial
  Oligonucleotide nucleobase sequence motif
ctcagttaaaggaggagac

SEQ ID NO: 9 - DNA, 20 bp, artificial
  Oligonucleotide nucleobase sequence motif
actcagttaaaggaggagac

SEQ ID NO: 10 - DNA, 19 bp, artificial
  Oligonucleotide nucleobase sequence motif
actcagttaaaggaggaga

SEQ ID NO: 11 - DNA, 18 bp, artificial
  Oligonucleotide nucleobase sequence motif
actcagttaaaggaggag

SEQ ID NO: 12 - DNA, 20 bp, artificial
  Oligonucleotide nucleobase sequence motif
gatgactcagttaaaggagg

SEQ ID NO: 13 - DNA, 20 bp, artificial
  Oligonucleotide nucleobase sequence motif
atgatgactcagttaaagga

SEQ ID NO: 14 - DNA, 18 bp, artificial
  Oligonucleotide nucleobase sequence motif
tgatgactcagttaaagg

SEQ ID NO: 15 - DNA, 20 bp, artificial
  Oligonucleotide nucleobase sequence motif
gatgatgactcagttaaagg

SEQ ID NO: 16 - DNA, 19 bp, artificial
  Oligonucleotide nucleobase sequence motif
gatgatgactcagttaaag

SEQ ID NO: 17 - DNA, 19 bp, artificial
  Oligonucleotide nucleobase sequence motif
tatcgactgcattagttgg

SEQ ID NO: 18 - DNA, 20 bp, artificial
  Oligonucleotide nucleobase sequence motif
gtatcgactgcattagttgg

SEQ ID NO: 19 - DNA, 16 bp, artificial
  Oligonucleotide nucleobase sequence motif
tcgactgcattagttg

SEQ ID NO: 20 - DNA, 16 bp, artificial
  Oligonucleotide nucleobase sequence motif
tcgactgcattagttg

SEQ ID NO: 21 - DNA, 16 bp, artificial
  Oligonucleotide nucleobase sequence motif
tcgactgcattagttg

SEQ ID NO: 22 - DNA, 18 bp, artificial
  Oligonucleotide nucleobase sequence motif
tatcgactgcattagttg

SEQ ID NO: 23 - DNA, 19 bp, artificial
  Oligonucleotide nucleobase sequence motif
gtatcgactgcattagttg

SEQ ID NO: 24 - DNA, 20 bp, artificial
  Oligonucleotide nucleobase sequence motif
tgtatcgactgcattagttg

SEQ ID NO: 25 - DNA, 16 bp, artificial
  Oligonucleotide nucleobase sequence motif
atcgactgcattagtt

SEQ ID NO: 26 - DNA, 16 bp, artificial
  Oligonucleotide nucleobase sequence motif
atcgactgcattagtt

SEQ ID NO: 27 - DNA, 16 bp, artificial
  Oligonucleotide nucleobase sequence motif
atcgactgcattagtt

SEQ ID NO: 28 - DNA, 17 bp, artificial
  Oligonucleotide nucleobase sequence motif
tatcgactgcattagtt

SEQ ID NO: 29 - DNA, 18 bp, artificial
  Oligonucleotide nucleobase sequence motif
gtatcgactgcattagtt

SEQ ID NO: 30 - DNA, 19 bp, artificial
  Oligonucleotide nucleobase sequence motif
tgtatcgactgcattagtt

SEQ ID NO: 31 - DNA, 20 bp, artificial
  Oligonucleotide nucleobase sequence motif
ttgtatcgactgcattagtt

SEQ ID NO: 32 - DNA, 16 bp, artificial
  Oligonucleotide nucleobase sequence motif
tatcgactgcattagt

SEQ ID NO: 33 - DNA, 16 bp, artificial
  Oligonucleotide nucleobase sequence motif
tatcgactgcattagt

SEQ ID NO: 34 - DNA, 17 bp, artificial
  Oligonucleotide nucleobase sequence motif
gtatcgactgcattagt

SEQ ID NO: 35 - DNA, 18 bp, artificial
  Oligonucleotide nucleobase sequence motif
tgtatcgactgcattagt

SEQ ID NO: 36 - DNA, 16 bp, artificial
  Oligonucleotide nucleobase sequence motif
gtatcgactgcattag

SEQ ID NO: 37 - DNA, 16 bp, artificial
  Oligonucleotide nucleobase sequence motif
gtatcgactgcattag

SEQ ID NO: 38 - DNA, 16 bp, artificial
  Oligonucleotide nucleobase sequence motif
gtatcgactgcattag

SEQ ID NO: 39 - DNA, 17 bp, artificial
  Oligonucleotide nucleobase sequence motif
tgtatcgactgcattag

SEQ ID NO: 40 - DNA, 18 bp, artificial
  Oligonucleotide nucleobase sequence motif
ttgtatcgactgcattag

SEQ ID NO: 41 - DNA, 19 bp, artificial
  Oligonucleotide nucleobase sequence motif
attgtatcgactgcattag

SEQ ID NO: 42 - DNA, 16 bp, artificial
  Oligonucleotide nucleobase sequence motif
tgtatcgactgcatta

SEQ ID NO: 43 - DNA, 16 bp, artificial
  Oligonucleotide nucleobase sequence motif
tgtatcgactgcatta

SEQ ID NO: 44 - DNA, 18 bp, artificial
  Oligonucleotide nucleobase sequence motif
attgtatcgactgcatta

SEQ ID NO: 45 - DNA, 16 bp, artificial
  Oligonucleotide nucleobase sequence motif
ttgtatcgactgcatt

SEQ ID NO: 46 - DNA, 16 bp, artificial
  Oligonucleotide nucleobase sequence motif
ttgtatcgactgcatt

SEQ ID NO: 47 - DNA, 16 bp, artificial
  Oligonucleotide nucleobase sequence motif
attgtatcgactgcat

SEQ ID NO: 48 - DNA, 16 bp, artificial
  Oligonucleotide nucleobase sequence motif
attgtatcgactgcat

SEQ ID NO: 49 - DNA, 16 bp, artificial
  Oligonucleotide nucleobase sequence motif
attgtatcgactgcat

SEQ ID NO: 50 - DNA, 16 bp, artificial
  Oligonucleotide nucleobase sequence motif
acgcattgtatcgact

SEQ ID NO: 51 - DNA, 16 bp, artificial
  Oligonucleotide nucleobase sequence motif
acgcattgtatcgact

SEQ ID NO: 52 - DNA, 16 bp, artificial
  Oligonucleotide nucleobase sequence motif
tacgcattgtatcgac

SEQ ID NO: 53 - DNA, 16 bp, artificial
  Oligonucleotide nucleobase sequence motif
tacgcattgtatcgac

SEQ ID NO: 54 - DNA, 17 bp, artificial
  Oligonucleotide nucleobase sequence motif
ctacgcattgtatcgac

SEQ ID NO: 55 - DNA, 18 bp, artificial
  Oligonucleotide nucleobase sequence motif
tctacgcattgtatcgac

SEQ ID NO: 56 - DNA, 19 bp, artificial
  Oligonucleotide nucleobase sequence motif
atctacgcattgtatcgac

SEQ ID NO: 57 - DNA, 20 bp, artificial
  Oligonucleotide nucleobase sequence motif
tatctacgcattgtatcgac

SEQ ID NO: 58 - DNA, 16 bp, artificial
  Oligonucleotide nucleobase sequence motif
ctacgcattgtatcga

SEQ ID NO: 59 - DNA, 16 bp, artificial
  Oligonucleotide nucleobase sequence motif
ctacgcattgtatcga

SEQ ID NO: 60 - DNA, 19 bp, artificial
  Oligonucleotide nucleobase sequence motif
tatctacgcattgtatcga

SEQ ID NO: 61 - DNA, 16 bp, artificial
  Oligonucleotide nucleobase sequence motif
tctacgcattgtatcg

SEQ ID NO: 62 - DNA, 16 bp, artificial
  Oligonucleotide nucleobase sequence motif
tctacgcattgtatcg

SEQ ID NO: 63 - DNA, 16 bp, artificial
  Oligonucleotide nucleobase sequence motif
tctacgcattgtatcg

SEQ ID NO: 64 - DNA, 17 bp, artificial
  Oligonucleotide nucleobase sequence motif
atctacgcattgtatcg

SEQ ID NO: 65 - DNA, 18 bp, artificial
  Oligonucleotide nucleobase sequence motif
tatctacgcattgtatcg

SEQ ID NO: 66 - DNA, 20 bp, artificial
  Oligonucleotide nucleobase sequence motif
tctatctacgcattgtatcg

SEQ ID NO: 67 - DNA, 16 bp, artificial
  Oligonucleotide nucleobase sequence motif
atctacgcattgtatc

SEQ ID NO: 68 - DNA, 16 bp, artificial
  Oligonucleotide nucleobase sequence motif
atctacgcattgtatc

SEQ ID NO: 69 - DNA, 17 bp, artificial
  Oligonucleotide nucleobase sequence motif
tatctacgcattgtatc

SEQ ID NO: 70 - DNA, 18 bp, artificial
  Oligonucleotide nucleobase sequence motif
ctatctacgcattgtatc

SEQ ID NO: 71 - DNA, 19 bp, artificial
  Oligonucleotide nucleobase sequence motif
tctatctacgcattgtatc

SEQ ID NO: 72 - DNA, 20 bp, artificial
  Oligonucleotide nucleobase sequence motif
ttctatctacgcattgtatc

SEQ ID NO: 73 - DNA, 16 bp, artificial
  Oligonucleotide nucleobase sequence motif
tatctacgcattgtat

SEQ ID NO: 74 - DNA, 16 bp, artificial
  Oligonucleotide nucleobase sequence motif
tatctacgcattgtat

SEQ ID NO: 75 - DNA, 17 bp, artificial
  Oligonucleotide nucleobase sequence motif
ctatctacgcattgtat

SEQ ID NO: 76 - DNA, 18 bp, artificial
  Oligonucleotide nucleobase sequence motif
tctatctacgcattgtat

SEQ ID NO: 77 - DNA, 19 bp, artificial
  Oligonucleotide nucleobase sequence motif
ttctatctacgcattgtat

SEQ ID NO: 78 - DNA, 16 bp, artificial
  Oligonucleotide nucleobase sequence motif
ctatctacgcattgta

SEQ ID NO: 79 - DNA, 16 bp, artificial
  Oligonucleotide nucleobase sequence motif
ctatctacgcattgta

SEQ ID NO: 80 - DNA, 17 bp, artificial
  Oligonucleotide nucleobase sequence motif
tctatctacgcattgta

SEQ ID NO: 81 - DNA, 18 bp, artificial
  Oligonucleotide nucleobase sequence motif
ttctatctacgcattgta

SEQ ID NO: 82 - DNA, 17 bp, artificial
  Oligonucleotide nucleobase sequence motif
ttctatctacgcattgt

SEQ ID NO: 83 - DNA, 19 bp, artificial
  Oligonucleotide nucleobase sequence motif
tcttctatctacgcattgt

SEQ ID NO: 84 - DNA, 20 bp, artificial
  Oligonucleotide nucleobase sequence motif
ttcttctatctacgcattgt

SEQ ID NO: 85 - DNA, 19 bp, artificial
  Oligonucleotide nucleobase sequence motif
ttcttctatctacgcattg

SEQ ID NO: 86 - DNA, 16 bp, artificial
  Oligonucleotide nucleobase sequence motif
ttctatctacgcattg

SEQ ID NO: 87 - DNA, 16 bp, artificial
  Oligonucleotide nucleobase sequence motif
cttctatctacgcatt

SEQ ID NO: 88 - DNA, 17 bp, artificial
  Oligonucleotide nucleobase sequence motif
tcttctatctacgcatt

SEQ ID NO: 89 - DNA, 18 bp, artificial
  Oligonucleotide nucleobase sequence motif
ttcttctatctacgcatt

SEQ ID NO: 90 - DNA, 16 bp, artificial
  Oligonucleotide nucleobase sequence motif
tcttctatctacgcat

SEQ ID NO: 91 - DNA, 17 bp, artificial
  Oligonucleotide nucleobase sequence motif
ttcttctatctacgcat

SEQ ID NO: 92 - DNA, 18 bp, artificial
  Oligonucleotide nucleobase sequence motif
cttcttctatctacgcat

SEQ ID NO: 93 - DNA, 16 bp, artificial
  Oligonucleotide nucleobase sequence motif
ttcttctatctacgca

SEQ ID NO: 94 - DNA, 17 bp, artificial
  Oligonucleotide nucleobase sequence motif
cttcttctatctacgca

SEQ ID NO: 95 - DNA, 18 bp, artificial
  Oligonucleotide nucleobase sequence motif
gcttcttctatctacgca

SEQ ID NO: 96 - DNA, 16 bp, artificial
  Oligonucleotide nucleobase sequence motif
cttcttctatctacgc

SEQ ID NO: 97 - DNA, 16 bp, artificial
  Oligonucleotide nucleobase sequence motif
gcttcttctatctacg

SEQ ID NO: 98 - DNA, 16 bp, artificial
  Oligonucleotide nucleobase sequence motif
cgtggggcttcttcta

SEQ ID NO: 99 - DNA, 20 bp, artificial
  Oligonucleotide nucleobase sequence motif
tgacttggagaaaagcacaa

SEQ ID NO: 100 - DNA, 19 bp, artificial
  Oligonucleotide nucleobase sequence motif
ctgacttggagaaaagcac

SEQ ID NO: 101 - DNA, 16 bp, artificial
  Oligonucleotide nucleobase sequence motif
agagtcatcgtgctcc

SEQ ID NO: 102 - DNA, 20 bp, artificial
  Oligonucleotide nucleobase sequence motif
aagtactttaatagctcaaa

SEQ ID NO: 103 - DNA, 19 bp, artificial
  Oligonucleotide nucleobase sequence motif
aagtactttaatagctcaa

SEQ ID NO: 104 - DNA, 20 bp, artificial
  Oligonucleotide nucleobase sequence motif
gaagtactttaatagctcaa

SEQ ID NO: 105 - DNA, 16 bp, artificial
  Oligonucleotide nucleobase sequence motif
tactttaatagctcaa

SEQ ID NO: 106 - DNA, 18 bp, artificial
  Oligonucleotide nucleobase sequence motif
aagtactttaatagctca

SEQ ID NO: 107 - DNA, 19 bp, artificial
  Oligonucleotide nucleobase sequence motif
gaagtactttaatagctca

SEQ ID NO: 108 - DNA, 19 bp, artificial
  Oligonucleotide nucleobase sequence motif
agaagtactttaatagctc

SEQ ID NO: 109 - DNA, 20 bp, artificial
  Oligonucleotide nucleobase sequence motif
aagaagtactttaatagctc

SEQ ID NO: 110 - DNA, 17 bp, artificial
  Oligonucleotide nucleobase sequence motif
gaagtactttaatagct

SEQ ID NO: 111 - DNA, 20 bp, artificial
  Oligonucleotide nucleobase sequence motif
taagaagtactttaatagct

SEQ ID NO: 112 - DNA, 16 bp, artificial
  Oligonucleotide nucleobase sequence motif
gcaatgtgtaagaagt

SEQ ID NO: 113 - DNA, 275 bp, Homo sapiens
gacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttac

SEQ ID NO: 114 - DNA, 282 bp, Homo sapiens
gacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgc

SEQ ID NO: 115 - DNA, 266 bp, Homo sapiens
gacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagt

SEQ ID NO: 116 - DNA, 235 bp, Homo sapiens
caactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgc

SEQ ID NO: 117 - DNA, 229 bp, Homo sapiens
caactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagttacttcttac

SEQ ID NO: 118 - DNA, 20 bp, Homo Sapies
cttcttctatctacgcattg

SEQ ID NO: 119 - DNA, 275 bp, Homo Sapies
gtaagaagtactttaatagctcaaactcagagtcatcgtgctcccaattccaaagagattcctaaaagaggcaacttcggccgtttgagaagccagcgctcacccacccggggtctctgtgcattgacctttgggtgctgacttggagaaaagcacaaacacgaccagtcccatcctggctcccgtggggcttcttctatctacgcattgtatcgactgcattagttggactaagatgatgactcagttaaaggaggagacaaatgctgactgtc

SEQ ID NO: 120 - DNA, 282 bp, Homo Sapies
gcaatgtgtaagaagtactttaatagctcaaactcagagtcatcgtgctcccaattccaaagagattcctaaaagaggcaacttcggccgtttgagaagccagcgctcacccacccggggtctctgtgcattgacctttgggtgctgacttggagaaaagcacaaacacgaccagtcccatcctggctcccgtggggcttcttctatctacgcattgtatcgactgcattagttggactaagatgatgactcagttaaaggaggagacaaatgctgactgtc

SEQ ID NO: 121 - DNA, 266 bp, Homo Sapies
actttaatagctcaaactcagagtcatcgtgctcccaattccaaagagattcctaaaagaggcaacttcggccgtttgagaagccagcgctcacccacccggggtctctgtgcattgacctttgggtgctgacttggagaaaagcacaaacacgaccagtcccatcctggctcccgtggggcttcttctatctacgcattgtatcgactgcattagttggactaagatgatgactcagttaaaggaggagacaaatgctgactgtc

SEQ ID NO: 122 - DNA, 235 bp, Homo Sapies
gcaatgtgtaagaagtactttaatagctcaaactcagagtcatcgtgctcccaattccaaagagattcctaaaagaggcaacttcggccgtttgagaagccagcgctcacccacccggggtctctgtgcattgacctttgggtgctgacttggagaaaagcacaaacacgaccagtcccatcctggctcccgtggggcttcttctatctacgcattgtatcgactgcattagttg

SEQ ID NO: 123 - DNA, 229 bp, Homo Sapies
gtaagaagtaactttaatagctcaaactcagagtcatcgtgctcccaattccaaagagattcctaaaagaggcaacttcggccgtttgagaagccagcgctcacccacccggggtctctgtgcattgacctttgggtgctgacttggagaaaagcacaaacacgaccagtcccatcctggctcccgtggggcttcttctatctacgcattgtatcgactgcattagttg

SEQ ID NO: 124 - DNA, 20 bp, Homo Sapiens
atttgtctcctcctttaact

SEQ ID NO: 125 - DNA, 20 bp, Homo Sapiens
ttgtctcctcctttaactga

SEQ ID NO: 126 - DNA, 20 bp, Homo Sapiens
tgtctcctcctttaactgag

SEQ ID NO: 127 - DNA, 19 bp, Homo Sapiens
gtctcctcctttaactgag

SEQ ID NO: 128 - DNA, 20 bp, Homo Sapiens
gtctcctcctttaactgagt

SEQ ID NO: 129 - DNA, 19 bp, Homo Sapiens
tctcctcctttaactgagt

SEQ ID NO: 130 - DNA, 18 bp, Homo Sapiens
ctcctcctttaactgagt

SEQ ID NO: 131 - DNA, 20 bp, Homo Sapiens
cctcctttaactgagtcatc

SEQ ID NO: 132 - DNA, 20 bp, Homo Sapiens
tcctttaactgagtcatcat

SEQ ID NO: 133 - DNA, 18 bp, Homo Sapiens
cctttaactgagtcatca

SEQ ID NO: 134 - DNA, 20 bp, Homo Sapiens
cctttaactgagtcatcatc

SEQ ID NO: 135 - DNA, 19 bp, Homo Sapiens
ctttaactgagtcatcatc

SEQ ID NO: 136 - DNA, 19 bp, Homo Sapiens
ccaactaatgcagtcgata

SEQ ID NO: 137 - DNA, 20 bp, Homo Sapiens
ccaactaatgcagtcgatac

SEQ ID NO: 138 - DNA, 16 bp, Homo Sapiens
caactaatgcagtcga

SEQ ID NO: 139 - DNA, 16 bp, Homo Sapiens
caactaatgcagtcga

SEQ ID NO: 140 - DNA, 16 bp, Homo Sapiens
caactaatgcagtcga

SEQ ID NO: 141 - DNA, 18 bp, Homo Sapiens
caactaatgcagtcgata

SEQ ID NO: 142 - DNA, 19 bp, Homo Sapiens
caactaatgcagtcgatac

SEQ ID NO: 143 - DNA, 20 bp, Homo Sapiens
caactaatgcagtcgataca

SEQ ID NO: 144 - DNA, 16 bp, Homo Sapiens
aactaatgcagtcgat

SEQ ID NO: 145 - DNA, 16 bp, Homo Sapiens
aactaatgcagtcgat

SEQ ID NO: 146 - DNA, 16 bp, Homo Sapiens
aactaatgcagtcgat

SEQ ID NO: 147 - DNA, 17 bp, Homo Sapiens
aactaatgcagtcgata

SEQ ID NO: 148 - DNA, 18 bp, Homo Sapiens
aactaatgcagtcgatac

SEQ ID NO: 149 - DNA, 19 bp, Homo Sapiens
aactaatgcagtcgataca

SEQ ID NO: 150 - DNA, 20 bp, Homo Sapiens
aactaatgcagtcgatacaa

SEQ ID NO: 151 - DNA, 16 bp, Homo Sapiens
actaatgcagtcgata

SEQ ID NO: 152 - DNA, 16 bp, Homo Sapiens
actaatgcagtcgata

SEQ ID NO: 153 - DNA, 17 bp, Homo Sapiens
actaatgcagtcgatac

SEQ ID NO: 154 - DNA, 18 bp, Homo Sapiens
actaatgcagtcgataca

SEQ ID NO: 155 - DNA, 16 bp, Homo Sapiens
ctaatgcagtcgatac

SEQ ID NO: 156 - DNA, 16 bp, Homo Sapiens
ctaatgcagtcgatac

SEQ ID NO: 157 - DNA, 16 bp, Homo Sapiens
ctaatgcagtcgatac

SEQ ID NO: 158 - DNA, 17 bp, Homo Sapiens
ctaatgcagtcgataca

SEQ ID NO: 159 - DNA, 18 bp, Homo Sapies
ctaatgcagtcgatacaa

SEQ ID NO: 160 - DNA, 19 bp, Homo Sapiens
ctaatgcagtcgatacaat

SEQ ID NO: 161 - DNA, 16 bp, Homo sapiens
taatgcagtcgataca

SEQ ID NO: 162 - DNA, 16 bp, Homo sapiens
taatgcagtcgataca

SEQ ID NO: 163 - DNA, 18 bp, Homo sapiens
taatgcagtcgatacaat

SEQ ID NO: 164 - DNA, 16 bp, Homo sapiens
aatgcagtcgatacaa

SEQ ID NO: 165 - DNA, 16 bp, Homo sapiens
aatgcagtcgatacaa

SEQ ID NO: 166 - DNA, 16 bp, Homo sapiens
atgcagtcgatacaat

SEQ ID NO: 167 - DNA, 16 bp, Homo sapiens
atgcagtcgatacaat

SEQ ID NO: 168 - DNA, 16 bp, Homo sapiens
atgcagtcgatacaat

SEQ ID NO: 169 - DNA, 16 bp, Homo sapiens
agtcgatacaatgcgt

SEQ ID NO: 170 - DNA, 16 bp, Homo sapiens
agtcgatacaatgcgt

SEQ ID NO: 171 - DNA, 16 bp, Homo sapiens
gtcgatacaatgcgta

SEQ ID NO: 172 - DNA, 16 bp, Homo sapiens
gtcgatacaatgcgta

SEQ ID NO: 173 - DNA, 17 bp, Homo sapiens
gtcgatacaatgcgtag

SEQ ID NO: 174 - DNA, 18 bp, Homo sapiens
gtcgatacaatgcgtaga

SEQ ID NO: 175 - DNA, 19 bp, Homo sapiens
gtcgatacaatgcgtagat

SEQ ID NO: 176 - DNA, 20 bp, Homo sapiens
gtcgatacaatgcgtagata

SEQ ID NO: 177 - DNA, 16 bp, Homo sapiens
tcgatacaatgcgtag

SEQ ID NO: 178 - DNA, 16 bp, Homo sapiens
tcgatacaatgcgtag

SEQ ID NO: 179 - DNA, 19 bp, Homo sapiens
tcgatacaatgcgtagata

SEQ ID NO: 180 - DNA, 16 bp, Homo sapiens
cgatacaatgcgtaga

SEQ ID NO: 181 - DNA, 16 bp, Homo sapiens
cgatacaatgcgtaga

SEQ ID NO: 182 - DNA, 16 bp, Homo sapiens
cgatacaatgcgtaga

SEQ ID NO: 183 - DNA, 17 bp, Homo sapiens
cgatacaatgcgtagat

SEQ ID NO: 184 - DNA, 18 bp, Homo sapiens
cgatacaatgcgtagata

SEQ ID NO: 185 - DNA, 20 bp, Homo sapiens
cgatacaatgcgtagataga

SEQ ID NO: 186 - DNA, 16 bp, Homo sapiens
gatacaatgcgtagat

SEQ ID NO: 187 - DNA, 16 bp, Homo sapiens
gatacaatgcgtagat

SEQ ID NO: 188 - DNA, 17 bp, Homo sapiens
gatacaatgcgtagata

SEQ ID NO: 189 - DNA, 18 bp, Homo sapiens
gatacaatgcgtagatag

SEQ ID NO: 190 - DNA, 19 bp, Homo sapiens
gatacaatgcgtagataga

SEQ ID NO: 191 - DNA, 20 bp, Homo sapiens
gatacaatgcgtagatagaa

SEQ ID NO: 192 - DNA, 16 bp, Homo sapiens
atacaatgcgtagata

SEQ ID NO: 193 - DNA, 16 bp, Homo sapiens
atacaatgcgtagata

SEQ ID NO: 194 - DNA, 17 bp, Homo sapiens
atacaatgcgtagatag

SEQ ID NO: 195 - DNA, 18 bp, Homo sapiens
atacaatgcgtagataga

SEQ ID NO: 196 - DNA, 19 bp, Homo sapiens
atacaatgcgtagatagaa

SEQ ID NO: 197 - DNA, 16 bp, Homo sapiens
tacaatgcgtagatag

SEQ ID NO: 198 - DNA, 16 bp, Homo sapiens
tacaatgcgtagatag

SEQ ID NO: 199 - DNA, 17 bp, Homo sapiens
tacaatgcgtagataga

SEQ ID NO: 200 - DNA, 18 bp, Homo sapiens
tacaatgcgtagatagaa

SEQ ID NO: 201 - DNA, 17 bp, Homo sapiens
acaatgcgtagatagaa

SEQ ID NO: 202 - DNA, 19 bp, Homo sapiens
acaatgcgtagatagaaga

SEQ ID NO: 203 - DNA, 20 bp, Homo sapiens
acaatgcgtagatagaagaa

SEQ ID NO: 204 - DNA, 19 bp, Homo sapiens
caatgcgtagatagaagaa

SEQ ID NO: 205 - DNA, 16 bp, Homo sapiens
caatgcgtagatagaa

SEQ ID NO: 206 - DNA, 16 bp, Homo sapiens
aatgcgtagatagaag

SEQ ID NO: 207 - DNA, 17 bp, Homo sapiens
aatgcgtagatagaaga

SEQ ID NO: 208 - DNA, 18 bp, Homo sapiens
aatgcgtagatagaagaa

SEQ ID NO: 209 - DNA, 16 bp, Homo sapiens
atgcgtagatagaaga

SEQ ID NO: 210 - DNA, 17 bp, Homo sapiens
atgcgtagatagaagaa

SEQ ID NO: 211 - DNA, 18 bp, Homo sapiens
atgcgtagatagaagaag

SEQ ID NO: 212 - DNA, 16 bp, Homo sapiens
tgcgtagatagaagaa

SEQ ID NO: 213 - DNA, 17 bp, Homo sapiens
tgcgtagatagaagaag

SEQ ID NO: 214 - DNA, 18 bp, Homo sapiens
tgcgtagatagaagaagc

SEQ ID NO: 215 - DNA, 16 bp, Homo sapiens
gcgtagatagaagaag

SEQ ID NO: 216 - DNA, 16 bp, Homo sapiens
cgtagatagaagaagc

SEQ ID NO: 217 - DNA, 16 bp, Homo sapiens
tagaagaagccccacg

SEQ ID NO: 218 - DNA, 20 bp, Homo sapiens
ttgtgcttttctccaagtca

SEQ ID NO: 219 - DNA, 19 bp, Homo sapiens
gtgcttttctccaagtcag

SEQ ID NO: 220 - DNA, 16 bp, Homo sapiens
ggagcacgatgactct

SEQ ID NO: 221 - DNA, 20 bp, Homo sapiens
tttgagctattaaagtactt

SEQ ID NO: 222 - DNA, 19 bp, Homo sapiens
ttgagctattaaagtactt

SEQ ID NO: 223 - DNA, 20 bp, Homo sapiens
ttgagctattaaagtacttc

SEQ ID NO: 224 - DNA, 16 bp, Homo sapiens
ttgagctattaaagta

SEQ ID NO: 225 - DNA, 18 bp, Homo sapiens
tgagctattaaagtactt

SEQ ID NO: 226 - DNA, 19 bp, Homo sapiens
tgagctattaaagtacttc

SEQ ID NO: 227 - DNA, 19 bp, Homo sapiens
gagctattaaagtacttct

SEQ ID NO: 228 - DNA, 20 bp, Homo sapiens
gagctattaaagtacttctt

SEQ ID NO: 229 - DNA, 17 bp, Homo sapiens
agctattaaagtacttc

SEQ ID NO: 230 - DNA, 20 bp, Homo sapiens
agctattaaagtacttctta

SEQ ID NO: 231 - DNA, 20 bp, Homo sapiens
caatgcgtagatagaagaag